(19) United States
(12) Patent Application Publication
(10) Pub. No.: US 2003/0054417 A1
(43) Pub. Date: Mar. 20, 2003

(54) Epithelial protein lost in neoplasm (EPLIN)

(76) Inventors: David D. Chang, Calabasas, CA (US); Raymond S. Maul, Los Angeles, CA (US)

Correspondence Address: MICHAEL P. REED, PH.D., FISH & RICHARDSON P.C., Suite 500, 4350 La Jolla Village Drive, San Diego, CA, 92122, US

(21) Appl. No.: 09/783,732

(22) Filed: Feb. 13, 2001

Related U.S. Application Data
(63) Continuation-in-part of application No. 09/658,400, filed on Sep. 8, 2000.
(60) Provisional application No. 60/153,024, filed on Sep. 8, 1999.

(51) Int. Cl. G01N 33/574; C07H 21/04; C12N 9/64; C12P 21/02; C12N 5/06

(52) U.S. Cl. 435/7.23; 435/226; 435/69.1; 435/325; 435/320.1; 530/351; 536/23.2

(57) ABSTRACT

Nucleic acid molecule and polypeptide sequences encoding a novel tumor suppressor protein, EPLIN, are provided. Also included is a method for detecting a cell proliferative disorder associated with EPLIN. EPLIN is a marker that can be used diagnostically, prognostically and therapeutically over the course of cell proliferative disorders associated with EPLIN.

### CROSS REFERENCE TO RELATED APPLICATIONS

This application claims priority from U.S. patent application Ser. No. 09/658,400, filed Sep. 8, 2000, which claims priority from U.S. Provisional Application Ser. No. 60/153,024, filed Sep. 8, 1999, each disclosure of which is incorporated herein by reference.

### FIELD OF THE INVENTION

This invention relates generally to gene expression in normal and neoplastic cells, and specifically to a novel tumor suppressor gene, EPLIN (epithelial protein lost in neoplasm), and its gene products.

### BACKGROUND OF THE INVENTION

Progression of cancer in humans is associated with accumulation of genetic mutations. Most genes mutated in cancer are involved primarily in the maintenance of genomic integrity (Lengauer et al., Nature, 396:643, 1998) and the control of cell cycle progression (Sherr, Genes and Devel., 12:2984, 1998). These mutations in turn affect expression of a larger number of cellular genes which collectively are responsible for the changes in cell phenotype. Some of the differentially expressed genes function as oncogenes, while others behave as tumor suppressors to facilitate the development or progression of cancer (Weinberg, Annals of the New York Acad. of Sci., 758:331, 1995). As the number of genes that are differentially expressed in cancer far exceed the number of mutated genes, they provide an abundant source of targets that can be exploited to dissect the complex changes that underlie cellular transformation.

Cancer genes are broadly classified into “oncogenes” which, when activated, promote tumorigenesis, and “tumor suppressor genes” which, when nonfunctional, fail to suppress tumorigenesis. While these classifications provide a useful method for conceptualizing tumorigenesis, it is also possible that a particular gene may play differing roles depending upon the particular allelic form of that gene, its regulatory elements, the genetic background and the tissue environment in which it is operating.

Oncogenes are somatic cell genes that are mutated from their wild-type alleles (the art refers to these wild-type alleles as protooncogenes) into forms which are able to induce tumorigenesis under certain conditions. There is presently a substantial literature on known and putative oncogenes and the various alleles of these oncogenes.

Tumor suppressor genes are genes that, in their wild-type alleles, express proteins that suppress abnormal cellular proliferation. When the gene coding for a tumor suppressor protein is mutated, deleted or transcriptionally nonfunctional, the resulting absence of wild-type tumor suppressor protein expression promotes abnormal cellular proliferation. A number of well-studied human tumors and tumor cell lines have been shown to have missing or nonfunctional tumor suppressor genes. Examples of tumor suppression genes include, but are not limited to, the retinoblastoma susceptibility gene or RB gene, the p53 gene, the deleted in colon carcinoma (DCC) gene and the neurofibromatosis type 1 (NF-1) tumor suppressor gene (Weinberg, R. A. Science, 1991, 254:1138). Loss of function or inactivation of tumor suppressor genes may play a central role in the initiation and/or progression of a significant number of human cancers.

The present invention provides support for the idea that many cancers exhibit decreased EPLIN expression relative to their tissues of origin. The limitation and failings of the prior art to provide meaningful markers which correlate with the presence of cell proliferative disorders, such as cancer, has created a need for markers which can be used diagnostically, prognostically, and therapeutically over the course of such disorders. The present invention fulfills such a need.

### SUMMARY OF THE INVENTION

The present invention is based, at least in part, on the discovery of novel nucleic acid molecules and proteins encoded by such nucleic acid molecules, referred to herein as EPLIN proteins. The nucleic acid and protein molecules of the invention are useful as modulating agents in regulating a variety of cellular processes, e.g., cell-proliferative processes, or for detecting disorders, e.g., cell-proliferative disorders.

In one embodiment, a EPLIN nucleic acid molecule of the invention is at least 60%, 62%, 65%, 70%, 75%, 80%, 85%, 90%, 95%, 98% or more homologous to a nucleotide sequence (e.g., to the entire length of the nucleotide sequence) including SEQ ID NO:1, SEQ ID NO:3, SEQ NO:9, SEQ ID NO:11, SEQ ID NO:13 or a complement thereof. Preferably, a nucleic acid molecule of the invention is derived from a vertebrate source and more preferably a mammalian source.

In another embodiment, the isolated protein, preferably a EPLIN protein, has an amino acid sequence sufficiently homologous to the amino acid sequence of SEQ ID NO:2, SEQ ID NO:4, SEQ ID 10, SEQ ID NO:12, or SEQ ID NO:14. In a preferred embodiment, the protein, preferably a EPLIN protein, has an amino acid sequence at least about 41%, 42%, 45%, 50%, 55%, 59%, 60%, 65%, 70%, 75%, 80%, 81%, 85%, 90%, 95%, 98% or more homologous to an amino acid sequence including SEQ ID NO:2, SEQ ID NO:4, SEQ ID 10, SEQ ID NO:12, or SEQ ID NO:14 (e.g., the entire amino acid sequence of SEQ ID NO:2, SEQ ID NO:4, SEQ ID 10, SEQ ID NO:12, or SEQ ID NO:14). In another embodiment, the invention features fragments of the proteins having the amino acid sequence of SEQ ID NO:2, SEQ ID NO:4, SEQ ID 10, SEQ ID NO:12, or SEQ ID NO:14, wherein the fragment comprises at least 15 amino acids (e.g., contiguous amino acids) of the amino acid sequence of SEQ ID NO:2, SEQ ID NO:4, SEQ ID 10, SEQ ID NO:12, or SEQ ID NO:14, respectively. In another embodiment, the protein, preferably a EPLIN protein, has the amino acid sequence of SEQ ID NO:2, SEQ ID NO:4, SEQ ID 10, SEQ ID NO:12, or SEQ ID NO:14.

Another embodiment of the invention features an isolated protein, preferably a EPLIN protein, which is encoded by a nucleic acid molecule having a nucleotide sequence at least about 50%, 54%, 55%, 60%, 62%, 65%, 70%, 75%, 78%, 80%, 85%, 86%, 90%, 95%, 97%, 98% or more homologous to a nucleotide sequence (e.g., to the entire length of the nucleotide sequence) including SEQ ID NO:1, SEQ ID NO:3, SEQ ID NO:9, SEQ ID NO:11, SEQ ID NO:13, or a complement thereof. This invention further features an isolated protein, preferably a EPLIN protein, which is encoded by a nucleic acid molecule having a nucleotide sequence which hybridizes under stringent hybridization conditions to a nucleic acid molecule comprising the nucleotide sequence of SEQ ID NO:1, SEQ ID NO:3, SEQ ID NO:9, SEQ ID NO:11, SEQ ID NO:13, or a complement thereof.

In accordance with another aspect of the invention, an expression vector containing EPLIN nucleic acid is provided. The proteins of the present invention or biologically active portions thereof, can be operatively linked to a non-EPLIN polypeptide (e.g., heterologous amino acid sequences) to form fusion proteins. The invention further features antibodies, such as monoclonal or polyclonal antibodies, that specifically bind proteins of the invention, preferably EPLIN proteins. In addition, the EPLIN proteins or biologically active portions thereof can be incorporated into pharmaceutical compositions, which optionally include pharmaceutically acceptable carriers.

The invention further provides a method for identifying a compound that binds to EPLIN polypeptide that includes incubating components comprising the compound and EPLIN polypeptide under conditions sufficient to allow the components to interact and measuring the binding of the compound to EPLIN polypeptide.

The present invention provides a method for detecting the presence of a EPLIN polynucleotide, protein or polypeptide in a biological sample by contacting the biological sample with an agent capable of detecting a EPLIN polynucleotide, protein or polypeptide such that the presence of a EPLIN polynucleotide, protein or polypeptide is detected in the biological sample. Preferably, the invention provides a method of detecting a neoplastic cell in a sample by contacting a sample suspected of containing a neoplastic cell with a reagent that binds to an EPLIN-specific cell component and detecting binding of the reagent to the component.

In another aspect, the present invention provides a method for detecting the presence of EPLIN activity in a biological sample by contacting the biological sample with an agent capable of detecting an indicator of EPLIN activity such that the presence of EPLIN activity is detected in the biological sample. Preferably, the invention provides a method of detecting a cell proliferative disorder in a sample from a subject by contacting a first sample having, or suspected of having, a cell proliferative disorder with a reagent that binds to an EPLIN-specific cell component and detecting binding of the reagent to the component; contacting a second cell not having a cell proliferative disorder with a reagent that binds to an EPLIN-specific cell component and detecting binding of the reagent to the component; comparing the level of binding in the first sample with the level of binding in the second sample, wherein a decreased level of binding of the reagent to an EPLIN-specific cell component from the first sample is indicative of a cell proliferative disorder.

In another aspect, the invention provides a method for modulating EPLIN activity comprising contacting a cell capable of expressing EPLIN with an agent that modulates activity such that EPLIN activity in the cell is modulated. In one embodiment, the agent inhibits EPLIN activity. In another embodiment, the agent stimulates EPLIN activity. In one embodiment, the agent is an antibody that specifically binds to a EPLIN protein. In another embodiment, the agent modulates expression of EPLIN by modulating transcription of a EPLIN gene or translation of a EPLIN mRNA. In yet another embodiment, the agent is a nucleic acid molecule having a nucleotide sequence that is antisense to the coding strand of a EPLIN mRNA or a EPLIN gene.

The present invention also provides a diagnostic assay for identifying the presence or absence of a genetic alteration characterized by at least one of (i) aberrant modification or mutation of a gene encoding a EPLIN protein; (ii) mis-regulation of the EPLIN gene; and (iii) aberrant post-translational modification of a EPLIN protein, wherein a wild-type form of the gene encodes a protein with a EPLIN activity. Thus, the invention provides a kit useful for the detection of an EPLIN-specific cell component, the kit comprising carrier means containing one or more containers comprising a first container containing an EPLIN-specific binding reagent.

The invention further provides a method for treating a subject having a disorder characterized by aberrant EPLIN protein or nucleic acid expression or activity by administering an agent that is a EPLIN modulator to the subject. In one embodiment, the EPLIN modulator is a EPLIN protein. In another embodiment the EPLIN modulator is a EPLIN nucleic acid molecule. In yet another embodiment, the EPLIN modulator is a peptide, peptidomimetic, or other small molecule. Preferably, the disorder characterized by aberrant EPLIN protein or nucleic acid expression is a cellular growth related disorder, e.g., a cell proliferative disorder. Thus, the present invention provides a method of ameliorating a cell proliferative disorder associated with EPLIN, comprising administering to a subject with the disorder, a therapeutically effective amount of reagent that regulates EPLIN activity.

In a further aspect, the invention provides a method of gene therapy comprising introducing into cells of a host subject, an expression vector comprising a nucleotide sequence encoding EPLIN, in operable linkage with a promoter.

The invention further provides an isolated, synthetic, or recombinant polynucleotide comprising a epithelial lost in neoplasm (EPLIN) non-coding regulatory sequence.

In another embodiment, the invention provides a nucleic acid molecule comprising transcriptionl regulatory elements derived from EPLIN. In one aspect, the invention provides a nucleic acid construct the sequence of which comprises SEQ ID NO:15 operably associated to a heterologous sequence. In another aspect, the invention provides a nucleic acid construct the sequence of which comprises SEQ ID NO:16 operably associated to a heterologous sequence. In another aspect, the present invention provides vectors comprising the aforementioned nucleic acid constructs.

The invention further provides a method for screening for a compound that binds to a EPLIN non-coding regulatory sequence, comprising providing an isolated, synthetic, or recombinant polynucleotide comprising a EPLIN non-coding regulatory sequence and a test compound, contacting the polynucleotide with the test compound, and measuring the ability of the test compound to bind to the polynucleotide.

The invention further provides a method for screening for a compound that modulates EPLIN non-coding regulatory sequence activity, comprising providing a first polynucleotide comprising an isolated, synthetic, or recombinant EPLIN non-coding regulatory sequence operably linked to a heterogenous sequence, and a test compound, contacting the polynucleotide with the test compound, and measuring the ability of the test compound to modulate transcription of the heterogenous sequence.

The invention further provides a nucleic acid construct comprising at least one EPLIN non-coding regulatory sequence, and a heterologous nucleic acid sequence operatively linked to the regulatory sequence, wherein expression of the heterologous sequence is regulated by the non-coding sequence.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1A depicts a schematic diagram of human EPLIN-α and EPLIN-β cDNAs. The sequence of isoforms diverge at the 5′ end (indicated by the stripped and dotted boxes).

FIG. 1B depicts the deduced amino acid sequence of EPLIN-β.

FIG. 1C depicts the alignment of the EPLIN LIM domain sequence with the LIM domain of the mutant SREBP-2, KIAA0750, plant transcription factor SF3, and muscle LIM protein.

FIG. 2A depicts that EPLIN is preferentially expressed in epithelial cells as determined by Northern analysis.

FIG. 2B depicts the expression of EPLIN in different human primary cells by an immunoblot analysis.

FIG. 3A depicts, by Northern analysis, that the expression of EPLIN transcript is lost in epithelial cancer cells.

FIG. 3B depicts the expression of EPLIN proteins in different prostate cancer cell lines and xenograft tumors as determined by an immunoblot analysis.

FIG. 3C depicts the expression of EPLIN proteins in different breast cancer cell lines as determined by an immunoblot analysis.

FIG. 3D depicts the expression of EPLIN transcripts in different breast cancer cell lines as determined by a Northern analysis.

FIG. 4A depicts the relative amount of EPLIN isoforms in HOK18C (a HPV-immortalized human oral keratinocyte cell line) and BeWo (a human choriocarcinoma cell line) as determined by an immunoblot analysis.

FIG. 4B depicts the subcellular localization of EPLIN as determined by in situ immunofluorescence using anti-EPLIN antibodies and Texas Red-conjugated goat anti-rabbit IgG secondary antibody.

FIG. 4C depicts the staining of actin stress fibers with Oregon Green-phalloidin.

FIG. 4D depicts the subcellular localization of EPLIN as determined by in situ immunofluorescence using anti-EPLIN antibodies and fluorescein isothiocyanate-conjugated goat anti-rabbit IgG secondary antibody.

FIG. 4E depicts the staining of actin stress fibers with Texas Red-phalloidin.

FIG. 5A depicts U2-OS osteosarcoma cultured without expression of EPLIN-α.

FIG. 5B depicts U2-OS osteosarcoma cultured with expression of EPLIN-α.

FIG. 5C depicts U2-OS osteosarcoma cultured without expression of EPLIN-β.

FIG. 5D depicts U2-OS osteosarcoma cultured with expression of EPLIN-β.

FIG. 5E depicts the levels of EPLIN expression in the U2-OS cells cultures minus (no induction) and plus (induction) doxycycline as determined by an immunoblot analysis.

FIG. 5F depicts the growth of U2-OS cells presented as the ratio of cell numbers with and without EPLIN induction.

FIG. 6 depicts the amnino acid sequence alignment of mouse EPLIN-β (mEPLIN-β), mouse EPLIN-α (mEPLIN-α), human EPLIN-β (hEPLIN-β) and zebrafish EPLIN (zfEPLIN). Seven distinct conserved regions (I-VII) are indicated in the diagram.

FIG. 7 depicts a diagram of the BAC clone RCPI22-121N19 containing Exons 2 to 11 of the mouse EPLIN gene. Primers complementary to mouse EPLIN cDNA were used to sequence across the exon/intron boundaries.

FIG. 8 depicts the detection of mEPLIN-α and mEPLIN-β in various tissues. Panel A is an autoradiograph showing that mEPLIN-α is expressed in mouse embryos, adult lung and spleen tissue, while mEPLIN-β is expressed in the kidney, lung, liver, and testis tissue. Panel B is a bar graph depicting the results of the semi-quantitative RT-PCR assay shown in Panel A. Panel C shows an immunoblot analysis of mEPLIN-α and mEPLINβ protein expression in various tissues using anti-EPLIN antisera.

FIG. 9 depicts the subcellular localization of mEPLIN-α, mEPLIN-β and zfEPLIN using in situ immunofluorescence microscopy. Human Saos2 osteosarcoma cells were transiently transfected with the mEPLIN-α (Panels A and B), mEPLIN-β (FIG. 9, Panels C and D), and zfEPLIN (FIG. 9, Panels E and F) expression constructs. Twenty-four hours after the transfection, the cells were fixed and then stained for the transfected EPLIN (anti-Flag mAb) (Panels A, C, and E) and the stress fibers (Rhodamine-Phalloidin) (Panels B, D, and F).

FIG. 10 depicts the genomic organization of the human EPLIN. Panel A shows a diagrammatic representation of the human EPLIN gene. Restriction enzymes cleavage sites for BamH1 (B) and EcoR1 (R) are denoted. Panel B shows the sequence of exon-intron boundaries in the EPLIN gene. The conserved AG and GT dinucleotides and polypyrimidine tract at the 3′ splice sites are shown. The alternative 3′ splice site in intron 8 is indicated.

FIG. 11 depicts the analysis of the human EPLIN-α promoter region. Panel A shows the nucleotide sequences flanking the 5′ end of EPLIN-α transcripts. Panel B shows a series of constructs containing the 5′ flanking region of EPLIN-α that were tested for promoter activity. The results are average+standard deviation from three independent experiments.

FIG. 12 depicts the analysis of human EPLIN-β promoter region. Panel A shows the nucleotide sequences flanking the 5′ end of EPLIN-β transcripts. Panel B shows the 5′ flanking region of EPLIN-β that was tested for promoter activity. The results are average+standard deviation from three independent experiments.

FIG. 13 depicts data indicating that EPLIN-α is a primary response gene. Panel A shows quiescent NIH3T3 cells that were stimulated with 15% serum. Panel B shows the results of hybridization assays as quantified using a phosphorimager. Panel C shows the results of serum stimulation carried out in the presence of puromycin (10 μg/ml) to inhibit de novo protein synthesis. Panel D shows the result of co-transfecting pGL3/0.7 kb EPLIN-α with RhoA^{L63}, Cdc42^{L61}, or Rac1^{L61}.

### DETAILED DESCRIPTION

The present invention is based, at least in part, on the discovery of novel molecules, referred to herein as “EPLIN” (epithelial protein lost in neoplasm) or “EPLIN” nucleic acid and polypeptide molecules, which play a role in or function in signaling pathways associated with cellular growth. EPLIN is a novel tumor suppressor gene encoding novel cytoskeletal proteins preferentially expressed in epithelial cells. For example, the invention provides two EPLIN isoforms, a 600 amino acid EPLIN-α (SEQ ID NO:2) and a 759 amino acid EPLIN-β (SEQ ID NO:4), are detected in primary human epithelial cells of oral mucosa, prostate and mammary glands. Both EPLIN isoforms localize to filamentous actin and suppress cell proliferation when overexpressed. These findings indicate that the loss of EPLIN seen in cancer cells may play a role in cancer progression. In addition, the invention provides a mouse EPLIN-α polypeptide (SEQ ID NO:10), a mouse EPLIN-β polypeptide (SEQ ID NO:12) and an EPLIN polypeptide derived from a zebrafish (SEQ ID NO:14). The invention further provides a nucleic acid molecule (SEQ ID NO:1) encoding the amino acid sequence of human EPLIN-α and a nucleic acid molecule (SEQ ID NO:3) encoding the amino acid sequence of human EPLIN-β. The invention further provides nucleic acid molecules (SEQ ID NO:9 and SEQ ID NO:11) encoding the amino acid sequence of EPLIN mouse α and mouse β, respectively. Finally, the invention provides a nucleic acid molecule (SEQ ID NO:13) encoding the amino acid sequence of zebrafish EPLIN-α. Based on these discoveries, it is an object of the invention to provide compounds, and pharmaceutical compositions thereof, which modulate cell proliferation. It is a further object of the invention to provide methods of detecting a cell proliferative disorder by detecting EPLIN expression.

The EPLIN protein, fragments thereof, and derivatives and other variants of the sequence in SEQ ID NO:2, SEQ ID NO:4, SEQ ID NO:10, SEQ ID NO:12 or SEQ ID NO:14, are collectively referred to as “polypeptides or proteins of the invention” or “EPLIN polypeptides or proteins.” Nucleic acid molecules encoding such polypeptides or proteins are collectively referred to as “nucleic acids of the invention” or “EPLIN polynucleotide” or “EPLIN nucleic acids.” EPLIN molecules refer to EPLIN nucleic acids, polypeptides, and antibodies.

As used herein, the term “nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded, but preferably is double-stranded DNA.

The term “isolated or purified nucleic acid molecule” includes nucleic acid molecules that are separated from other nucleic acid molecules that are present in the natural source of the nucleic acid. For example, with respect to genomic DNA, the term “isolated” includes nucleic acid molecules that are separated from the chromosome with which the genomic DNA is naturally associated. Preferably, an “isolated” nucleic acid is free of sequences that naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences that naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, the term “hybridizes under stringent conditions” describes conditions for hybridization and washing. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. A preferred, non-limiting example of stringent hybridization conditions are hybridization in 6×sodium chloride/sodium citrate (SSC) at about 45° C., followed by one or more washes in 0.2×SSC, 0.1% SDS at 50-65° C.

As used herein, a “naturally occurring” nucleic acid molecule refers to an RNA or DNA molecule having a nucleotide sequence that occurs in nature (e.g., encodes a natural protein).

As used herein, the terms “gene” and “recombinant gene” refer to nucleic acid molecules that include an open reading frame encoding an EPLIN protein, preferably a mammalian EPLIN protein, and further can include non-coding regulatory sequences and introns.

An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of EPLIN protein having less than about 30%, 20%, 10% and more preferably 5% (by dry weight), of non-EPLIN protein (also referred to herein as a “contaminating protein”), or of chemical precursors or non-EPLIN chemicals. When the EPLIN protein or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

A “non-essential” amino acid residue is a residue that can be altered from the wild-type sequence of EPLIN (e.g., the sequence of SEQ ID NO:2, SEQ ID NO:4, SEQ ID NO:10, SEQ ID NO:12 or SEQ ID NO:14) without abolishing or more preferably, without substantially altering a biological activity of the EPLIN protein, whereas an “essential” amino acid residue results in such a change. For example, amino acid residues that are conserved among the polypeptides of the present invention are predicted to be particularly unamenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an EPLIN protein is preferably replaced with another amino acid residue from the same side chain family. Alternatively, in another embodiment, mutations can be introduced randomly along all or part of an EPLIN coding sequence, such as by saturation mutagenesis, and the resultant mutants can be screened for EPLIN biological activity to identify mutants that retain activity. Following mutagenesis of SEQ ID NO:1, 3, 9, 11 or 13, the encoded protein can be expressed recombinantly and the activity of the protein can be determined.

As used herein, a “biologically active portion” of an EPLIN protein includes a fragment of an EPLIN protein that participates in an interaction between an EPLIN molecule and a non-EPLIN molecule. Biologically active portions of an EPLIN protein include peptides comprising amino acid sequences sufficiently homologous to or derived from the amino acid sequence of the EPLIN protein, e.g., the amino acid sequence shown in SEQ ID NO:2, 4, 10, 12 or 14, which include less amino acids than the full length EPLIN protein and exhibit at least one activity of an EPLIN protein, such as tumor suppressor activity. Typically, biologically active portions comprise a domain or motif with at least one activity of the EPLIN protein. A biologically active portion of an EPLIN protein can be a polypeptide that is, for example, 10, 25, 50, 100, 200, 300 or more amino acids in length. Biologically active portions of an EPLIN protein can be used as targets for developing agents that modulate an EPLIN mediated activity.

Calculations of homology or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol, 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within the invention) is using a Blossum 62 scoring matrix with a gap open penalty of 12, a gap extend penalty of 4, and a frameshift gap penalty of 5.

The percent identity between two amino acid or nucleotide sequences can be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol., 215:403-10. BLAST nucleotide searches can be performed with the NBLAST program, score=100, wordlength=12 to obtain nucleotide sequences homologous to EPLIN nucleic acid molecules of the invention. BLAST protein searches can be performed with the XBLAST program, score=50, wordlength=3 to obtain amino acid sequences homologous to EPLIN protein molecules of the invention. To obtain gapped alignments for comparison purposes, Gapped BLAST can be utilized as described in Altschul et al., (1997) Nucleic Acids Res., 25(17):3389-3402. When utilizing BLAST and Gapped BLAST programs, the default parameters of the respective programs (e.g., XBLAST and NBLAST) can be used. See http://www.ncbi.nlm.nih.gov.

“Misexpression or aberrant expression,” as used herein, refers to a non-wild type pattern of gene expression at the RNA or protein level. It includes: expression at non-wild type levels, i.e., over or underexpression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

“Subject,” as used herein, can refer to a mammal, e.g., a human, or to an experimental animal or disease model. The subject also can be a non-human animal, e.g., a horse, cow, goat, or other domestic animal.

In one embodiment, an isolated nucleic acid molecule of the present invention comprises a nucleotide sequence which is at least about 50%, 54%, 55%, 60%, 62%, 65%, 70%, 75%, 78%, 80%, 85%, 86%, 90%, 95%, 97%, 98% or more homologous to the nucleotide sequence (e.g., to the entire length of the nucleotide sequence) shown in SEQ ID NO:1, SEQ ID NO:3, SEQ ID NO:9, SEQ ID NO:11, SEQ ID NO:13, or a portion of any of these nucleotide sequences.

Moreover, the nucleic acid molecule of the invention can comprise only a portion of the nucleic acid sequence of EPLIN SEQ ID NO:1, SEQ ID NO:3, SEQ ID NO:9, SEQ ID NO:11, SEQ ID NO:13, for example a fragment which can be used as aprobe or primer or a fragment encoding a biologically active portion of a protein. The nucleotide sequence determined from the cloning of the EPLIN human, mouse and zebrafish genes allows for the generation of probes and primers designed for use in identifying and/or cloning other EPLIN family members, as well as EPLIN homologues from additional species. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1, SEQ ID NO:3, SEQ ID NO:9, SEQ ID NO:11, SEQ ID NO:13, of an anti-sense sequence of SEQ ID NO:1, SEQ ID NO:3, SEQ ID NO:9, SEQ ID NO:11, SEQ ID NO:13, or of a naturally occurring allelic variant or mutant of SEQ ID NO:1, SEQ ID NO:3, SEQ ID NO:9, SEQ ID NO:11, SEQ ID NO:13. In an exemplary embodiment, a nucleic acid molecule of the present invention comprises a nucleotide sequence which is at least 350, 400, 450, 500, 550, 600, 650, 700, 750, or 800 nucleotides in length and hybridizes under stringent hybridization conditions to a nucleic acid molecule of SEQ ID NO:1, SEQ ID NO:3, SEQ ID NO:9, SEQ ID NO:11, SEQ ID NO:13.

Probes based on the EPLIN nucleotide sequences can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissues which misexpress a EPLIN protein, such as by measuring a level of a EPLIN-encoding nucleic acid in a sample of cells from a subject e.g., detecting EPLIN mRNA levels or determining whether a genomic EPLIN gene has been mutated or deleted.

A nucleic acid fragment encoding a “biologically active portion of a EPLIN protein” can be prepared by isolating a portion of the nucleotide sequence of SEQ ID NO:1, SEQ ID NO:3, SEQ ID NO:9, SEQ ID NO:11, SEQ ID NO:13, which encodes a polypeptide having a biological activity, expressing the encoded portion of the EPLIN protein (e.g., by recombinant expression in vitro) and assessing the activity of the encoded portion of the EPLIN protein.

In another embodiment, an isolated nucleic acid molecule of the invention has a nucleotide sequence encoding a protein having an amino acid sequence shown in SEQ ID NO:2, SEQ ID NO:4, SEQ ID NO:10, SEQ ID NO:12, or SEQ ID NO:14.

In addition to the EPLIN nucleotide sequences shown in SEQ ID NO:1, SEQ ID NO:3, SEQ ID NO:9, SEQ ID NO:11, SEQ ID NO:13, it will be appreciated by those skilled in the art that DNA sequence polymorphisms that lead to changes in the amino acid sequences of the proteins may exist within a population (e.g., the human population). Such genetic polymorphism in the EPLIN genes may exist among individuals within a population due to natural allelic variation. As used herein, the terms “gene” and “recombinant gene” refer to nucleic acid molecules which include an open reading frame encoding an EPLIN protein, preferably a mammalian EPLIN protein, and can further include non-coding regulatory sequences, and introns. Such natural allelic variations include both functional and non-functional EPLIN proteins and can typically result in 1-5% variance in the nucleotide sequence of a EPLIN gene. Any and all such nucleotide variations and resulting amino acid polymorphisms in EPLIN genes that are the result of natural allelic variation and that do not alter the functional activity of a EPLIN protein are intended to be within the scope of the invention.

Moreover, nucleic acid molecules encoding other EPLIN family members and, thus, which have a nucleotide sequence which differs from the EPLIN sequences of SEQ ID NO:1, SEQ ID NO:3, SEQ ID NO:9, SEQ ID NO:11, SEQ ID NO:13, are intended to be within the scope of the invention. For example, another EPLIN cDNA can be identified based on the nucleotide sequence of human, mouse and zebrafish EPLIN disclosed herein. Moreover, nucleic acid molecules encoding EPLIN proteins from different species, and thus which have a nucleotide sequence which differs from the EPLIN sequences of SEQ ID NO:1, SEQ ID NO:3, SEQ ID NO:9, SEQ ID NO:11, SEQ ID NO:13 are intended to be within the scope of the invention. For example, any vertebrate EPLIN cDNA can be identified based on the nucleotide sequence of a human, mouse or zebrafish EPLIN.

Nucleic acid molecules corresponding to natural allelic variants and homologues of the EPLIN cDNAs of the invention can be isolated based on their homology to the EPLIN nucleic acids disclosed herein using the cDNAs disclosed herein, or a portion thereof, as a hybridization probe according to standard hybridization techniques under stringent hybridization conditions.

Accordingly, in another embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:1, SEQ ID NO:3, SEQ ID NO:9, SEQ ID NO:11, SEQ ID NO:13. In other embodiment, the nucleic acid is at least 30, 50, 100, 150, 200, 250, 300, 350, 400, 450, 500, 550, or 600 nucleotides in length. As used herein, the term “hybridizes under stringent conditions” is intended to describe conditions for hybridization and washing under which nucleotide sequences at least 30%, 40%, 50%, or 60% homologous to each other typically remain hybridized to each other. Preferably, the conditions are such that sequences at least about 70%, more preferably at least about 80%, even more preferably at least about 85% or 90% homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6×sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to the sequence of SEQ ID NO:1, SEQ ID NO:3, SEQ ID NO:9, SEQ ID NO:11, SEQ ID NO:13 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

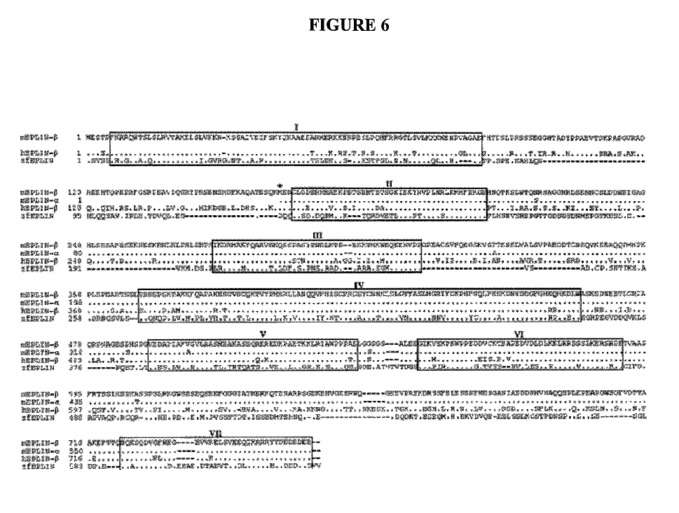

In addition to naturally-occurring allelic variants of the EPLIN sequences that may exist in the population, the skilled artisan will further appreciate that changes can be introduced by mutation into the nucleotide sequences of SEQ ID NO:1, SEQ ID NO:3, SEQ ID NO:9, SEQ ID NO:11, SEQ ID NO:13, thereby leading to changes in the amino acid sequence of the encoded proteins, without altering the functional ability of the proteins. For example, nucleotide substitutions leading to amino acid substitutions at “non-essential” amino acid residues can be made in the sequence of SEQ ID NO:1, SEQ ID NO:3, SEQ ID NO:9, SEQ ID NO:11, SEQ ID NO:13. A “non-essential” amino acid residue is a residue that can be altered from the wild-type sequence of (e.g., the sequence of SEQ ID NO:2, SEQ ID NO:4, SEQ ID NO:10, SEQ ID NO:12, or SEQ ID NO:14) without altering the biological activity, whereas an “essential” amino acid residue is required for biological activity. For example, amino acid residues that are conserved among the EPLIN proteins of the present invention (see e.g., FIG. 6, domains I-VII) are predicted to be particularly unamenable to alteration. Furthermore, additional amino acid residues that are conserved between the EPLIN proteins of the present invention and other EPLIN family members are not likely to be amenable to alteration.

The invention also provides an isolated nucleic acid molecule consisting essentially of a nucleic acid molecule encoding a polypeptide having the amino acid sequence of SEQ ID NO:2, 4, 10, 12 or 14. Nucleic acid molecules of the invention include DNA, cDNA and RNA sequences that encode EPLIN. It is understood that all nucleic acid molecules encoding all or a portion of EPLIN are also included herein, as long as they encode a polypeptide with EPLIN activity. Such nucleic acid molecules include naturally occurring, synthetic, and intentionally manipulated nucleic acid molecules. For example, EPLIN nucleic acid molecule may be subjected to site-directed mutagenesis. The nucleic acid molecule sequence for EPLIN also includes antisense sequences. The nucleic acid molecules of the invention include sequences that are degenerate as a result of the genetic code. There are 20 natural amino acids, most of which are specified by more than one codon. Therefore, all degenerate nucleotide sequences are included in the invention as long as the amino acid sequence of EPLIN polypeptide encoded by the nucleotide sequence is functionally unchanged. In addition, the invention also includes a nucleic acid molecule consisting essentially of a polynucleotide sequence encoding a polypeptide having an amino acid sequence of SEQ ID NO:2, 4, 10, 12 or 14 and having at least one epitope for an antibody immunoreactive with EPLIN polypeptide.

Nucleic acids of the invention can be chosen for having codons, which are preferred or non-preferred, for a particular expression system. E.g., the nucleic acid can be one in which at least one codon, at preferably at least 10%, or 20% of the codons has been altered such that the sequence is optimized for expression in E. coli, yeast, human, insect, or chinese hamster ovary (CHO) cells.

Nucleic acid variants can be naturally occurring, such as allelic variants (same locus), homologs (different locus), and orthologs (different organism) or can be non-naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared with the encoded product).

Orthologs, homologs, and allelic variants can be identified using methods known in the art. These variants comprise a nucleotide sequence encoding a polypeptide that is 50%, at least about 55%, typically at least about 70-75%, more typically at least about 80-85%, and most typically at least about 90-95% or more identical to the amino acid sequence shown in SEQ ID NO:2, 4, 10, 12 or 14, or a fragment of these sequences. Such nucleic acid molecules can readily be identified as being able to hybridize under stringent conditions, to the nucleotide sequence shown in SEQ ID NO:1, 3, 9, 11 or 13, or a fragment of these sequences. Nucleic acid molecules corresponding to orthologs, homologs, and allelic variants of the EPLIN cDNAs of the invention can further be isolated by mapping to the same chromosome or locus as the EPLIN gene. Preferred variants include those that are correlated with a tumor suppressor activity.

Allelic variants of EPLIN include both functional and non-functional proteins. Functional allelic variants are naturally occurring amino acid sequence variants of the EPLIN protein within a population that maintain the ability to function as a tumor suppressor protein. Functional allelic variants typically will contain only conservative substitution of one or more amino acids of SEQ ID NO:2, 4, 10, 12 or 14, or substitution, deletion or insertion of non-critical residues in non-critical regions of the protein. Non-functional allelic variants are naturally occurring amino acid sequence variants of EPLIN. Non-functional allelic variants will typically contain a non-conservative substitution, a deletion, or insertion, or premature truncation of the amino acid sequence of SEQ ID NO:2, 4, 10, 12 or 14, or a substitution, insertion, or deletion in critical residues or critical regions of the protein.

Nucleic acid molecules encoding EPLIN include the nucleotide sequence of SEQ ID NO:1, 3, 9, 11 or 13, as well as nucleic acid sequences complementary to those sequences. A complementary sequence may include an antisense nucleotide. When the sequence is RNA, the deoxynucleotides A, G, C, and T of a sequence of the invention are replaced by ribonucleotides A, G, C, and U, respectively. Also included in the invention are fragments of the above-described nucleic acid sequences that are at least 15 bases in length, which is sufficient to permit the fragment to selectively hybridize to DNA that encodes a polypeptide of the invention under physiological conditions.

In another aspect, the invention features, an isolated nucleic acid molecule that is antisense to EPLIN. An “antisense” nucleic acid can include a nucleotide sequence that is complementary to a “sense” nucleic acid encoding a protein, e.g., complementary to the coding strand of a double-stranded cDNA molecule or complementary to an mRNA sequence. The antisense nucleic acid can be complementary to an entire EPLIN coding strand, or to only a portion thereof. In another embodiment, the antisense nucleic acid molecule is antisense to a “noncoding region” of the coding strand of a nucleotide sequence encoding EPLIN (e.g., the 5′ and 3′ untranslated regions).

An antisense nucleic acid can be designed such that it is complementary to the entire coding region of EPLIN mRNA, but more preferably is an oligonucleotide that is antisense to only a portion of the coding or noncoding region of EPLIN mRNA. For example, the antisense oligonucleotide can be complementary to the region surrounding the translation start site of EPLIN mRNA, e.g., between the −10 and +10 regions of the target gene nucleotide sequence. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions with procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

Given the coding strand sequences encoding EPLIN disclosed herein, antisense nucleic acids of the invention can be designed according to the rules of Watson and Crick base pairing. The antisense nucleic acid molecule can be complementary to the entire coding region of EPLIN mRNA, but more preferably is an oligonucleotide that is antisense to only a portion of the coding or noncoding region of EPLIN mRNA. For example, the antisense oligonucleotide can be complementary to the region surrounding the translation start site of EPLIN mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., by direct injection at a tissue site), or generated in situ such that they hybridize with or bind to cellular mRNA and/or genomic DNA encoding an EPLIN protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong polymerase II or polymerase III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual b-units, the strands run parallel to each other (Gaultier et al. (1987) Nucleic Acids. Res., 15:6625-6641). The antisense nucleic acid molecule can also comprise a 2′-o-methylribonucleotide (Inoue et al. (1987) Nucleic Acids Res., 15:6131-6148) or a chimeric RNA-DNA analogue (Inoue et al. (1987) FEBS Lett., 215:327-330).

In still another embodiment, an antisense nucleic acid of the invention is a ribozyme. A ribozyme having specificity for an EPLIN-encoding nucleic acid can include one or more sequences complementary to the nucleotide sequence of an EPLIN cDNA disclosed herein, and a sequence having known catalytic sequence responsible for mRNA cleavage (see U.S. Pat. No. 5,093,246 or Haselhoff and Gerlach (1988) Nature, 334:585-591). For example, a derivative of a Tetrahymena L-19 IVS RNA can be constructed in which the nucleotide sequence of the active site is complementary to the nucleotide sequence to be cleaved in an EPLIN-encoding mRNA. See, e.g., Cech et al. U.S. Pat. No. 4,987,071; and Cech et al. U.S. Pat. No. 5,116,742. Alternatively, EPLIN mRNA can be used to select a catalytic RNA having a specific ribonuclease activity from a pool of RNA molecules. See, e.g., Bartel, D. and Szostak, J. W. (1993) Science, 261:1411-1418.

EPLIN gene expression can be inhibited by targeting nucleotide sequences complementary to the regulatory region of the EPLIN (e.g., the EPLIN promoter and/or enhancers discussed below) to form triple helical structures that prevent transcription of the EPLIN gene in target cells. See, generally, Helene, C. (1991) Anticancer Drug Des., 6(6):569-84; Helene, C. et al. (1992) Ann. N.Y. Acad. Sci., 660:27-36; and Maher, L. J. (1992) Bioassays, 14(12):807-15. The potential sequences that can be targeted for triple helix formation can be increased by creating a “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

A nucleic acid molecule of the invention can be obtained by several methods. For example, the nucleic acid molecule can be isolated using hybridization techniques that are well known in the art. These include, but are not limited to: (1) hybridization of genomic or cDNA libraries with probes to detect homologous nucleotide sequences and (2) antibody screening of expression libraries to detect cloned DNA fragments with shared structural features.

Preferably a EPLIN nucleic acid molecule of the invention is derived from a vertebrate organism, more preferably a mammalian organism, and most preferably from a primate organism. Screening procedures that rely on nucleic acid hybridization make it possible to isolate any gene sequence from any organism, provided the appropriate probe is available. Oligonucleotide probes, which correspond to a part of the sequence encoding the protein in question, can be synthesized chemically. This requires that short, oligopeptide stretches of amino acid sequence must be known. The DNA sequence encoding the protein can be deduced from the genetic code, however, the degeneracy of the code must be taken into account. It is possible to perform a mixed addition reaction when the sequence is degenerate. This includes a heterogeneous mixture of denatured double-stranded DNA. For such screening, hybridization is preferably performed on either single-stranded DNA or denatured double-stranded DNA. Hybridization is particularly useful in the detection of cDNA clones derived from sources where an extremely low amount of mRNA sequences relating to the polypeptide of interest are present. In other words, by using stringent hybridization conditions directed to avoid non-specific binding, it is possible, for example, to allow the autoradiographic visualization of a specific cDNA clone by the hybridization of the target DNA to that single probe in the mixture that is its complete complement (Wallace et al., Nucl. Acid Res., 9:879, 1981).

The development of specific DNA sequences encoding EPLIN can also be obtained by: (1) isolation of double-stranded DNA sequences from the genomic DNA; (2) chemical manufacture of a DNA sequence to provide the necessary codons for the polypeptide of interest; and (3) in vitro synthesis of a double-stranded DNA sequence by reverse transcription of mRNA isolated from a eukaryotic donor cell. In the latter case, a double-stranded DNA complement of mRNA is eventually formed which is generally referred to as cDNA.

Of the three above-noted methods for developing specific DNA sequences for use in recombinant procedures, the isolation of genomic DNA isolates is the least common. This is especially true when it is desirable to obtain the microbial expression of mammalian polypeptides due to the presence of introns.

The synthesis of DNA sequences is frequently the method of choice when the entire sequence of amino acid residues of the desired polypeptide product is known. When the entire sequence of amino acid residues of the desired polypeptide is not known, the direct synthesis of DNA sequences is not possible and the method of choice is the synthesis of cDNA sequences. Among the standard procedures for isolating cDNA sequences of interest is the formation of plasmid- or phage-carrying cDNA libraries that are derived from reverse transcription of mRNA that is abundant in donor cells that have a high level of genetic expression. When used in combination with polymerase chain reaction technology, even rare expression products can be cloned. In those cases where significant portions of the amino acid sequence of the polypeptide are known, the production of labeled single or double-stranded DNA or RNA probe sequences duplicating a sequence putatively present in the target cDNA may be employed in DNA/DNA hybridization procedures which are carried out on cloned copies of the cDNA which have been denatured into a single-stranded form (Jay et al., Nucl. Acid Res., 11:2325, 1983).

A cDNA expression library, such as lambda gt11, can be screened indirectly for EPLIN peptides having at least one epitope, using antibodies specific for EPLIN. Such antibodies can be either polyclonally or monoclonally-derived and used to detect expression product indicative of the presence of EPLIN cDNA.

DNA sequences encoding EPLIN can be expressed in vitro by DNA transfer into a suitable host cell. “Host cells” are cells in which a vector can be propagated and its DNA expressed. The term also includes any progeny of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

In the present invention, a EPLIN polynucleotide sequences may be inserted into a recombinant expression vector. The term “recombinant expression vector” refers to a plasmid, virus or other vehicle known in the art that has been manipulated by insertion or incorporation of the EPLIN genetic sequences. Such expression vectors contain a promoter sequence that facilitates the efficient transcription of the inserted genetic sequence of the host. The expression vector typically contains an origin of replication, a promoter, as well as specific genes that allow phenotypic selection of the transformed cells. Vectors suitable for use in the present invention include, but are not limited to the T7-based expression vector for expression in bacteria (Rosenberg et al., Gene, 56:125, 1987), the pMSXND expression vector for expression in mammalian cells (Lee and Nathans, J. Biol. Chem., 263:3521, 1988) and baculovirus-derived vectors for expression in insect cells. The DNA segment can be present in the vector operably linked to regulatory elements, for example, a promoter (e.g., T7, metallothionein I, or polyhedrin promoters).

Polynucleotide sequences encoding EPLIN can be expressed in either prokaryotes or eukaryotes. Hosts can include microbial, yeast, insect, and mammalian organisms. Methods of expressing DNA sequences having eukaryotic or viral sequences in prokaryotes are well known in the art. Biologically functional viral and plasmid DNA vectors capable of expression and replication in a host are known in the art. Such vectors are used to incorporate DNA sequences of the invention.

The invention further provides substantially pure EPLIN polypeptides consisting essentially of the amino acid sequence of SEQ ID NO:2, 4, 10, 12 or 14. The term “substantially pure” as used herein refers to EPLIN polypeptide that is substantially free of other proteins, lipids, carbohydrates or other materials with which it is naturally associated. One skilled in the art can purify EPLIN using standard techniques for protein purification. The substantially pure polypeptide will yield a single major band on a non-reducing polyacrylamide gel. The purity of an EPLIN polypeptide can also be determined by amino-terminal amino acid sequence analysis.

Isolated proteins of the present invention, preferably EPLIN proteins, include those proteins that have an amino acid sequence sufficiently homologous to the amino acid sequence of SEQ ID NO:2, SEQ ID NO:4, SEQ ID NO:10, SEQ ID NO:12, or SEQ ID NO:14, or are encoded by a nucleotide sequence sufficiently homologous to SEQ ID NO:1, SEQ ID NO:3, SEQ ID NO:9, SEQ ID NO:11, SEQ ID NO:13. As used herein, the term “sufficiently homologous” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least 30%, 40%, or 50% homology, preferably 60% homology, more preferably 70%-80%, and even more preferably 90-95% homology across the amino acid sequences of the domains and contain at least one and preferably two structural domains or motifs, are defined herein as sufficiently homologous. Furthermore, amino acid or nucleotide sequences which share at least 30%, 40%, or 50%, preferably 60%, more preferably 70-80%, or 90-95% homology and share a common functional activity are defined herein as sufficiently homologous.

One aspect of the invention pertains to isolated EPLIN proteins, and biologically active portions thereof, as well as polypeptide fragments suitable for use as immunogens to raise anti-antibodies. In one embodiment, native EPLIN proteins can be isolated from cells or tissue sources by an appropriate purification scheme using standard protein purification techniques. In another embodiment, EPLIN proteins are produced by recombinant DNA techniques. Alternative to recombinant expression, a EPLIN protein or polypeptide can be synthesized chemically using standard peptide synthesis techniques. Biologically active portions of a EPLIN protein include peptides comprising amino acid sequences sufficiently homologous to or derived from the amino acid sequence of the EPLIN protein, e.g., the amino acid sequence shown in SEQ ID NO:2, SEQ ID NO:4, SEQ ID NO:10, SEQ ID NO:12, or SEQ ID NO:14, which include less amino acids than the full length proteins, and exhibit at least one activity of a protein. Typically, biologically active portions comprise a domain or motif with at least one activity of the EPLIN protein. A biologically active portion of a EPLIN protein can be a polypeptide which is, for example, at least 10, 25, 50, 100 or more amino acids in length.

Thus, in a preferred embodiment, the EPLIN protein has an amino acid sequence shown in SEQ ID NO:2, SEQ ID NO:4, SEQ ID NO:10, SEQ ID NO:12, or SEQ ID NO:14. In other embodiments, the EPLIN protein is substantially homologous to SEQ ID NO:2, SEQ ID NO:4, SEQ ID NO:10, SEQ ID NO:12, or SEQ ID NO:14, and retains the functional activity of the protein of SEQ ID NO:2, SEQ ID NO:4, SEQ ID NO:10, SEQ ID NO:12, or SEQ ID NO:14, yet differs in amino acid sequence due to natural allelic variation or mutagenesis, as described in detail above. Accordingly, in another embodiment, the EPLIN protein is a protein which comprises an amino acid sequence at least about 41%, 42%, 45%, 50%, 55%, 59%, 60%, 65%, 70%, 75%, 80%, 81%, 85%, 90%, 95%, 98% or more homologous to the amino acid sequence of SEQ ID NO:2, SEQ ID NO:4, SEQ ID NO:10, SEQ ID NO:12, or SEQ ID NO:14 (e.g., the entire amino acid sequence of SEQ ID NO:2, SEQ ID NO:4, SEQ ID NO:10, SEQ ID NO:12, or SEQ ID NO:14).

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The invention includes functional polypeptides of EPLIN-α and EPLIN-β, and functional fragments thereof. As used herein, the term “functional polypeptide” refers to a polypeptide which possesses a biological function or activity which is identified through a defined functional assay and which is associated with a particular biologic, morphologic, or phenotypic alteration in the cell. Functional fragments of the EPLIN polypeptide, includes fragments of EPLIN as long as the activity, e.g., tumor suppressor activity, of EPLIN remains. Smaller peptides containing the biological activity of EPLIN are included in the invention. The biological function, for example, can vary from a polypeptide fragment as small as an epitope to which an antibody molecule can bind to a large polypeptide that is capable of participating in the characteristic induction or programming of phenotypic changes within a cell. A “functional polynucleotide” denotes a polynucleotide that encodes a functional polypeptide as described herein.

In addition, libraries of fragments of a EPLIN protein coding sequence can be used to generate a variegated population of EPLIN fragments for screening and subsequent selection of variants of a EPLIN protein. In one embodiment, a library of coding sequence fragments can be generated by treating a double stranded PCR fragment of a EPLIN coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the EPLIN protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of EPLIN proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique which enhances the frequency of functional mutants in the libraries, can be used in combination with the screening assays to identify EPLIN variants (Arkin and Yourvan (1992) Proc. Natl. Acad. Sci. USA 89:7811-7815; Delgrave et al. (1993) Protein Engineering 6(3):327-331).

In one embodiment, cell based assays can be exploited to analyze a variegated EPLIN library. For example, a library of expression vectors can be transfected into a cell line which ordinarily synthesizes and secretes EPLIN. The transfected cells are then cultured such that EPLIN and a particular mutant EPLIN are secreted and the effect of expression of the mutant on activity in cell supernatants can be detected, e.g., by any of a number of enzymatic assays. Plasmid DNA can then be recovered from the cells which score for inhibition, or alternatively, potentiation of EPLIN activity, and the individual clones further characterized.

As previously noted, minor modifications of the EPLIN primary amino acid sequence may result in proteins that have substantially equivalent activity as compared to the EPLIN polypeptide described herein. Such modifications may be deliberate, as by site-directed mutagenesis, or may be spontaneous. All of the polypeptides produced by these modifications are included herein as long as the tumor suppressor activity of EPLIN is present. Further, deletion of one or more amino acids can also result in a modification of the structure of the resultant molecule without significantly altering its activity. This can lead to the development of a smaller active molecule that would have broader utility. For example, it is possible to remove amino or carboxy terminal amino acids that may not be required for EPLIN activity.

In another aspect, the invention provides EPLIN chimeric or fusion proteins. As used herein, an EPLIN “chimeric protein” or “fusion protein” includes an EPLIN polypeptide linked to a non-EPLIN polypeptide. A “non-EPLIN polypeptide” refers to a polypeptide having an amino acid sequence corresponding to a protein that is not substantially homologous to the EPLIN protein, e.g., a protein that is different from the EPLIN protein and that is derived from the same or a different organism. The EPLIN polypeptide of the fusion protein can correspond to all or a portion e.g., a fragment described herein of an EPLIN amino acid sequence. In a preferred embodiment, an EPLIN fusion protein includes at least one (e.g. two) biologically active portion of an EPLIN protein. The non-EPLIN polypeptide can be fused to the N-terminus or C-terminus of an EPLIN polypeptide.

The fusion protein can include a moiety that has high affinity for a ligand. For example, the fusion protein can be a GST-EPLIN fusion protein in which the EPLIN sequences are fused to the C-terminus of the GST sequences. Such fusion proteins can facilitate the purification of recombinant EPLIN. Alternatively, the fusion protein can be an EPLIN protein containing a heterologous signal sequence at its N-terminus. In certain host cells (e.g., mammalian host cells), expression and/or secretion of EPLIN can be increased through use of a heterologous signal sequence.

Fusion proteins can include all or a part of a serum protein, e.g., an IgG constant region, or human serum albumin.

In another embodiment, the EPLIN nucleic acid molecules, proteins, and anti-antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

For example, the EPLIN fusion proteins of the invention can be incorporated into pharmaceutical compositions and administered to a subject in vivo. The EPLIN fusion proteins can be used to affect the bioavailability of an EPLIN substrate. EPLIN fusion proteins may be useful therapeutically for the treatment of disorders caused by, for example: (i) aberrant modification or mutation of a gene encoding an EPLIN protein; (ii) mis-regulation of the EPLIN gene; and (iii) aberrant post-translational modification of an EPLIN protein.

Moreover, EPLIN-fusion proteins of the invention can be used as immunogens to produce anti-EPLIN antibodies in a subject, to purify EPLIN ligands, and in screening assays to identify molecules that inhibit the interaction of EPLIN with an EPLIN substrate.

Expression vectors are commercially available that already encode a fusion moiety (e.g., a GST polypeptide). An EPLIN-encoding nucleic acid can be cloned into such an expression vector such that the fusion moiety is linked in-frame to the EPLIN protein.

In another aspect, the invention features a variant of an EPLIN polypeptide, e.g., a polypeptide that functions as an agonist (mimetic) or as an antagonist of EPLIN activities. Variants of the EPLIN proteins can be generated by mutagenesis, e.g., discrete point mutations, the insertion or deletion of sequences or the truncation of an EPLIN protein. An agonist of the EPLIN protein retains substantially the same, or a subset, of the biological activities of the naturally occurring form of an EPLIN protein. An antagonist of an EPLIN protein can inhibit one or more of the activities of the naturally occurring form of the EPLIN protein by, for example, competitively modulating an EPLIN-mediated activity of an EPLIN protein. Thus, specific biological effects can be elicited by treatment with a variant of limited function. Preferably, treatment of a subject with a variant having a subset of the biological activities of the naturally occurring form of the protein has fewer side effects in a subject relative to treatment with the naturally occurring form of the EPLIN protein.

Variants of an EPLIN protein can be identified by screening combinatorial libraries of mutants, e.g., truncation mutants, of an EPLIN protein for agonist or antagonist activity.

Libraries of fragments e.g., N terminal, C terminal, or internal fragments, of an EPLIN protein coding sequence can be used to generate a variegated population of fragments for screening and subsequent selection of variants of an EPLIN protein.

Variants in which a cysteine residue is added or deleted or in which a residue that is glycosylated is added or deleted are particularly preferred.

Methods for screening gene products of combinatorial libraries made by point mutations or truncation, and for screening cDNA libraries for gene products having a selected property are known. Recursive ensemble mutagenesis (REM), a new technique which enhances the frequency of functional mutants in the libraries, can be used in combination with screening assays to identify EPLIN variants (Arkin and Yourvan (1992) Proc. Natl. Acad. Sci. USA, 89:7811-7815; Delgrave et al. (1993) Protein Engineering, 6(3):327-331).

Cell based assays can be exploited to analyze a variegated EPLIN library. For example, a library of expression vectors can be transfected into a cell line, e.g., a cell line, which ordinarily responds to EPLIN in a substrate-dependent manner. The transfected cells are then contacted with EPLIN and the effect of the expression of the mutant on signaling by the EPLIN substrate can be detected, e.g., by measuring tumor suppressor activity in an appropriate assay. Plasmid DNA can then be recovered from the cells that score for inhibition, or alternatively, potentiation of signaling by the EPLIN substrate, and the individual clones further characterized.

In another aspect, the invention features a method of making an EPLIN polypeptide, e.g., a peptide having a non-wild type activity, e.g., an antagonist, agonist, or super agonist of a naturally occurring EPLIN polypeptide, e.g., a naturally occurring EPLIN polypeptide. The method includes: altering the sequence of an EPLIN polypeptide, e.g. by substitution or deletion of one or more residues of a non-conserved region, a domain, or residue disclosed herein, and testing the altered polypeptide for the desired activity.

In another aspect, the invention features a method of making a fragment or analog of an EPLIN polypeptide that retains at least one biological activity of a naturally occurring EPLIN polypeptide. The method includes: altering the sequence, e.g., by substitution or deletion of one or more residues, of an EPLIN polypeptide, e.g., altering the sequence of a non-conserved region, or a domain or residue described herein, and testing the altered polypeptide for the desired activity.

Screening Method

EPLIN nucleic acids, proteins, and derivatives of the present invention also have uses in screening assays to detect molecules that specifically bind to EPLIN nucleic acids, proteins, or derivatives and thus have potential use as agonists or antagonists of EPLIN, in particular, molecules that affect cell proliferation. In one embodiment, such assays are performed to screen for molecules with potential utility as anti-cancer drugs or lead compounds for drug development. The invention provides assays to detect molecules that specifically bind to EPLIN nucleic acids, proteins, or derivatives. For example, recombinant cells expressing EPLIN nucleic acids can be used to recombinantly produce EPLIN proteins in these assays, to screen for molecules that bind to an EPLIN protein. Molecules (e.g., putative binding partners of EPLIN) are contacted with the EPLIN protein (or fragment thereof) under conditions conducive to binding, and then molecules that specifically bind to the EPLIN protein are identified. Similar methods can be used to screen for molecules that bind to EPLIN derivatives or nucleic acids. Methods that can be used to carry out the foregoing are commonly known in the art.

By way of example, diversity libraries, such as random or combinatorial peptide or nonpeptide libraries can be screened for molecules that specifically bind to EPLIN. Many libraries are known in the art that can be used, e.g., chemically synthesized libraries, recombinant (e.g., phage display libraries), and in vitro translation-based libraries.

For example, agonists and antagonists of EPLIN can be identified using “biochip” technology. “Biochips” or arrays of binding agents, such as oligonucleotides and peptides, have become an increasingly important tool in the biotechnology industry and related fields.

These binding agent arrays, in which a plurality of binding agents are deposited onto a solid support surface in the form of an array or pattern, find use in a variety of applications, including drug screening, nucleic acid sequencing, mutation analysis, and the like. One important use of biochips is in the analysis of differential gene expression, where the expression of genes in different cells, normally a cell of interest and a control, is compared and any discrepancies in expression are identified. In such assays, the presence of discrepancies indicates a difference in the classes of genes expressed in the cells being compared.

In methods of differential gene expression, arrays find use by serving as a substrate to which is bound polynucleotide “probe” fragments. One then obtains “targets” from analogous cells, tissues, or organs of a healthy and diseased organism. The targets are then hybridized to the immobilized set of polynucleotide “probe” fragments. Differences between the resultant hybridization patterns are then detected and related to differences in gene expression in the two sources. Thus, the present invention provides nucleic acid and amino acid sequences useful for screening for differential expression of EPLIN in a cell.

The invention includes antibodies immunoreactive with EPLIN polypeptide or functional fragments thereof. Antibody which consists essentially of pooled monoclonal antibodies with different epitopic specificities, as well as distinct monoclonal antibody preparations are provided. Monoclonal antibodies are made from antigen containing fragments of the protein by methods well known to those skilled in the art (Kohler et al., Nature, 256:495, 1975). The term antibody as used in this invention is meant to include intact molecules as well as fragments thereof, such as Fab and F(ab′)₂, which are capable of binding an epitopic determinant on EPLIN.

Monoclonal antibodies used in the method of the invention are suited for use, for example, in immunoassays in which they can be utilized in liquid phase or bound to a solid phase carrier. In addition, the monoclonal antibodies in these immunoassays can be detectably labeled in various ways. Examples of types of immunoassays which can utilize monoclonal antibodies of the invention are competitive and non-competitive immunoassays in either a direct or indirect format. Examples of such immunoassays are the radioimmunoassay (RIA) and the sandwich (immunometric) assay. Detection of the antigens using the monoclonal antibodies of the invention can be done utilizing immunoassays which are run in either the forward, reverse, or simultaneous modes, including immunohistochemical assays on physiological samples. Those of skill in the art will know, or can readily discern, other immunoassay formats without undue experimentation.

Diagnostic Uses of EPLIN

EPLIN proteins, analogues, derivatives, and subsequences thereof, EPLIN nucleic acids (and sequences complementary thereto), anti-EPLIN antibodies, have uses in diagnostics. Such molecules can be used in assays, such as immunoassays, to detect, prognose, diagnose, or monitor various conditions, diseases, and disorders affecting EPLIN expression, or monitor the treatment thereof. In particular, such an immunoassay is carried out by a method comprising contacting a sample derived from a patient with an anti-EPLIN antibody under conditions such that immunospecific binding can occur, and detecting or measuring the amount of any immunospecific binding by the antibody. In a specific aspect, such binding of antibody, in tissue sections, can be used to detect aberrant EPLIN localization or aberrant (e.g., low or absent) levels of EPLIN. In a specific embodiment, antibody to EPLIN can be used to assay in a patient tissue or serum sample for the presence of EPLIN where an aberrant level of EPLIN is an indication of a diseased condition. By “aberrant levels,” is meant increased or decreased levels relative to that present, or a standard level representing that present, in an analogous sample from a portion of the body or from a subject not having the disorder.

Thus, the invention provides a method of detecting a cell proliferative disorder in a sample from a subject by contacting a first sample having, or suspected of having, a cell proliferative disorder with a reagent that binds to an EPLIN-specific cell component and detecting binding of the reagent to the component; contacting a second cell not having a cell proliferative disorder with a reagent that binds to an EPLIN-specific cell component and detecting binding of the reagent to the component; comparing the level of binding in the first sample with the level of binding in the second sample, wherein a decreased level of binding of the reagent to an EPLIN-specific cell component from the first sample is indicative of a cell proliferative disorder.

The term “cell proliferative disorder,” as used herein, refers to a condition characterized by abnormal cell growth. The condition can include both hypertrophic (the continual multiplication of cells resulting in an overgrowth of a cell population within a tissue) and hypotrophic (a lack or deficiency of cells within a tissue) cell growth or an excessive influx or migration of cells into an area of a body. The cell populations are not necessarily transformed, tumorigenic or malignant cells, but also can include normal cells. As used herein, an “EPLIN-specific cell component” includes, but is not limited to, RNA and DNA encoding an EPLIN protein, the EPLIN protein and fragments thereof, and EPLIN variants including translocations in EPLIN nucleic acids, truncations in the EPLIN gene or protein, changes in nucleotide or amino acid sequence relative to wild-type EPLIN.

Thus, the EPLIN molecules can act as novel diagnostic targets and therapeutic agents for controlling one or more of cellular proliferative and/or differentiative disorders, disorders associated with bone metabolism, immune disorders, hematopoietic disorders, cardiovascular disorders, liver disorders, viral diseases, pain or metabolic disorders.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few.

EPLIN genes and related nucleic acid sequences and subsequences, including complementary sequences, can also be used in hybridization assays. EPLIN nucleic acid sequences, or subsequences thereof comprising about at least 8 nucleotides, can be used as hybridization probes.

Hybridization assays can be used to detect, prognose, diagnose, or monitor conditions, disorders, or disease states associated with aberrant changes in EPLIN expression and/or activity as described. In particular, such a hybridization assay is carried out by a method comprising contacting a sample containing nucleic acid with a nucleic acid probe capable of hybridizing to EPLIN DNA or RNA, under conditions such that hybridization can occur, and detecting or measuring any resulting hybridization.

In specific embodiments, diseases and disorders involving over-proliferation of cells can be diagnosed, or their suspected presence can be screened for, or a predisposition to develop such disorders can be detected, by detecting increased levels of EPLIN protein, EPLIN RNA, or EPLIN functional activity or by detecting mutations in EPLIN RNA, DNA or protein (e.g., translocations in EPLIN nucleic acids, truncations in the EPLIN gene or protein, changes in nucleotide or amino acid sequence relative to wild-type EPLIN) that cause increased expression or activity of EPLIN. By way of example, levels of EPLIN protein can be detected by immunoassay, levels of EPLIN RNA can be detected by hybridization assays (e.g., Northern blots, dot blots), translocations and point mutations in EPLIN nucleic acids can be detected by Southern blotting, RFLP analysis, PCR using primers that preferably generate a fragment spanning at least most of the EPLIN gene, sequencing of the EPLIN genomic DNA or cDNA obtained from the patient.

In a preferred embodiment, levels of EPLIN mRNA or protein in a patient sample are detected or measured, in which increased levels indicate that the subject has, or has a predisposition to developing, a malignancy or hyperproliferative disorder; in which the increased levels are relative to the levels present in an analogous sample from a portion of the body or from a subject not having the malignancy or hyperproliferative disorder, as the case may be.

In another specific embodiment, diseases and disorders involving a deficiency in cell proliferation or in which cell proliferation is desirable for treatment, are diagnosed, or their suspected presence can be screened for, or a predisposition to develop such disorders can be detected, by detecting decreased levels of EPLIN protein, EPLIN RNA, or EPLIN functional activity, or by detecting mutations in EPLIN RNA, DNA or protein (e.g., translocations in EPLIN nucleic acids, truncations in the gene or protein, changes in nucleotide or amino acid sequence relative to wild-type EPLIN) that cause decreased expression or activity of EPLIN. By way of example, levels of EPLIN protein, levels of EPLIN RNA, EPLIN binding activity, and the presence of translocations or point mutations can be determined as described.

In a specific embodiment, levels of EPLIN mRNA or protein in a patient sample are detected or measured, in which decreased levels indicate that the subject has, or has a predisposition to developing, a malignancy or hyperproliferative disorder; in which the decreased levels are relative to the levels present in an analogous sample from a portion of the body or from a subject not having the malignancy or hyperproliferative disorder, as the case may be.

In using a monoclonal antibody for the in vivo detection of antigen, the detectably labeled monoclonal antibody is given in a dose that is diagnostically effective. The term “diagnostically effective” means that the amount of detectably labeled monoclonal antibody is administered in sufficient quantity to enable detection of the site having the EPLIN antigen for which the monoclonal antibodies are specific. The concentration of detectably labeled monoclonal antibody which is administered should be sufficient such that the binding to those cells having EPLIN is detectable compared to the background. Further, it is desirable that the detectably labeled monoclonal antibody be rapidly cleared from the circulatory system in order to give the best target-to-background signal ratio.

As a rule, the dosage of detectably labeled monoclonal antibody for in vivo diagnosis will vary depending on such factors as age, sex, and extent of disease of the individual. The dosage of monoclonal antibody can vary from about 0.001 mg/m² to about 500 mg/m², preferably 0.1 mg/m² to about 200 mg/m², most preferably about 0.1 mg/m² to about 10 mg/m². Such dosages may vary, for example, depending on whether multiple injections are given, tumor burden, and other factors known to those of skill in the art.

For in vivo diagnostic imaging, the type of detection instrument available is a major factor in selecting a given radioisotope. The radioisotope chosen must have a type of decay that is detectable for a given type of instrument. Still another important factor in selecting a radioisotope for in vivo diagnosis is that the half-life of the radioisotope be long enough so that it is still detectable at the time of maximum uptake by the target, but short enough so that deleterious radiation with respect to the host is minimized. Ideally, a radioisotope used for in vivo imaging will lack a particle emission, but produce a large number of photons in the 140-250 keV range, which may be readily detected by conventional gamma cameras.

For in vivo diagnosis, radioisotopes may be bound to immunoglobulin, either directly or indirectly, by using an intermediate functional group. Intermediate functional groups which often are used to bind radioisotopes that exist as metallic ions to immunoglobulins are the bifunctional chelating agents such as diethylenetriaminepentacetic acid (DTPA) and ethylenediaminetetraacetic acid (EDTA) and similar molecules. Typical examples of metallic ions that can be bound to the monoclonal antibodies of the invention are ¹¹¹In, ⁹⁷Ru, ⁶⁷Ga, ⁶⁸Ga, ⁷²As, ⁸⁹Zr, and ²⁰¹Tl.

A monoclonal antibody useful in the method of the invention can also be labeled with a paramagnetic isotope for purposes of in vivo diagnosis, as in magnetic resonance imaging (MRI) or electron spin resonance (ESR). In general, any conventional method for visualizing diagnostic imaging can be utilized. Usually gamma and positron emitting radioisotopes are used for camera imaging and paramagnetic isotopes for MRI. Elements that are particularly useful in such techniques include ¹⁵⁷Gd, ⁵⁵Mn, ¹⁶²Dy, ⁵²Cr, and ⁵⁶Fe.

Kits for Detection of EPLIN

The materials for use in the method of the invention are ideally suited for the preparation of a kit. Such a kit may comprise a carrier means being compartmentalized to receive one or more container means such as vials, tubes, and the like, each of the container means comprising one of the separate elements to be used in the method. For example, one of the container means may comprise an EPLIN or EPLIN binding reagent, such as an antibody or nucleic acid, respectively. The constituents may be present in liquid or lyophilized form, as desired. Thus, the present invention also provides a kit useful for the detection of an EPLIN-specific cell component, the kit comprising carrier means containing one or more containers comprising a first container containing an EPLIN-specific binding reagent. As used herein, an “EPLIN-specific binding reagent” includes nucleic acids, such as probes, which hybridize to an EPLIN-specific cell component, such as DNA or RNA encoding the EPLIN protein. An EPLIN-specific binding reagent also includes proteins, such as antibodies, which bind to an EPLIN protein or fragment or derivative thereof. It is understood that an EPLIN-specific binding reagent includes any molecule that binds to an EPLIN-specific cell component such that the component can be identified.

One of the container means may comprise a probe that is or can be detectably labeled. Such probe may be an antibody or nucleotide specific for a target protein, or fragments thereof, or a target nucleic acid, or fragment thereof, respectively, wherein the target is indicative, or correlates with, the presence of EPLIN protein or EPLIN transcript. For example, oligonucleotide probes of the present invention can be included in a kit and used for examining the presence of EPLIN nucleic acid, as well as the quantitative (relative) degree of binding of the probe for determining the lack of binding (hybridizing) to the sequences, thus indicating the likelihood for an subject having a cell proliferation-associated pathology, such as, for example, cancer.

Where the kit utilizes nucleic acid hybridization to detect the target nucleic acid, the kit may also have containers containing nucleotide(s) for amplification of the target nucleic acid sequence. When it is desirable to amplify the target nucleic acid sequence, such as an EPLIN nucleic acid sequence, this can be accomplished using oligonucleotide(s) that are primers for amplification. For example, the kit may contain reagents necessary to perform RT-PCR on a sample containing, or suspected of containing, a cell harboring a pathogenic lentivirus such as HIV. Oligonucleotide primers based upon identification of the flanking regions contiguous with the target nucleotide sequence can be included in the kit such that the primers bind to an EPLIN transcript in the presence of, and under conditions that promote RT-PCR. The level of EPLIN transcript in a sample can be quantitated by means known to those of skill in the art.

The method of the invention provides the basis for a kit useful for the detection, or lack thereof, of a target EPLIN nucleic acid sequence in a sample obtained from a subject having, or suspected of having, a neoplasia. The absence, or under-production of, EPLIN transcript obtained from such a sample is indicative of the presence of a neoplasia. The kit includes a carrier means being compartmentalized to receive therein one or more containers. For example, a first container contains nucleic acid primers which hybridize to the target nucleic acid (e.g., EPLIN RNA) for the purpose of performing semi-quantitative RT-PCR. In addition, the kit can provide a nucleic acid probe for detection of an EPLIN RNA transcript. Thus, a first container contains a nucleic acid hybridization probe that hybridizes to the target nucleic acid. Other target nucleic acid sequences of EPLIN can be determined by those of skill in the art. In addition, the kit may include a second container containing a means for detecting hybridization of the probe with the target nucleic acid. Such reporter means include a biotin-binding protein, such as avidin or streptavidin, bound to a reporter molecule, such as an enzymatic, fluorescent, or radionuclide label. Other reporter means and labels are well known in the art. The kit may also include an amplification polymerase and deoxyribonucleotide(s). The kit may further include nucleic acid amplification buffer. Preferably, the reagent that modifies unmethylated cytosine is bisulfite. The kit of the invention is intended to provide the reagents necessary to perform nucleic acid hybridization analysis as described herein.

Techniques for obtaining a sample containing, or believed to contain, neoplastic cells are usually based on collection of tissues containing such cells. Such tissue can include, for example, blood, lymph or other tissue. However, it is understood that the method of the invention is useful for detecting a neoplasia in any sample believed to contain such cells. Sample acquisition can be accomplished by any means that allows for the isolation of a sample from a subject that results in a sufficient quantity of fluid being obtained for testing.

The kit may also include a container containing antibodies that bind to a target protein, or fragments thereof. Thus, it is envisioned that antibodies that bind to EPLIN, or fragments thereof, can be included in a kit. In addition, the kit may include a second container containing a means for detecting binding of the antibody with the target EPLIN protein, or fragment thereof. Such reporter means include a biotin-binding protein, such as avidin or streptavidin, bound to a reporter molecule, such as an enzymatic, fluorescent, or radionuclide label. Other reporter means and labels are well known in the art.

Gene Therapy Methods

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system. For example, tt has been observed that certain tumor cells return to normal function when fused with normal cells, suggesting that replacement of a missing factor, such as a wild-type tumor suppressor gene expression product may serve to restore a tumor cell to a normal state. These observations have led to research aimed at providing genetic treatment of tumor cells having defective tumor suppressor genes. Thus, in another aspect, the invention provides a method for converting a neoplastic cell to a non-neoplastic state through the expression of wild-type levels of EPLIN.

Any of the methods known to the art for the insertion of DNA fragments into a vector, as described, for example, in Maniatis, T, Fritsch, E. F., and Sambrook, J. (1989): Molecular Cloning (a Laboratory manual), Cold Spring Harbor Laboratory, Cold Spring Harbor, N.Y.; and Ausubel, F. M., Brent, R., Kingston, R. E., Moore, D. D., Seidman, J. G., Smith, J. a., and Struhl, K. (1992): Current Protocols in Molecular Biology, John Wiley & Sons, New York, may be used to construct EPLIN encoding gene expression vectors consisting of appropriate transcriptional/translational control signals and the desired EPLIN cDNA sequence downstream from the first in-frame AUG codon. These methods may include in vitro DNA recombinant and synthetic techniques and in vivo genetic recombination. Expression of a nucleic acid sequence encoding EPLIN may be regulated by a second nucleic acid sequence so that EPLIN is expressed in a host infected or transfected with the recombinant DNA molecule. For example, expression of EPLIN may be controlled by any promoter/enhancer element known in the art. The promoter activation may be tissue specific or inducible by a metabolic product or administered substance.

Promoters/enhancers which may be used to control EPLIN gene expression include, but are not limited to, the native EPLIN promoter, the cytomegalovirus (CMV) promoter/enhancer (Karasuyama, H. et al., 1989, J. Exp. Med., 169:13), the human beta-actin promoter (Gunning, P. et al., 1987, Proc. Natl. Acad. Sci. USA, 84:4831), the glucocorticoid-inducible promoter present in the mouse mammary tumor virus long terminal repeat (HHTV LTR) (Klessig, D. F. et al., 1984, Mol. Cell Biol., 4:1354), the long terminal repeat sequences of Moloney murine leukemia virus (MULV LTR) (Weiss, R. et al., 1985, RNA Tumor Viruses, Cold Spring Harbor Laboratory, Cold Spring Harbor, N.Y.), the SV40 early region promoter (Bemoist and Chambon, 1981, Nature, 290:304), the promoter contained in the 3′ long terminal repeat of Rous sarcoma virus (RSV) (Yamamoto et al., 1980, Cell 22:787), the herpes simplex virus (HSV) thymidine kinase promoter/enhancer (Wagner et al., 1981, Proc. Natl. Acad. Sci. U.S.A., 78:1441), the regulatory sequences of the metallothionein gene (Brinster et al., 1982, Nature, 296:39), the adenovirus promoter (Yamada et al., 1985, Proc. Natl. Acad. Sci. U.S.A., 82:3567), and the herpes simplex virus LAT promoter (Wolfe, J. H. et al., 1992, Nature Genetics, 1:379).

Expression vectors compatible with mammalian host cells for use in genetic therapy of tumor or cancer cells, include, but are not limited to: plasmids, retroviral vectors, adenovirus vectors, herpes viral vectors, and non-replicative avipox viruses, as disclosed, for example, by U.S. Pat. No. 5,174,993, incorporated herein by reference.

Methods of administering viral vectors are well known. In general, the skilled artisan will appreciate that a retroviral vector, an adenovirus vector, a plasmid vector, or any other appropriate vector capable of expressing the EPLIN protein can be administered in vivo to a neoplastic cell by a wide variety of manipulations. All such manipulations have in common the goal of placing the vector in sufficient contact with the target tumor to permit the vector to transduce or transfect the tumor cells. Neoplastic cells present in the epithelial linings of hollow organs may be treated by infusing the vector suspension into a hollow fluid filled organ, or by spraying or misting into a hollow air filled organ. Thus, the tumor cell may be present in or among the epithelial tissue in the lining of pulmonary bronchial tree, the lining of the gastrointestinal tract, the lining of the female reproductive tract, genitourinary tract, bladder, the gall bladder and any other organ tissue accessible to contact with the vector.

The EPLIN encoding gene construct of the present invention may be placed by methods well known to the art into an expression vector such as a plasmid or viral expression vector. A plasmid expression vector may be introduced into a tumor cell by calcium phosphate transfection, liposome (for example, LIPOFECTIN)-mediated transfection, DEAE Dextran-mediated transfection, polybrene-mediated transfection, electroporation and any other method of introducing DNA into a cell.

A viral expression vector may be introduced into a target cell in an expressible form by infection or transduction. Such a viral vector includes, but is not limited to: a retrovirus, an adenovirus, a herpes virus and an avipox virus. When EPLIN is expressed in any abnormally proliferating cell, the cell replication cycle is arrested, thereby resulting in senescence and cell death and ultimately, reduction in the mass of the abnormal tissue, i.e., the tumor or cancer. A vector able to introduce the gene construct into a target cell and able to express EPLIN therein in cell proliferation-suppressing amounts can be administered by any effective method.

For example, a physiologically appropriate solution containing an effective concentration of active vectors can be administered topically, intraocularly, parenterally, orally, intranasally, intravenously, intramuscularly, subcutaneously or by any other effective means. In particular, the vector may be directly injected into a target cancer or tumor tissue by a needle in amounts effective to treat the tumor cells of the target tissue.

Alternatively, a cancer or tumor present in a body cavity such as in the eye, gastrointestinal tract, genitourinary tract (e.g., the urinary bladder), pulmonary and bronchial system and the like can receive a physiologically appropriate composition (e.g., a solution such as a saline or phosphate buffer, a suspension, or an emulsion, which is sterile except for the vector) containing an effective concentration of active vectors via direct injection with a needle or via a catheter or other delivery tube placed into the cancer or tumor afflicted hollow organ. Any effective imaging device such as X-ray, sonogram, or fiberoptic visualization system may be used to locate the target tissue and guide the needle or catheter tube.

In another alternative, a physiologically appropriate solution containing an effective concentration of active vectors can be administered systemically into the blood circulation to treat a cancer or tumor that cannot be directly reached or anatomically isolated.

In yet another alternative, target tumor or cancer cells can be treated by introducing EPLIN protein into the cells by any known method. For example, liposomes are artificial membrane vesicles that are available to deliver drugs, proteins and plasmid vectors both in vitro or in vivo (Mannino, R. J. et al., 1988, Biotechniques, 6:682) into target cells (Newton, A. C. and Huestis, W. H., Biochemistry, 1988, 27:4655; Tanswell, A. K. et al., 1990, Biochmica et Biophysica Acta, 1044:269; and Ceccoll, J. et al., Journal of Investigative Dermatology, 1989, 93:190). Thus, EPLIN protein can be encapsulated at high efficiency with liposome vesicles and delivered into mammalian cells in vitro or in vivo.

Liposome-encapsulated EPLIN protein may be administered topically, intraocularly, parenterally, intranasally, intratracheally, intrabronchially, intramuscularly, subcutaneously or by any other effective means at a dose efficacious to treat the abnormally proliferating cells of the target tissue. The liposomes may be administered in any physiologically appropriate composition containing an effective concentration of encapsulated EPLIN protein.

In one embodiment a tumor cell is transduced with a retrovirus vector, an adenovirus vector, a plasmid vector or any other appropriate vector capable of expressing the EPLIN protein in that tumor cell. The cancer cell may be present in a blood or bone marrow sample collected from a leukemia patient. A dose of EPLIN protein expressing retrovirus vector or adenovirus vector or plasmid vector or any other appropriate vector is administered to the sample of blood or bone marrow at a dose sufficient to transduce enough cells in the sample to produce a reduction in tumor cell numbers. The cell proliferation of the treated cancer cells will be slowed or terminated followed by a process similar to normal cellular differentiation or cell senescence. Analogously, blood or bone marrow or other tissue is treated ex vivo using an effective dose of a liposome-encapsulated EPLIN protein. Thereafter the sample may be returned to the donor or infused into another recipient.

Identification of Mouse and Zebrafish EPLIN

To identify conserved regions of EPLIN that may be important for its function, we have characterized the mouse (m) and zebrafish (zf) EPLIN. As in human, two isoforms, the 593 aa mEPLIN-α (77% identity; 83% similarity) and 753 aa mEPLIN-β (75% identity; 83% similarity), were present in mouse. mEPLIN-α is highly expressed in embryonic tissue and adult lung and spleen, whereas mEPLIN-β is preferentially expressed in kidney, testis, lung and liver. The analysis of mEPLIN gene revealed that the overall organization of the exons in mouse and human are conserved. In zebrafish, a 629 aa zfEPLIN polypeptide was identified corresponding to the mammalian EPLIN-β. Like its mammalian counterparts, ectopically expressed zfEPLIN co-localizes to the actin cytoskeleton. While the overall homology between mammalian and zebrafish EPLIN was not striking (37% identity; 50% similarity), there were seven highly conserved regions, which should be useful in structure-function studies of this novel protein.

The National Center for Biotechnology Information EST database was searched with the human EPLIN cDNA sequences to identify cDNA clones containing the mouse EPLIN (mEPLIN). Two mouse EST clones 1885402 and 1922179 that displayed a high degree of sequence homology to the 5′ regions of human EPLIN (hEPLIN) cDNA were selected for further analysis. When completely sequenced, these clones produced open reading frames of 593 aa and 753 aa, corresponding to mouse mEPLIN-α and -β. There were 7 amino acid sequence differences between the open reading frames of mouse EPLIN-α and -β. In particular, the sequence of EST clone 1885402 (mEPLIN-α) contained a T to A point transversion resulting in a premature TGA stop codon at amino acid 276. This stop codon is not present in the EST clone 1922179 (mEPLIN-β) or in the genomic DNA, indicating this mutation is unique to this particular EST clone.

A similar database search identified several zebrafish cDNA clones that displayed a high degree of translated amino acid sequence homology to the amino terminus of hEPLIN-β. One zebrafish EST clone (EST clone 2352091), when sequenced, generated an open reading frame of 629 aa, with a 50% sequence similarity (37% identity) to hEPLIN-β. Additional database searches using this cDNA sequence failed to produce zebrafish clones that would correspond to hEPLIN-α.

There is a high degree of sequence homology between human and mouse EPLIN (83% similarity) (FIG. 6). There were seven distinct regions of amino acid sequence similarity between hEPLIN-β and zfEPLIN. The region of most significant homology was at the LIM domain and the sequences immediately C-terminal to it (homology domain IV). The sequences amino-terminal to the LIM domain was punctuated by gaps in zfEPLIN, which serve to demarcate the homology domains I to III. Three additional segments of conserved regions (homology domains V to VII) were present in the region carboxyl-terminal to the LIM domain.

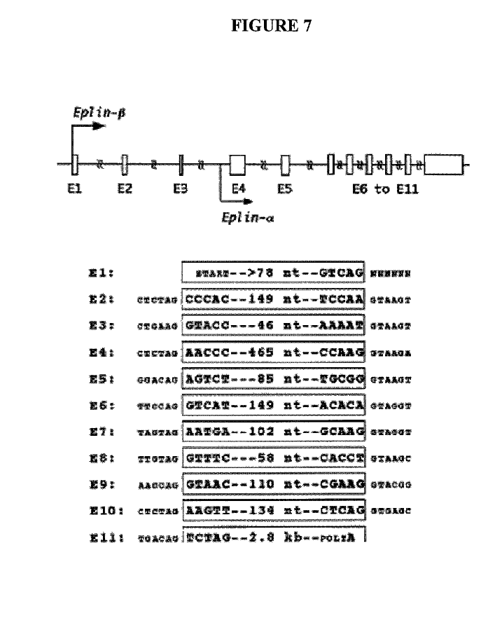

The 3′ region of mouse EPLIN-α cDNA (nt 1920-2243) was used as the probe to isolate the mouse EPLIN gene from a BAC genomic library. Using primers complementary to mouse EPLIN cDNA, the BAC clone RCPI22-121N19, which contains Exons 2 to 11 of the mouse EPLIN gene, was sequenced across the exon/intron boundaries (FIG. 7). The translated region of mEPLIN-β is encoded in 10 exons, which were all flanked by the conserved GT and AG dinucleotides. As in human, the translation of mEPLIN-α only required the Exons 4 to 11. The 5′ end of mEPLIN-α is located within the third intron, 114 nt upstream of the splice donor site of Exon 4.

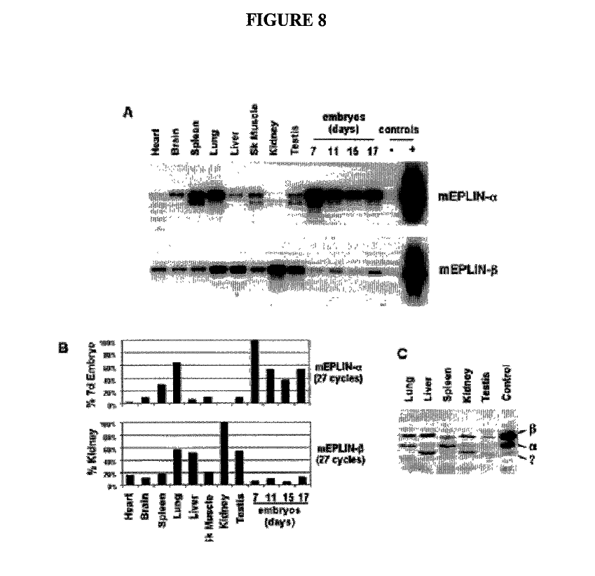

RT-PCR was utilized to identify mEPLIN transcripts and to distinguish mEPLIN-α transcripts from mEPLIN-β transcripts. A set of primers specific for either mEPLIN-α or -β and RNA prepared from various mouse tissues were used. mEPLIN-α was expressed in mouse embryos, followed by adult lung and spleen (FIG. 8A and B). mEPLIN-α expression in other adult tissues was considerably less. mEPLIN-β, on the other hand, was highly expressed in the kidney, lung, liver, and testis.

An immunoblot analysis using anti-EPLIN antisera was performed to complement the semi-quantitative RT-PCR assay (FIG. 8C). The ratio of mEPLIN-α to -β protein levels were consistent with the findings from the RT-PCR assays: mEPLIN-β was seen as the major form in the liver, kidney, and testis, while mEPLIN-α was the predominant form in the spleen. In the lung, both isoforms were present at an equivalent level. The immunoblot also detected a faster migrating band in the liver and kidney, suggesting the existence of additional tissue-specific EPLIN isoformns. The preferential expression of EPLIN-α and -β in a tissue specific manner suggests that each EPLIN isoform may provide a non-overlapping role in the development and maintenance of various epithelial organs.

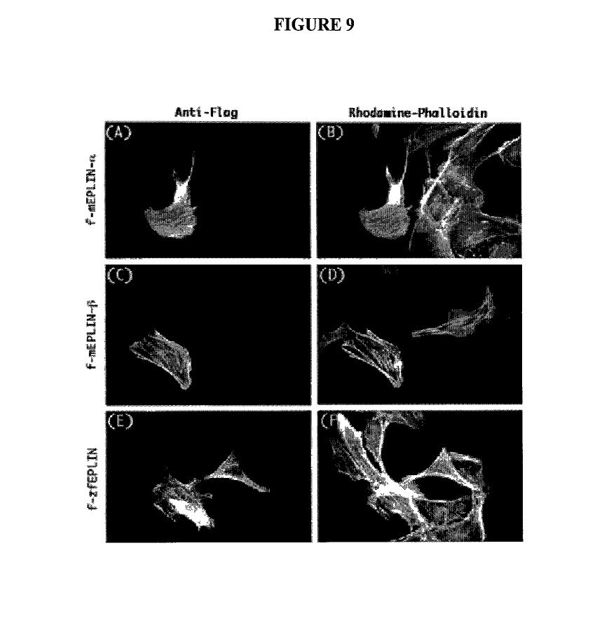

To confirm that the isolated zfEPLIN cDNA encodes a homologue of mammalian EPLIN, the subcellular localization of zfEPLIN was examined. Using the isolated mEPLIN and zfEPLIN cDNA, amino-terminal FLAG-epitope tagged mEPLIN-α, mEPLIN-β, and zfEPLIN were constructed. In the human Saos2 osteosarcoma cells, both mEPLIN-α and -β co-localized to the actin stress fibers following transient transfection (FIG. 9A and B). In ˜50% of the transfected cells, zfEPLIN was present along the actin filaments (FIG. 9C).

Identification of EPLIN Regulatory Sequences

The invention further provides an isolated, synthetic, or recombinant polynucleotide comprising a EPLIN non-coding regulatory sequence. In alternative embodiments, the promoter sequence comprises at least 15, 50, 100, 150, 200, 250, 500, 1000, 2500, 3500 or at least 4500 bases as set forth in SEQ ID NO:15 or SEQ ID NO:16. Other embodiments include sequences starting within about one to 5 nucleotides of a translation start codon and ending at about 50, 100, 150, 200, 250, 500, 1000, 2500 or 4500 nucleotides upstream of the translation start codon of the EPLIN coding sequence.

The invention provides an isolated, synthetic, or recombinant polynucleotide comprising a EPLIN non-coding regulatory sequence derived from a vertebrate source operably linked to a heterologous nucleic acid sequence. The invention is further provides compositions and methods for expressing a heterologous nucleic acid sequence operatively linked to a EPLIN non-coding regulatory sequence. For example, expression of a heterologous nucleic acid sequence can be regulated by the non-coding sequence of EPLIN-α protein (EPLIN) gene promoter region (GenBank Acc. No. AF245392) (SEQ ID NO:15), or EPLIN-β protein (EPLIN) gene promoter region (GenBank Acc. No. AF245391) (SEQ ID NO:16), or any fragment thereof capable of regulating transcription.

The invention further provides a method for screening for a compound that binds to a EPLIN non-coding regulatory sequence, such as a EPLIN-α or a EPLIN-β non-coding regulatory sequence, comprising: a) providing a isolated, synthetic, or recombinant polynucleotide comprising a EPLIN non-coding regulatory sequence and a test compound, b) contacting the polynucleotide with the test compound, and c) measuring the ability of the test compound to bind to the polynucleotide.

The invention also provides a method for a method for screening for a compound that modulates a EPLIN non-coding regulatory sequence, such as EPLIN-α or EPLIN-β non-coding regulatory sequence activity, comprising: a) providing a first polynucleotide comprising an isolated, synthetic, or recombinant EPLIN non-coding regulatory sequence operably associated to a heterologous nucleic acid sequence, and a test compound, b) contacting the first polynucleotide with the test compound, and c) measuring the ability of the test compound to modulate transcription of the heterologous nucleic acid sequence. The heterologous nucleic acid sequence can encode a protein. The protein can be detectable by fluorescence or phosphorescence or by virtue of its possessing an enzymatic activity. The detectable protein can be firefly luciferase, alpha-glucuronidase, alpha-galactosidase, chloramphenicol acetyl transferase, green fluorescent protein, enhanced green fluorescent protein, and the human secreted alkaline phosphatase.

The invention also provides a composition comprising an isolated nucleic acid molecule comprising a EPLIN non-coding regulatory sequence, wherein the non-coding regulatory sequence comprises about 100 to about 200, 200 to about 400, 400 to about 900, or 900 to about 2500, or 2500 to about 5000 nucleotides upstream of a transcriptional start site of an EPLIN coding sequence.

The invention also provides a vector comprising a EPLIN non-coding regulatory sequence operably linked to a heterologous nucleic acid sequence, wherein the non-coding regulatory sequence comprises about 100 to about 200, 200 to about 400, 400 to about 900, or 900 to about 2500, or 2500 to about 5000 nucleotides upstream of a transcriptional start site of an EPLIN coding sequence.

The invention provides a transformed cell comprising a EPLIN non-coding regulatory sequence operably linked to a heterologous nucleic acid sequence, wherein the non-coding regulatory sequence comprises about 100 to about 200, 200 to about 400, 400 to about 900, or 900 to about 2500, or 2500 to about 5000 nucleotides upstream of a transcriptional start of an EPLIN coding sequence. The heterologous sequence can code for a cellular toxin. The cellular toxin can be a Herpes virus thymidine kinase, ricin, abrin, diphtheria, gelonin, Pseudomonas exotoxin A, tumor necrosis factor alpha (TNF-alpha), Crotalus durissus terrificus toxin, Crotalus adamenteus toxin, Naja naja toxin, and Naja mocambique toxin.

The invention also provides a method of expressing a heterologous nucleic acid sequence in a cell comprising: a) transforming said cell with a vector or an expression cassette comprising a EPLIN non-coding regulatory sequence, wherein the non-coding regulatory sequence consists of about 100 to about 200, 200 to about 400, 400 to about 900, or 900 to about 2500, or 2500 to about 5000 nucleotides upstream of a transcriptional start site of an EPLIN coding sequence, and wherein the non-coding regulatory sequence is operably linked to the heterologous nucleic acid sequence; and b) growing said cell under conditions where the heterologous nucleic acid sequence is expressed in said cell.

The EPLIN promoter sequences of the invention and nucleic acids used to practice this invention, whether RNA, cDNA, genomic DNA, or hybrids thereof, may be isolated from a variety of sources, genetically engineered, amplified, and/or expressed recombinantly. Any recombinant expression system can be used, including, e.g., bacterial, yeast, insect or mammalian systems. Alternatively, these nucleic acids can be chemically synthesized in vitro. Techniques for the manipulation of nucleic acids, such as, e.g., subcloning into expression vectors, labeling probes, sequencing, and hybridization are well described in the scientific and patent literature, see e.g., ed., MOLECULAR CLONING: A LABORATORY MANUAL (2ND ED.), Vols. 1-3, Cold Spring Harbor Laboratory, (1989) (“Sambrook”); CURRENT PROTOCOLS IN MOLECULAR BIOLOGY, Ausubel, ed. John Wiley & Sons, Inc., New York (1997) (“Ausubel”); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993) (“Tijssen”). Nucleic acids can be analyzed and quantified by any of a number of general means well known to those of skill in the art. These include, e.g., analytical biochemical methods such as NMR, spectrophotometry, radiography, electrophoresis, capillary electrophoresis, high pressure liquid chromatography (HPLC), thin layer chromatography (TLC), and hyperdiffusion chromatography, various immunological methods, such as fluid or gel precipitin reactions, immunodiffusion (single or double), immunoelectrophoresis, radioimmunoassays (RIAs), enzyme-linked immunosorbent assays (ELISAs), immunofluorescent assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

The invention provides, for example, a EPLIN-α gene regulatory sequence encompassing a nucleic acid sequence that extends about 4.5 kb 5′ from the site of initiation of transcription of the EPLIN-α coding sequence. The invention further provides a EPLIN-α gene regulatory sequence encompassing a nucleic acid sequence that extends about 1.8 kb 5′ from the site of initiation of transcription of the EPLIN-α coding sequence. The invention also provides a EPLIN-α gene regulatory sequence encompassing a nucleic acid sequence that extends about 1.2 kb 5′ from the site of initiation of transcription of the EPLIN-α coding sequence. In addition, the invention provides a EPLIN-α gene regulatory sequence encompassing a nucleic acid sequence that extends about 0.7 kb 5′ from the site of initiation of transcription of the EPLIN-α coding sequence. The regulatory sequences include at least one serum response element (SRE) composed of a serum response factor (SRF) binding site (CCTTATAAGG) and neighboring Ets motifs (ATCCTG), located between nucleotides −57 and −89 of the EPLIN-α regulatory sequence. The SRE consists of the binding site for ubiquitous SRF and adjoining binding sites for ternary complex factors (TCF) of Ets family transcription, including Elk-1, Sap-1, and Sap-2/ERP/Net. The SRF can mediate transcriptional activation in response to serum, lysophosphatidic acids, or activation of Rho family small GTPases. The TCF is stimulated in response to activation of the Ras/Raf/MAP kinase pathway to form a ternary complex with SRF at the SRE.

The invention further provides a EPLIN-β gene regulatory sequence encompassing a nucleic acid sequence that extends to about 2.5 kb 5′ from the site of initiation of transcription of the EPLIN-α coding sequence and contains several Sp1 consensus sites.

The invention provides a nucleic acid molecule comprising non-coding regulatory elements derived from those sequences that regulate EPLIN mRNA transcription. The invention provides a nucleic acid construct the sequence of which comprises SEQ ID NO:15 operably associated to a heterologous coding sequence. In another aspect, the invention provides a nucleic acid construct the sequence of which comprises SEQ ID NO:16 operably associated to a heterologous coding sequence. In another aspect, the present invention provides vectors comprising the aforementioned nucleic acid constructs. The invention further provides a nucleic acid construct comprising at least one EPLIN non-coding regulatory sequence the sequence of which comprises SEQ ID NO:15 or 16, and a heterologous nucleic acid sequence operatively linked to the regulatory sequence, wherein expression of the heterologous sequence is regulated by the non-coding sequence.

Thus, a nucleic acid construct of the invention can include a regulatory sequence derived from the EPLIN-α or β gene. In the nucleic acid construct of the invention, the ATG start codon is typically provided by the nucleic acid sequence expressing the product of interest. As used herein, a “nucleic acid construct” of the invention includes at least one, or multiple, stress-responsive non-coding regulatory sequences and a heterologous nucleic acid sequence operatively linked to the regulatory sequence, wherein expression of the heterologous sequence is regulated by the non-coding sequence. A nucleic acid construct of the invention can be included in an expression vector. An “expression vector” refers to a plasmid, virus or other vehicle known in the art that has been manipulated by insertion or incorporation of the nucleic acid construct of the invention. The expression vector typically contains an origin of replication, as well as specific genes that allow phenotypic selection of the transformed cells. Vectors suitable for use in the present invention are well known in the art.

As used herein, the term “non-coding regulatory sequence” or “regulatory element” refers to a nucleic acid sequence capable of controlling the transcription of an operably associated gene. A non-coding regulatory sequence of the invention may include a promoter, an enhancer and/or a silencer, for example. Therefore, placing a gene under the regulatory control of a non-coding regulatory sequence or a regulatory element means positioning the gene such that the expression of the gene is controlled by the regulatory sequence(s).

A “promoter” is a nucleotide sequence that is capable of directing transcription in at least one context, e.g., when it is operably linked to a heterologous sequence in a plasmid within a plant cell. In other words, a promoter can exist without downstream sequences to transcribe, so long as the promoter sequence can direct transcription when placed upstream of a heterologous sequence in a different context. In general, promoters are found positioned 5′ (upstream) of the genes that they control. Thus, in the construction of promoter gene combinations, the promoter is preferably positioned upstream of the gene and at a distance from the transcription start site that approximates the distance between the promoter and the gene it controls in the natural setting. As is known in the art, some variation in this distance can be tolerated without loss of promoter function. Similarly, the preferred positioning of a regulatory element, such as an enhancer, with respect to a heterologous nucleic acid sequence placed under its control reflects its natural position relative to the structural gene it naturally regulates. Enhancers are believed to be relatively position and orientation independent in contrast to promoter elements. The noncoding sequences or intron sequences (e.g., which contain regulatory sequences) that are used in the invention construct are not more than about 9 kbp in length.

Regulatory sequence function during expression of a gene under its regulatory control and can be tested at the transcriptional stage using DNA/RNA and RNA/RNA hybridization assays (e.g., in situ hybridization, nucleic acid hybridization in solution or solid support) and at the translational stage using specific functional assays for the protein synthesized (e.g., by enzymatic activity, by immunoassay of the protein, by in vitro translation of mRNA or expression in microinjected xenopus oocytes).

The term “operably associated” refers to functional linkage between the regulatory sequence and the nucleic acid sequence regulated by the regulatory sequence. The operably linked regulatory sequence controls the expression of the product expressed by the nucleic acid sequence. Alternatively, the functional linkage also includes an enhancer element. “Promoter” means the minimal nucleotide sequence sufficient to direct transcription. Also included in the invention are those promoter elements that are sufficient to render promoter-dependent nucleic acid sequence expression controllable for cell-type specific, tissue specific, or inducible by external signals or agents; such elements may be located in the 5′ or 3′ regions of the native gene, or in the introns.

“Gene expression” or “nucleic acid sequence expression” means the process by which a nucleotide sequence undergoes successful transcription and translation such that detectable levels of the delivered nucleotide sequence are expressed in an amount and over a time period so that a functional biological effect is achieved. “Expressible genetic construct” as used herein means a construct that has a EPLIN regulatory sequence positioned with a heterologous nucleic acid sequence encoding a desired product, such that the nucleic acid sequence is expressed.

A “heterologous sequence” is a nucleotide sequence that is not naturally operably linked to the EPLIN promoter in a naturally occurring organism. A heterologous nucleic acid sequence of the invention can encode a “therapeutic agent” effective for treating, for example, a cell proliferative disorder or a disorder associated with glucose starvation, such as diabetes. As used herein, a “therapeutic agent” can include a structural gene that encodes a biologically active protein of interest. The term “structural gene” excludes the non-coding regulatory sequence that drives transcription. The structural gene may be derived in whole or in part from any source known to the art, including a plant, a fungus, an animal, a bacterial genome or episome, eukaryotic, nuclear or plasmid DNA, cDNA, viral DNA or chemically synthesized DNA. A structural gene may contain one or more modifications in either the coding or the untranslated regions which could affect the biological activity or the chemical structure of the expression product, the rate of expression or the manner of expression control. Such modifications include, but are not limited to, mutations, insertions, deletions and substitutions of one or more nucleotides. The structural gene may constitute an uninterrupted coding sequence or it may include one or more introns, bound by the appropriate splice junctions. The structural gene may also encode a fusion protein. It is contemplated that introduction into animal tissue of nucleic acid constructs of the invention will include constructions wherein the structural gene and its regulatory sequence are each derived from different animal species.

A structural gene can encode an enzyme, such as a drug-metabolizing enzyme that confers a dominant, negatively selectable phenotype to a cell, such as cell death. Such a gene can encode an enzyme that can convert a non-therapeutically effective compound in to a therapeutically effective compound. For example, the activation of a relatively nontoxic (i.e., non-therapeutically effective) prodrug to a cytotoxic (i.e., therapeutically effective) compound in a specifically targeted tissue can be used to effectively treat a cell proliferative disorder. Enzymes capable of performing such a function include herpes simplex virus (HSV) thymidine kinase, vesicular stomatitis virus (VSV) thymidine kinase, deoxycytidine kinase, cytosine deaminase or nucleoside phosphorylase. Prodrugs converted by the aforementioned enzymes include ganciclovir, acyclovir, 6-methoxypurine arabinoside (Ara-M), cytosine arabinoside or cytarabine (Ara-C), fludarabine, 2-chlorodeoxyadenosine, difluorodeoxycytidine, 5-fluorocytidine and 6-methylpurine-2′-deoxyriboside (MeP-dr).

A therapeutic agent of the invention also includes nucleic acid sequences that encode cell cycle blockers such as GATA-6 (Suzuki et al, Genomics, 38:283, 1996), anti-angiogenesis proteins such as endostatin and angistatin (Folkman J., Nature Med. 1:27, 1995), anti-sense gene sequences (Wang, Nature Med. 3:887, 1997), and viral subunit vaccines (Donnelly et al. Nature Med. 1:583, 1995).

A therapeutic agent also encompasses those sequences encoding proteins, such as asparaginase, that induce cell death by depriving a cell of a necessary metabolite. Asparaginase induces apoptosis by catalyzing the hydrolysis of circulating asparagine to aspartic acid and ammonia, thus depriving cells of the asparagine necessary for protein synthesis, leading to cell death.

A therapeutic agent of the invention also includes immunomodulators and other biological response modifiers. The term “biological response modifiers” encompasses substances that are involved in modifying the immune response in such manner as to enhance the destruction of tumor, for example. Examples of immune response modifiers include such compounds as lymphokines. Lymphokines include tumor necrosis factor, the interleukins, lymphotoxin, macrophage-activating factor, migration inhibition factor, colony stimulating factor, and interferon. Included in this category are immunopotentiating agents including nucleic acids encoding a number of the cytokines classified as “interleukins”. These include, for example, interleukins 1 through 12. Also included in this category, although not necessarily working according to the same mechanisms, are interferons, and in particular gamma interferon (γ-IFN), tumor necrosis factor (TNF) and granulocyte-macrophage-colony stimulating factor (GM-CSF). Nucleic acids encoding growth factors, toxic peptides, ligands, receptors, suicide factors (e.g., TK) or other physiologically important proteins can also be introduced into specific cells of the prostate.

Further, a therapeutic agent includes sense or antisense nucleic acids encoded by a heterogenous nucleic acid of the invention. For example, a sense polynucleotide sequence (the DNA coding strand) encoding a polypeptide can be introduced into the cell to increase expression of a “normal” gene. Other cell disorders can also be treated with nucleic acid sequences that interfere with expression at the translational level. This approach utilizes, for example, antisense nucleic acid, ribozymes, or triplex agents to block transcription or translation of a specific mRNA, either by masking that mRNA with an antisense nucleic acid or triplex agent, or by cleaving it with a ribozyme. Alternatively, the method includes administration of a reagent that mimics the action or effect of a gene product or blocks the action of the gene. Therefore, when a cell proliferative disorder, such as cancer, is etiologically linked with over expression of a polynucleotide, it would be desirable to administer an inhibiting reagent such as an antisense polynucleotide. For example, overexpression of the bcl-2 gene that is translocated in nodular non-Hodgkin's lymphomas, inactivates a key pathway of programmed cell death (apoptosis) and leads to continuous proliferation and survival of highly mutated tumor cells that have the capacity to survive DNA damage. Similarly, an increase in expression of the D cyclin (the prad oncogene) promotes cell entry into DNA synthesis. Additional oncogenes that promote cell proliferation include ABL, ERBB-1, ERBB-2 (NEU), GIP, GSP, MYC, L-MYC, N-MYC, H-RAS, RET, ROS, K-SAM, SIS, SRC, C-FOS, C-JUN AND TRK. Thus, efforts directed toward restoring apoptosis in tumor cells, by inhibiting the overexpression of an apoptosis inhibitor, such as bcl-2, or cell proliferation promoting oncogene, such as Ras, can be accomplished using antisense methodology.

The invention further provides a method of screening and isolating a EPLIN promoter binding compound by contacting a EPLIN promoter sequence of the invention (particularly, an identified cis-acting regulatory sequence) with a test compound and measuring the ability of the test compound to bind the selected nucleic acid. The test compound, as discussed above, can be any agent capable of specifically binding to a EPLIN promoter activity, including compounds available in chemical (e.g., combinatorial) libraries, a cell extract, a nuclear extract, a protein or peptide.

A variety of well-known techniques can be used to identify polypeptides which specifically bind to EPLIN promoter, e.g., mobility shift DNA-binding assays, methylation and uracil interference assays, DNase and hydroxy radical footprinting analysis, fluorescence polarization, and UV crosslinking or chemical cross-linkers. For a general overview, see, e.g., Ausubel (chapter 12, DNA-Protein Interactions). One technique for isolating co-associating proteins, including nucleic acid and DNA/RNA binding proteins, includes use of UV crosslinking or chemical cross-linkers, including e.g., cleavable cross-linkers dithiobis (succinimidylpropionate) and 3,3′-dithiobis (sulfosuccinimidyl-propionate); see, e.g., McLaughlin (1996) Am. J. Hum. Genet. 59:561-569; Tang (1996) Biochemistry 35:8216-8225; Lingner (1996) Proc. Natl. Acad. Sci. USA 93:10712; Chodosh (1986) Mol. Cell. Biol 6:4723-4733. In many cases, there is a high likelihood that a specific protein (or a related protein) may bind to an EPLIN promoter sequence, e.g., a Myc, NF-kappa B, EF2, Sp1, AP-1 or CAAT box binding site. In these scenarios, where an antibody may already be available or one can be easily generated, co-immunoprecipitation analysis can be used to identify and isolate EPLIN promoter-binding, trans-acting factors. The trans-acting factor can be characterized by peptide sequence analysis. Once identified, the function of the protein can be confirmed by methods known in the art, for example, by competition experiments, factor depletion experiments using an antibody specific for the factor, or by competition with a mutant factor.

Alternatively, EPLIN promoter-affinity columns can be generated to screen for potential EPLIN binding proteins. In a variation of this assay, EPLIN promoter subsequences are biotinylated, reacted with a solution suspected of containing a binding protein, and then reacted with a strepavidin affinity column to isolate the nucleic acid or binding protein complex (see, e.g., Grabowski (1986) Science 233:1294-1299; Chodosh (1986) supra). The promoter-binding protein can then be conventionally eluted and isolated. Mobility shift DNA-protein binding assay using nondenaturing polyacrylamide gel electrophoresis (PAGE) is an extremely rapid and sensitive method for detecting specific polypeptide binding to DNA (see, e.g., Chodosh (1986) supra, Carthew (1985) Cell 43:439-448; Trejo (1997) J. Biol. Chem. 272:27411-27421; Bayliss (1997) Nucleic Acids Res. 25:3984-3990).

Interference assays and DNase and hydroxy radical footprinting can be used to identify specific residues in the nucleic acid protein-binding site, see, e.g., Bi (1997) J. Biol. Chem. 272:26562-26572; Karaoglu (1991) Nucleic Acids Res. 19:5293-5300. Fluorescence polarization is a powerful technique for characterizing macromolecular associations and can provide equilibrium determinations of protein-DNA and protein-protein interactions. This technique is particularly useful (and better suited than electrophoretic methods) to study low affinity protein-protein interactions, see, e.g., Lundblad (1996) Mol. Endocrinol. 10:607-612.

Proteins identified by these techniques can be further separated on the basis of their size, net surface charge, hydrophobicity and affinity for ligands. In addition, antibodies raised against such proteins can be conjugated to column matrices and the proteins immunopurified. All of these general methods are well known in the art. See,.e.g, Scopes, R. K., Protein Purification: Principles and Practice, 2nd ed., Springer Verlag, (1987).

It is understood that the examples and embodiments described herein are for illustrative purposes only and that various modifications or changes in light thereof will be suggested to persons skilled in the art and are to be included within the spirit and purview of this application and scope of the appended claims.

### EXAMPLE 1

### Materials and Methods

Cell Cultures

Human mammary epithelial cells (MEC) and normal human dermal fibroblasts were purchased from Clonetics. Breast cancer cell lines HBL-100, BT-20, SK-Br-3 and T-47D cells were cultured in RPMI 1640 medium supplemented with 10% fetal bovine serum (FBS) with T-47D cells receiving 1×ITS supplement (Sigma). MCF-7 and MDA-MB-231 cells were cultured in DMEM supplemented with 10% FBS. BeWo cells were cultured in Ham's F12K medium supplemented with 15% FBS.

Northern Blot Analysis and cDNA Cloning

10 μg total RNA isolated from cell cultures using RNA STAT-60 (Tel-Test) was used in Northern analysis as previously described (Chang et al., Oncogene, 16:1921, 1998). Filter membranes were probed with cDNA clone #21 (corresponding to amino acids 268-462 of EPLIN-β) and hybridization signals were quantified on a phosphorimager (Molecular Dynamics). All probes were labeled with [³²P]-α-dCTP using a random prime labeling kit (Stratagene). Multiple tissue mRNA blots was purchased from Clontech and used in hybridization following the manufacturer's protocol. The cDNA insert from clone #21 was used as a probe to isolate full length EPLIN-α and -β cDNAs from a HeLa cell cDNA library. Two representative clones were fully sequenced to obtain approximately 3.6 kb of sequence.

Antibodies and Protein Analysis

The carboxy terminal region of EPLIN (aa 680-759 of EPLIN-β) was cloned into the pQE-30 vector (Qiagen) and expressed as a 6×His-tagged fusion protein in E. coli strain XL-1 Blue. The recombinant protein was purified on Ni-NTA agarose under native conditions following the manufacturer's recommendations and used as immunogen for polyclonal rabbit anti-EPLIN antibodies (Covance Research Products).

Cell lysates used in immunoblot analyses were prepared by boiling tissue culture cells or minced tissues in 0.2% SDS in TE (25 mM Tris-HCl, pH 7.5, 1 mM EDTA). 20 μg of cell lysates were separated by SDS-polyacrylamide gel electrophoresis and transferred to a nitrocellulose membrane. EPLIN isoforms were detected with polyclonal anti-EPLIN antibodies (1:10,000). To control the amounts of protein lysates, the filter membrane was also probed with a monoclonal anti-α-tubulin antibody (Sigma) at 1:2,000 dilution. Following incubation with a horseradish peroxidase-conjugated secondary antibody (Jackson ImmunoResearch), the immunoblots were developed using enhanced chemiluminescence (NEN).

Immunofluorescence

HOK18C and BeWo cells cultured on fibronectin-coated glass coverslips for 18 h were fixed in 3.7% formaldehyde (LADD Research) in PBS for 10 min and permeabilized in 0.2% Triton X-100 in PBS for 5 min. The slides were preincubated in a blocking buffer (0.1% Tween-20+10% goat serum in PBS) for 30 min before the addition of polyclonal anti-EPLIN antibodies (1:200 dilution). All incubations were performed at room temperature. HOK18C cells were labeled with Texas Red-conjugated goat anti-rabbit IgG secondary antibody (Jackson ImmunoResearch) and Oregon Green phalloidin (Molecular Probes) while BeWo cells were labeled with fluorescein isothiocyanate-conjugated goat anti-rabbit IgG secondary antibody (Jackson ImmunoResearch) and Texas Red phalloidin (Molecular Probes). Coverslips were mounted with ProLong Antifade (Molecular Probes) and viewed under a fluorescence microscope (Nikon). Pre-immune sera did not produce a staining pattern.

Conditional Expression of EPLIN

U2-OS cells were transfected with the plasmid pTet-On (Clontech) to create U2-OS Tet-On cells expressing tetracycline-inducible transactivator. EPLIN-α and -β cDNAs were cloned into the pTRE vector (Clontech) that has been modified by the insertion of an amino terminal FLAG epitope and multiple cloning sites. pTRE-FLAG-EPLIN-α or -β and pBABEpuro (Morgenstern and Land, Nucleic Acids Res., 18:587, 1990) plasmids were co-transfected into the U2-OS cells and the stable transfectants were selected with puromycin (1 mg/ml). The expression of EPLIN in stable cell lines was induced by the addition of 0.5 mg/ml doxycycline (Sigma). Morphological changes were observed 48 h after the induction of EPLIN. Cell growth was determined by a tetrazolium dye colorimetric assay (Denizot and Lang, J. Immunological Methods, 89:271, 1986) with the following modifications. Cells were incubated for 3.5 h in phenol red-free RPMI 1640 medium (Sigma) with 1 mg/ml MTT (3-[4,5-dimethylthiazol-2-y]-2,5-diphenyl tetrazolium bromid) (Sigma) and the formazan product was solubilized in isopropanol containing 0.04 N hydrochloric acid and 1% Triton X-100.

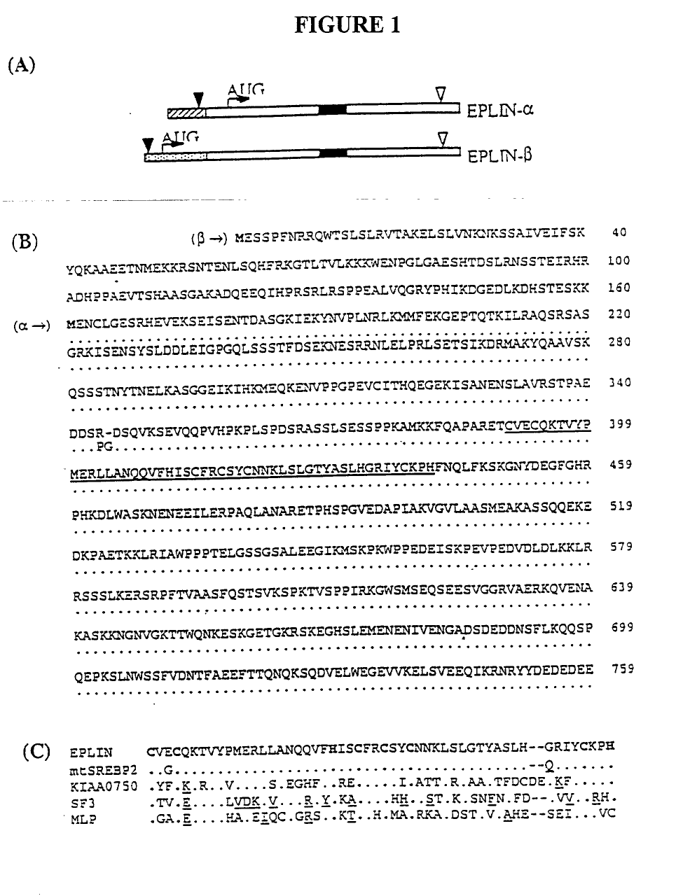

FIG. 1A is a schematic diagram of two EPLIN cDNAs. The sequence of two isoforms diverges at the 5′ end (indicated by the stripped and dotted boxes). The EPLIN-β unique sequences allow the extension of the ORF by 160 aa at the amino terminus of EPLIN-α. The positions of in frame stop codons upstream to the AUG start codons for two EPLIN isoforms and the termination codons are denoted. FIG. 1B depicts the deduced amino acid sequence of EPLIN-β. The ORF of EPLIN-α starts at aa position 161 of EPLIN-β. The aa sequences of two EPLIN isoforms are identical except for Arg344 of EPLIN-β which has been replaced by Pro184Gly185 in EPLIN-α. The 52 aa sequence of a LIM domain is underlined. FIG. 1C depicts the alignment of the EPLIN LIM domain sequence with the LIM domain of the mutant SREBP-2, KIAA0750, plant transcription factor SF3, and muscle LIM protein. The signature cysteine and histidine residues of LIM domain are indicated by bold lettering. Amino acid sequence identities (o) and similarities (underlined) are indicated.

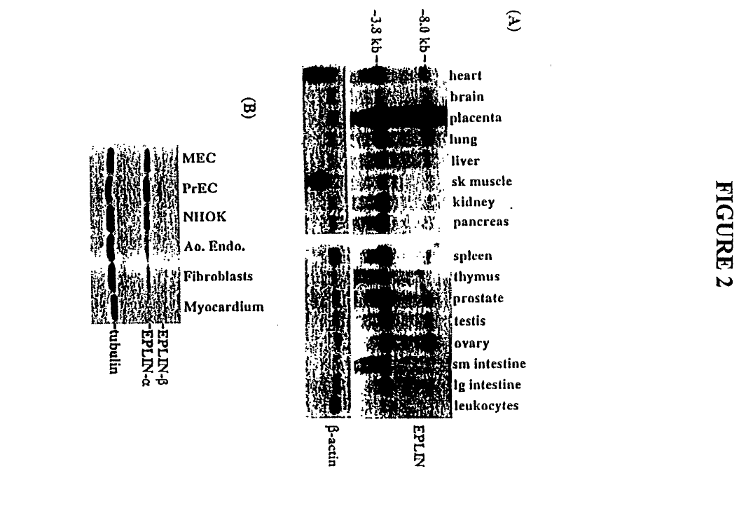

FIG. 2A depicts the distribution of EPLIN expression in different human adult tissues as determined by a Northern analysis. Filters containing mRNA from multiple human tissues (Clontech) were used for Northern blotting. The positions of ˜8 kb and ˜3.8 kb transcripts hybridized by the EPLIN probe are indicated (top). The same blot was re-probed with human b-actin cDNA (bottom).

FIG. 2B depicts the expression of EPLIN in different human primary cells were examined by an immunoblot analysis. MEC: mammary epithelial cells. PrEC: prostate epithelial cells. NHOK: normal human oral keratinocytes. Ao. Endo.: aortic endothelial cells. Fibroblasts: Dermal fibroblasts. Myocardium: human left ventricle. The positions of EPLIN-α and -β are noted. The loading of equivalent amounts of cell lysates was confirmed by probing the filter membrane with anti-a tubulin antibody.

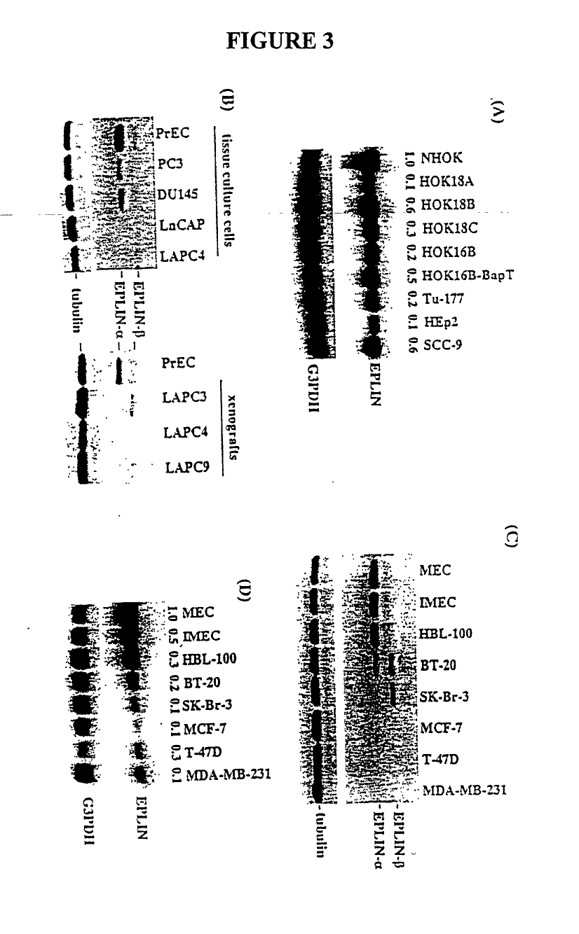

FIG. 3A depicts the expression of EPLIN transcripts in HPV-immortalized oral keratinocyte cell lines (HOK18A-C and HOK16B), tumorigenic HPV-transformed oral keratinocyte cell line (HOK16B-BapT), and oropharyngeal cancer cells (Tu-177, HEp2, and SCC-9) was determined by a Northern analysis (top). The filter membrane was re-probed with human G3PDH cDNA (bottom). The expression of EPLIN, normalized against the G3PDH, in each cell line is indicated. FIG. 3B depicts the expression of EPLIN proteins in different prostate cancer cell lines and xenograft tumors as determined by an immunoblot analysis. PrEC: prostate epithelial cells. PC3 and DU145: PSA-negative prostate cancer cell lines. LnCAP, LAPC3, LAPC4, and LAPC9: PSA-positive prostate cancer cells or xenograft tumors. The positions of EPLIN-α and -β are noted. The loading of equivalent amounts of cell lysates was confirmed by probing the filter membrane with anti-a tubulin antibody. FIG. 3C depicts the expression of EPLIN proteins in different breast cancer cell lines was determined by an immunoblot analysis. MEC: mammary epithelial cells. IMEC: immortalized mammary epithelial cells. HBL-100 is a non-tumorigenic breast cancer cells, while BT-20, SK-Br-3, MCF-7, T-47D, and MDA-MB-231 are tumorigenic breast cancer cell lines. The positions of EPLIN-α and -β are noted. The loading of equivalent amounts of cell lysates was confirmed by probing the filter membrane with anti-a tubulin antibody. FIG. 3D depicts the expression of EPLIN transcripts in different breast cancer cell lines was determined by a Northern analysis (top). The filter membrane was re-probed with human G3PDH cDNA (bottom). The expression of EPLIN, normalized against the G3PDH, in each cell line is indicated.

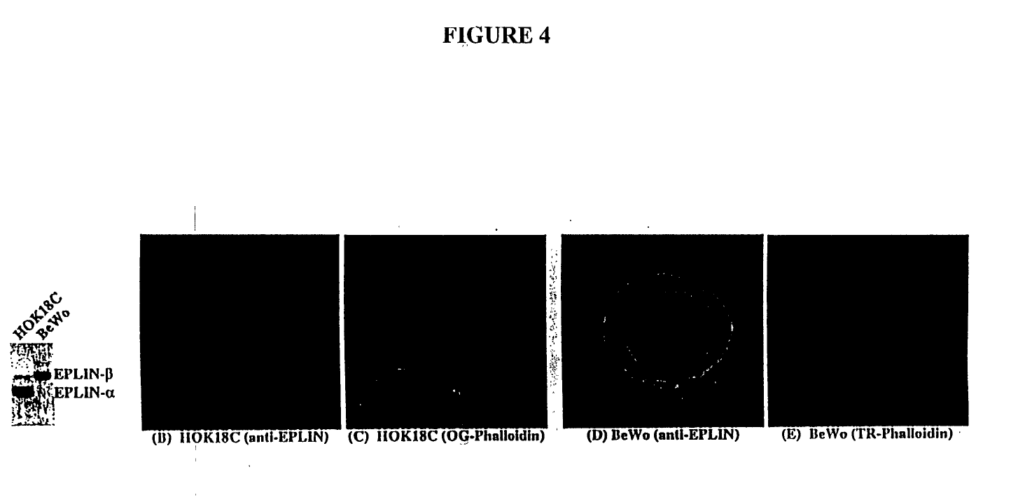

FIG. 4A depicts the relative amount of EPLIN isoforms in HOK18C (an HPV-immortalized human oral keratinocyte cell line) and BeWo (a human choriocarcinoma cell line) was determined by an immunoblot analysis. EPLIN-α is expressed as the major isoform in HOK18C, while EPLIN-β is the major isoform in BeWo. Figures B-E show the subcellular localization of EPLIN was determined by in situ immunofluorescence using anti-EPLIN antibodies (B and D). The staining pattern of anti-EPLIN antibodies (B and D) overlapped with the staining of actin stress fibers (C and E). Texas Red-conjugated goat anti-rabbit IgG secondary antibody (B) and fluorescein isothiocyanate-conjugated goat anti-rabbit IgG secondary antibody (D) were used to detect EPLIN. The stress fibers were stained with Oregon Green-phalloidin [symbol] and Texas Red-phalloidin (E).

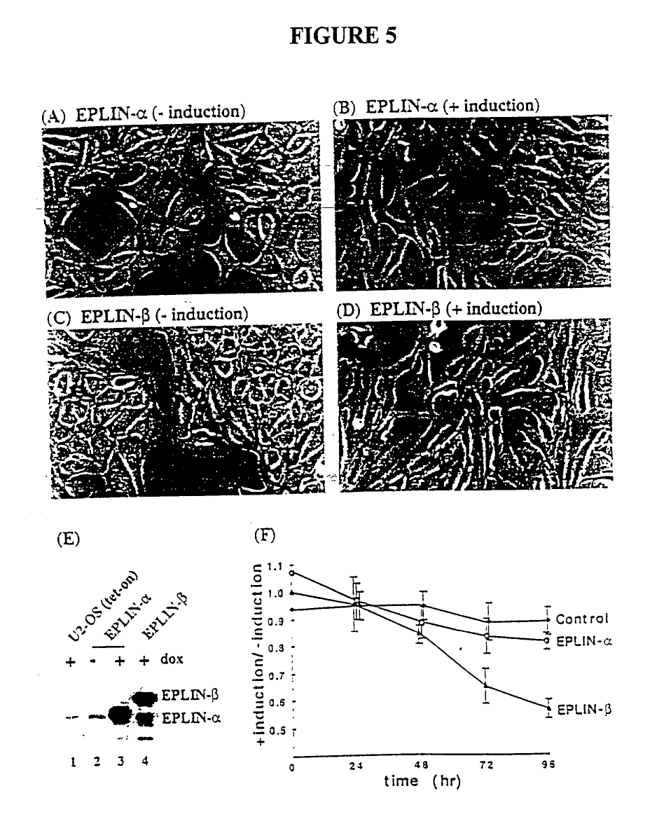

Figures A-D show the U2-OS osteosarcoma cells were engineered to express either EPLIN-α or -β isoform under the control of a tetracycline-inducible promoter (Tet-On). The appearance of U2-OS cells cultured with (B and D) and without (A and C) the induction of EPLIN are shown. Note that the expression of EPLIN changed the morphology of the U2-OS cells from round polygonal cells to fusiform cells characterized by asymmetric cytoplasmic extensions. FIG. 5E depicts the levels of EPLIN expression in the U2-OS cells cultures minus (no induction) and plus (induction) doxycycline were determined by an immunoblot analysis using anti-EPLIN antisera. Lane 1, parental U2-OS (Tet-on) cells; lane 2, U2-OS (EPLIN-α) cells before the induction; lane 3 and 4, U2-OS (EPLIN-α) and U2-OS (EPLIN-β) cells 48 h after the induction. FIG. 5F depicts the growth of U2-OS cells is presented as the ratio of cell numbers with and without EPLIN induction. For each time point, cell growth in triplicates were determined by a tetrazolium dye inclusion method.

cDNA fragments containing a partial open reading frame (ORF) were identified by the presence of a LIM domain. Clone #21 was used as a probe to isolate several cDNA clones from a HeLa cell cDNA library. Sequence analysis of these cDNA clones allowed for the assembly of an ORF of 600 aa (EPLIN-α) and an isoform (EPLIN-β) that extended an additional 160 aa at the amino terminus (FIG. 1A and 1B). The EPLIN-β mRNA also contained a deletion of 3 nucleotides within the coding region, introducing an Arg in place of ProGly at the corresponding position of EPLIN-α. Southern analysis indicated that EPLIN is a single copy gene, suggesting that the two EPLIN isoforms are generated by an alternative pre-RNA processing event. The predicted amino acid sequence of EPLIN was notable for a single centrally located LIM domain that is homologous to the partial ORF of a hamster gene of an unknown function. The EPLIN LIM domain is distantly related to the LIM domains of plant transcription factors SF-3 and the muscle LIM protein (FIG. 1C). Outside the LIM domain, EPLIN is unique in sequence, displaying no significant homology to known proteins or recognizable motifs.

Northern blot analysis of poly (a)+-RNA derived from normal human adult tissues demonstrated the expression of two EPLIN transcripts of ˜3.8 kb and ˜8 kb in size (FIG. 2A). The highest level of EPLIN mRNA was observed in placenta, followed by kidney, pancreas, prostate, ovary, spleen, and heart. A low level of EPLIN mRNA was also detected in all other tissues. The ORFs for both EPLIN-α and -β can be assembled from cDNA clones with insert sizes of ˜3.6 kb, corresponding the ˜3.8 kb transcript seen on the Northern blot.

Polyclonal anti-EPLIN antisera directed against the carboxy-terminal region common to both α and β isoforms was prepared. In normal primary mammary (MEC), prostate (PrEC), and oral (NHOK) epithelial cells, anti-EPLIN antisera detected a major protein band of 90 kD and a second minor species of 110 kD in molecular weight (FIG. 2B). These two species were assigned EPLIN-α and EPLIN-β, respectively.

A Northern analysis of immortalized or transformed oropharyngeal cell lines confirmed a consistent down-regulation of EPLIN transcripts to 10 to 60% of the level seen in the NHOK (FIG. 3A). An immunoblot analysis demonstrated a reduction in EPLIN protein in these cell lines. Using anti-EPLIN antisera, we extended the expression analysis of EPLIN to different types of human cancers. An immunoblot analysis using cell lysates prepared from 4 human prostate cancer cell lines showed significant changes of EPLIN expression (FIG. 3B). In two PSA-negative prostate cancer cell lines, PC3 and DU145, EPLIN expression was detectable, but at significantly reduced levels compared to the level seen in the normal primary prostate epithelial cells (PrEC). In two PSA-positive prostate cancer cell lines, LnCap and LAPC4, the expression of EPLIN-α was not detectable, while EPLIN-β continued to be expressed at a level comparable to that in the PrEC. Examination of human prostate tumors propagated in SCID mice also demonstrated the loss of EPLIN-α expression in LAPC3, LAPC4, and LAPC9 xenografts.

A survey of breast cancer cell lines revealed a similar change in EPLIN expression (FIG. 3C). Both immortalized mammary epithelial cells (IMEC) and HBL-100, a non-tumorigenic breast cancer cell line, expressed EPLIN-α and -β isoforms at levels equivalent to that seen in the MEC. In the BT-20 breast cancer cells, there was a reduction in EPLIN-α accompanied by an increase in EPLIN-β. A similar increase EPLIN-β expression was also seen in the SK-Br-3 breast cancer cells which lacked the EPLIN-α expression. In three other breast cancer cell lines, EPLIN-α was either absent (MCF-7 and T-47D) or significantly reduced (MDA-MB-231), while EPLIN-β continued to be expressed at a level equivalent to that in the MEC. A Northern analysis demonstrated a reduction in EPLIN transcripts in all of the breast cancer cell lines, confirming that the loss of EPLIN proteins is due to a transcriptional down-regulation (FIG. 3D).

EPLIN is a cytoskeletal protein that can alter cell morphology and suppress cell proliferation.

To investigate the potential function of EPLIN, the subcellular distribution of endogenous EPLIN was determined. Since the available polyclonal anti-EPLIN antisera do not distinguish the two known isoforms of EPLIN, in situ immunofluorescence was performed using two different cell lines, HOK18C (an immortalized human oral keratinocyte line), and BeWo (a human choriocarcinoma cell line). EPLIN-α is expressed as the predominant form in HOK18C, while EPLIN-β is the predominant form in BeWo (FIG. 4A). In situ immunofluorescence analysis demonstrated the localization of both EPLIN-α and -β to the cytoplasm in a fibrillar pattern at the periphery of the cell (FIG. 4B and D). This pattern of staining is similar to the staining of actin fibers with phalloidin (FIG. 4C and E). In addition, there was an overlap in the EPLIN staining to the paxillin staining (data not shown), suggesting that EPLIN is a component of the focal adhesion plaque.

To identify the effect of EPLIN on cell growth, each isoform was expressed in U2-OS osteosarcoma cells under the control of a tetracycline-inducible promoter. U2-OS cells, like most other cells, express EPLIN-α as the major isoform and a small amount of EPLIN-β isoform (FIG. 5E). Ectopic expression of either EPLIN isoform altered the morphology of the U2-OS cells from round polygonal cells with a cobblestone appearance to larger fusiform cells with spindle cell features and cytoplasmic extensions (FIG. 5A-D). In addition, the EPLIN overexpressing cells required a longer incubation time in trypsin for detachment, suggesting a change in the cell-matrix interaction. An analysis of cell proliferation using a tetrazolium dye inclusion assay revealed that the induction of EPLIN-β suppresses cell growth (FIG. 5F). While the effect was not as pronounced, a growth inhibition was also seen when EPLIN-α was overexpressed.

The present invention provides a novel gene, EPLIN, that is down-regulated in human cancer cells. Although the expression of EPLIN varied considerably in different adult tissues, there was a general tendency of higher expression in tissues rich in epithelial cells. This preferential expression of EPLIN in epithelial cells was substantiated by an immunoblot analysis demonstrating high levels of EPLIN expression in the normal epithelial cells (e.g., MEC, PrEC, NHOK). Low levels of EPLIN were also detected in the primary aortic endothelial cells and fibroblasts, but not in the myocardium. The absence of EPLIN proteins in the myocardium, in view of the relative abundance of EPLIN transcripts in the same tissue, suggest that the steady state level of EPLIN proteins can be subjected to a posttranslational regulation. The reduction in EPLIN protein in cancer cells in general paralleled the reduction in EPLIN transcripts (see FIG. 3C and D).

EPLIN sequence analysis revealed a single centrally located LIM domain. This motif may allow EPLIN to interact with other cellular proteins. Several LIM domain proteins have been implicated in cellular transformation. LMO-2 (formerly called RBTN2/TTG2), which interacts with the basic-helix-loop-helix protein Ta1/Sc1, is aberrantly expressed in acute T-cell leukemia as a result of chromosomal rearrangement and can promote T-cell tumors (Rabbitts, Genes and Devel., 12:2651, 1998). ril and DRAL are proteins of unknown function that are transcriptionally down-regulated in Ras-transformed cells and rhabdomyosarcoma cells, respectively (Kiess et al., Oncogene, 10:61, 1995; Genini et al., DNA and Cell Biol., 16:433, 1997). Many LIM domain proteins are involved in cell lineage determination as DNA-binding transcription factors or accessory factors that associate with DNA-binding transcription factors to modulate gene transcription (Dawid et al., Trends in Genetics 14:156, 1998). Other LIM domain proteins interact with cytoskeletal proteins or localize to the site of cell-matrix attachment. This class of LIM domain proteins includes zyxin (Beckerle, Bioessays, 19:949, 1997); paxillin, hic-5, and leupaxin (Brown et al., J. Cell Biol., 135:1109, 1996); LIM-kinase (Yang et al., J. Biol. Chem., 270:12152, 1998); and limatin (Roof et al., J. Cell Biol., 138:575, 1997). The amino acid sequence of the EPLIN LIM domain is closely related to the LIM domain of the plant transcription factor SF3 (48% aa identity; 73% aa similarity within the 52 aa LIM domain).

In situ immunofluorescence studies showed localization of both EPLIN-α and -β isoforms to filamentous actin-rich areas at the periphery of the cell. Furthermore, there was a frequent overlap between EPLIN staining and paxillin staining, suggesting that EPLIN maybe present in the focal adhesion plaques as well. The overexpression of EPLIN appears to affect the cell-matrix interactions as evidenced by changes in cell morphology. While the subcellular localization of endogenous EPLIN-α and -β isoforms was indistinguishable, ectopic expression of EPLIN-β had a more pronounced effect on the growth of U2-OS cells, suggesting a potential functional difference between the two EPLIN isoforms.

EPLIN expression is down-regulated in the majority of cancer cell lines examined in the present study, indicating that the loss of EPLIN expression is directly linked to cellular transformation. Breast and prostate cancer cells, but not the oropharyngeal cancer cells, exhibited a specific loss of EPLIN-α isoform. The loss of EPLIN-α isoform was accompanied by an increase in EPLIN-β isoform in 2/6 breast cancer cell lines (e.g., BT-20 and SK-Br-3). The combined levels of the two EPLIN isoforms were lower in BT-20 and SK-Br-3 cells.

The major difference between the two EPLIN isoforms is at the amino terminus where the b isoform contains an extension of 160 aa. The sequence divergence may be an alternative pre-mRNA splicing event involving a single pre-mRNA that utilizes alternative exons. Alternatively, EPLIN is transcribed from two distinct promoters to generate two pre-mRNA species both of which are spliced to the common 3′ exons. The relative increase in EPLIN-β in breast cancer cell lines BT-20 and Sk-Br-3, which have lost the expression of EPLIN-α, indicates that the expression of the two EPLIN isoforms can be regulated independently.

### EXAMPLE 2

### Isolation of Mouse and Zebrafish EPLIN cDNAs

EST clones 1885402 and 1922179, encoding mouse EPLIN-α and β, respectively, and EST clone 2352091, encoding zebrafish EPLIN, were purchased from Research Genetics (Huntsville, Ala.). EST clone 1885402 was sequenced in entirety, while only the regions corresponding to the open reading frames were sequenced in EST clones 1922179 and 2352091. The nucleotide sequences of mouse and zebrafish EPLIN have been deposited to Genbank with accession numbers as follows: AF307844 for mouse EPLIN-α; AF307845 for mouse EPLIN-β; and AF307846 for zebrafish EPLIN.

Analysis of Mouse EPLIN Gene Structure

High-density filters generated from the RPCI-22 129S6/SvEvTac Mouse BAC Library was purchased from BACPAC Resources (Oakland, Calif.) and screened using the 3′ coding region of mEPLIN-α (nt 1920-2243) as the probe. Of seven positive clones, clone RCPI22-121N19 was selected for further analysis. The human EPLIN gene structure information was used as a guide to predict the exon-intron boundaries of mouse EPLIN. The Bac clone RCPI22-121N19 was sequenced using primers derived from mouse EPLIN cDNA, near the predicted exon-intron boundaries, to obtain intronic sequences. FIG. 6 shows the amino acid sequence alignment of Mouse (m), human (h), and zebrafish (zf) EPLIN carried out using the Multiple Alignment Program. Sequence identity and gaps, relative to mEPLIN-b, are denoted by (.) and (−). Met162 of mEPLIN-β, corresponding to the initiation codon for mEPLIN-α is denoted by (*). Homology domains I to VII are boxed and homology domain IV includes the LIM domain of EPLIN.

EPLIN Expression in Mouse Tissue by Semi-quantitative RT-PCR

Normalized first-strand mouse cDNA (Clonetech, Palo Alto, Calif.) was subjected to two-step PCR according to manufacturer's suggestions in order to quantify the levels of mEPLIN-α and -β expression in different mouse tissues. One primer common to both mEPLIN-α and -β (5′-TCATGCCTGGAATCTCCCAGACAG-3′) (SEQ ID NO:39) was used in combination with primers specific to the 5′ end of either mEPLIN-α (5′-GACGGTGGACCAGGC ACAGCTTGG-3′) (SEQ ID NO:40) or mEPLIN-β (5′-GGTGTCTCAGAGTCTGTAGACAAG-3′) (SEQ ID NO:41) cDNAs. Products from a non-saturating number of cycles was subjected to gel electrophoresis, transferred to nylon membrane (Hybond, Amersham, Piscataway, N.J.) and hybridized with [³²P]-radiolabeled mouse EPLIN-α or -β specific DNA fragments. The hybridization signal was quantified on a phosphorimager (Molecular Dynamics, Sunnyvale, Calif.), using the ImageQuaNT software.

The results of the semi-quantitative RT-PCR are shown in FIG. 8. Amplified products of mEPLIN-α and -β mRNA were fractionated on a 1.5% agarose gel, transferred onto a nylon membrane, and then hybridized with [³²P]-radiolabeled probes specific for either mEPLIN-α or -β isoform (FIG. 8, Panel A). For PCR controls, mEPLIN cDNAs corresponding to the α and β isoforms were used. Negative (−) control denotes absence of added template. The hybridization signals were quantified and normalized against the signal from the tissue with the highest expression (7 day embryo for mEPLIN-α and kidney for mEPLIN-β ) (FIG. 8, Panel B).

EPLIN Expression in Mouse Tissue by Immunoblot

Mouse tissue was obtained from severe-combined immunodeficient (SCID) mice and immediately frozen on dry ice. Frozen tissue was finely chopped with a sterile blade and then boiled in a lysis buffer (25 mM Tris-HCl, pH 7.5, 1 mM EDTA, 0.2% SDS) for 5 min. Cleared tissue lysates were fractionated by SDS-PAGE under reducing conditions and transferred to nitrocellulose membrane. The membrane was immunoblotted with polyclonal anti-EPLIN antisera against the carboxy terminal region, common to both α and β isoforms of human EPLIN. The results of the immunoblot are shown in FIG. 8, Panel C.

Eukaryotic Expression and Subcellular Localization of EPLIN

EST 1922179 was used as the template to amplify both the mEPLIN-α and -β coding sequences using Pfu DNA polymerase (Strategene, La Jolla, Calif.). The amplified fragments were digested with BamH I and Sal I restriction enzymes and cloned into the corresponding sites of the pCMV5-FLAG expression vector to create N-terminal FLAG-epitope tagged mEPLIN-α and mEPLIN-β. Similarly, the zebrafish EPLIN coding region was amplified using EST 2352091 as the template and cloned into the pCMV5-FLAG expression vector.

Saos2 (human osteosarcoma) cells were cultured on glass coverslips coated with fibronectin in DMEM supplemented with 10% fetal calf serum and Pen/Strep. Exponentially growing cells were transfected with the EPLIN expression plasmids using Lipofectamine (Life Technologies). Twenty-four hours post-transfection, coverslips were transferred to individual wells of a 24-well tissue culture plate and fixed in 3.7% formaldehyde in PBS for 10 min and permeabilized in 0.2% Triton X-100 in PBS for 5 min. The slides were preincubated in a blocking buffer (0.1% Tween-20+10% goat serum in phosphate buffered saline). The flag-epitope tagged EPLIN was visualized by staining with the anti-FLAG M2 mAb (Sigma-Aldrich, St. Louis, Mo.) at 2 μg/ml for 30 min, followed by fluorescein isothiocyanate-conjugated goat anti-mouse IgG (Molecular Probes, Eugene, Oreg.). The F-actin was visualized using rhodamine-conjugated Phalloidin (Molecular Probes).

The results of the in situ immunofluorescence are shown in FIG. 9, Panels A-F. Human Saos2 osteosarcoma cells were transiently transfected with the mEPLIN-α (FIG. 9, Panels A and B), mEPLIN-β (FIG. 9, Panels C and D), and zfEPLIN (FIG. 9, Panels E and F) expression constructs. Twenty-four hours after the transfection, the cells were fixed and then stained for the transfected EPLIN (anti-Flag mAb) (FIG. 9, Panels A, C, and E) and the stress fibers (Rhodamine-Phalloidin) (FIG. 9, Panels B, D, and F).

### EXAMPLE 3

### Isolation of Distinct Promoters for EPLIN Isoforms

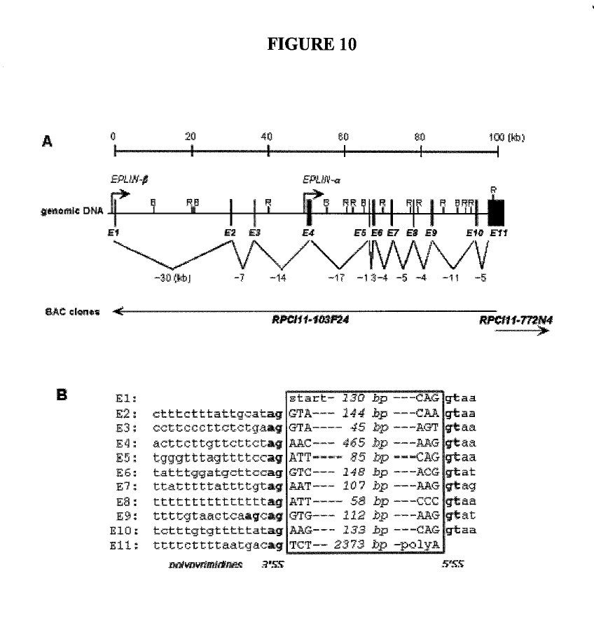

The National Center for Biotechnology Information Genome Survey Sequence database was searched with the human EPLIN cDNA sequence to identify BAC clones containing the human EPLIN gene. The Bac clone RPCI11-103F24 (Genbank accession AQ314676) that contains the bulk of the human EPLIN gene to the EcoR1 site at nt 2512 of the EPLIN-β cDNA was chosen for further analysis. The EPLIN cDNA corresponds to the unigene Hs.10706, which allowed us to establish the chromosome location to 12q13, between the region delimited by anchor markers D12S333 and D12S325. Bac DNA was digested with various restriction enzymes and subjected to Southern blot analysis using probes derived from various regions of EPLIN-α and -β cDNA to establish the overall genomic organization (FIG. 10, Panel A). Preliminary DNA sequence information of a 100 kb PAC clone mapping to the human chromosome 12q13-62.7-72 became available as unordered fragments (Genbank accession AC008147) which allowed for the design of primers to carry out long range PCR to determine intron sizes and generate exon-specific probes to refine the organization of EPLIN gene.

The coding sequence EPLIN is composed of 11 exons distributed over a >100 kb region. Exon 1 encodes the 5′ untranslated sequence and is followed by a large >30 kb intron. The ATG initiation codons for EPLIN-β and -α are located in Exons 2 and 4 respectively. The 54 aa LIM domain was encoded in Exons 9 and 10, split by an ˜11 kb intron. Both EPLIN isoforms share the same TGA stop codon located in Exon 11. This stop codon is followed by the 3′ untranslated sequence of ˜1.2 kb. The exon-intron boundary sequences are summarized in FIG. 1B. The sizes of introns 3, 5, 6, 7, and 10 were determined by long range PCR, while the sizes of remaining introns which failed to produce discrete fragments in long range PCR were estimated from the restriction mapping data. The 11 exons identified in this study contain all known cDNA sequences. Each internal exon is flanked by the conserved AG and GT splice sites. The 3′ splice site of Exon 9 contains two repeats of AG dinucleotides, both of which can be used as the splice sites. Exon 11 which is the 3′ most exon is exceptionally large and constitutes ˜⅔ of the coding sequences.

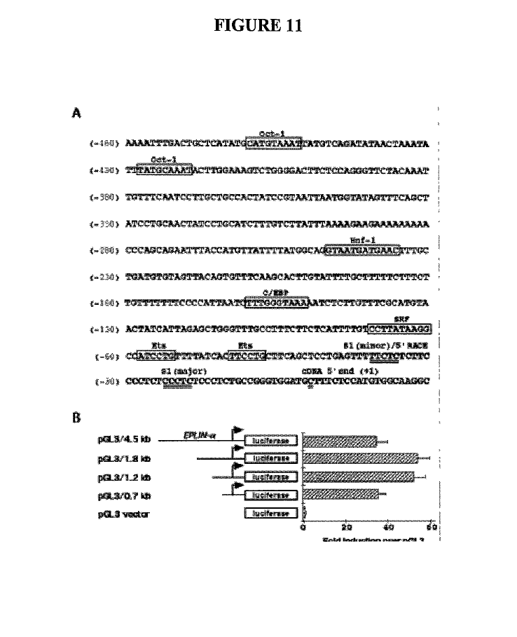

The 5′ end of EPLIN-α cDNA maps to intron 3, >50 kb downstream of the 5′ end of EPLIN-β in Exon 1. 5′ RACE was used to extend the 5′ end of EPLIN-α transcript by 37 nt (FIG. 11, Panel A). The additional sequence generated by the 5′ RACE was present in the genomic DNA, contiguous to the 5′ end of cDNA. A nuclease S1 protection also identified the 5′ ends of EPLIN-a transcript to nearby regions, indicating that the 5′ ends identified by 5′ RACE represent the transcriptional start sites for EPLIN-α.

The 5′ flanking sequences of EPLIN-α were cloned into the pGL3-Basic luciferase reporter vector to test for promoter activity. The constructs containing ˜4.5 kb, 1.8 kb, 1.2 kb, and 0.7 kb of the sequences upstream to the transcriptional start site gave luciferase activities ˜40 to ˜60-fold over the control promoterless pGL3-Basic vector in Hela cells (FIG. 11, Panel B). There were several potential transcription factor binding sites in the 5′ flanking region. One notable site was a serum response element (SRE) composed of a serum response factor (SRF) binding site (CCTTATAAGG) and neighboring Ets motifs (ATCCTG), located between −57 and −89.

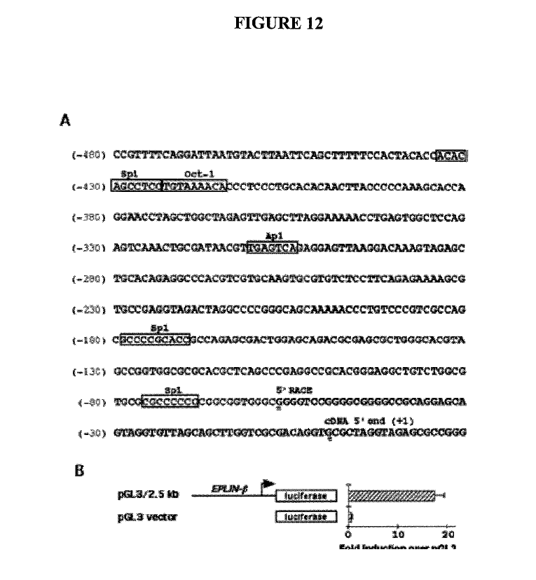

5′ RACE was employed in order to identify the transcriptional start site for EPLIN-β. The 5′ end of EPLIN-β transcripts was extended by 57 nt from the cDNA 5′ end (FIG. 12, Panel A). The additional sequences were contiguous with the genomic sequence, indicating that Exon 1 is the 5′ most exon of the EPLIN gene. The 5′ flanking sequences had a high GC content and contained several Sp1 consensus sites. In a promoter reporter assay, the 2.5 kb of the 5′ flanking sequences of EPLIN-β stimulated transcription by ˜20-fold over the promoterless pGL3-Basic vector in Hela cells, indicating that the 5′ end of EPLIN-β, identified by RACE, likely represents the transcriptional start site (FIG. 12, Panel B).

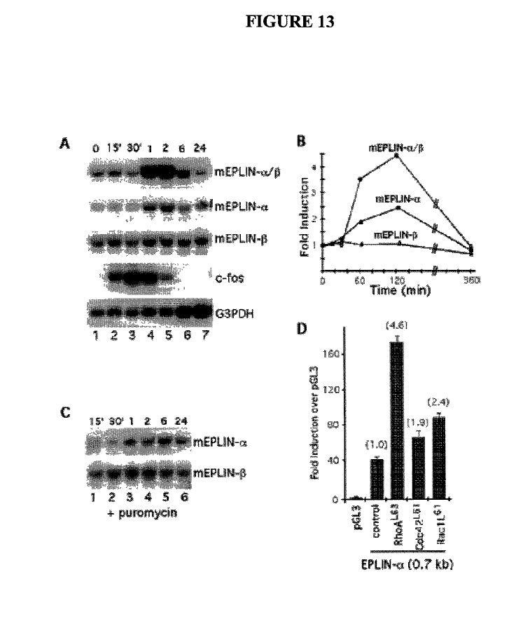

The expression of EPLIN-α is frequently down-regulated in breast and prostate cancer cells. The finding that the 5′ flanking sequences upstream of the transcriptional start sites of EPLIN-α and -β were capable of stimulating the expression of luciferase reporter indicated that two EPLIN isoforms were transcribed from two distinct promoters. The presence of a putative SRE in the EPLIN-α promoter indicates that the expression of EPLIN-α can be stimulated by serum. NIH3T3 cells were serum starved for 48 hrs and then stimulated with 15% newborn calf serum. A northern blot analysis using a probe derived from a region common to both EPLIN-α and -β revealed a specific transcript of ˜4 kb (FIG. 13, Panel A). The level of EPLIN transcripts increased over 4-fold following serum stimulation, peaking in 1 hr and staying elevated up to 2 hrs before returning to the baseline (FIG. 13, Panel B). Reanalysis of the northern blot using the probes specific for either EPLIN-α or -β demonstrated that the induction of EPLIN transcripts following serum stimulation is entirely due to the increase in the EPLIN-α transcript. The level of EPLIN-β remained constant during serum stimulation.

To determine whether the transcriptional response of EPLIN-α to serum is an immediate-early response, serum stimulation in the presence of puromycin to block protein synthesis was conducted. The serum stimulated induction of EPLIN-α was unaffected by this protein synthesis inhibitor, indicating the EPLIN-α is a primary response gene (FIG. 13, Panel C). The level of EPLIN-β mRNA was unaffected by serum stimulation or protein synthesis inhibitor.

The SRE consists of the binding site for ubiquitous SRF and an adjoining binding sites for ternary complex factors (TCF) of Ets family transcription, including Elk-1, Sap-1, and Sap-2/ERP/Net. The SRF can mediate transcriptional activation in response to serum, lysophosphatidic acids, or activation of Rho family small GTPases. A TCF is stimulated in response to activation of the Ras/Raf/MAP kinase pathway to form a ternary complex with SRF at the SRE. To test whether the consensus SRE found upstream of the EPLIN-α transcriptional sites is functional, activated forms of RhoA, Cdc42, and Rac1 with the 0.7 kb EPLIN-α promoter reporter construct were co-transfected (FIG. 13, Panel D). The activity of the 0.7 kb EPLIN-α promoter reporter construct could be stimulated 2 to 5 fold by these small GTPases, indicating that the consensus SRE found in the EPLIN-α promoter is functional.

The 5′ ends of EPLIN-α and -β are separated by >50 kb and the 5′ flanking regions of either EPLIN-α and -β are active in promoter reporter assays. The promoter region of EPLIN-β is notable for a high GC content. The promoter region contained several Sp1 binding sites that have been implicated in the basal transcription of many constitutively expressed house keeping genes. The promoter region of EPLIN-α contains several putative transcription factor binding sites including an SRE site which mediates the transcriptional response of many cellular genes to growth factor stimulation. EPLIN-α expression can be stimulated by serum in the absence of protein synthesis indicating that EPLIN regulates cell growth and differentiation. The known functions of EPLIN relate to cell morphology and cell proliferation further indicating that EPLIN regulates remodeling of actin stress fibers that occur following serum stimulation.

Analysis of Human EPLIN Gene Structure by Restriction Mapping

The Bac clone RPCI11-103F24 containing the bulk of human EPLIN gene 5′ to the EcoR1 site at the nucleotide (nt) position 2252 of EPLIN-β was purchased from Genome Systems. 0.25 mg Bac DNA was digested with Xba1, EcoRV, EcoR1, and BamH1, either singly or in combination, and used in Southern analysis. Each exon specific probes were generated by PCR from the Bac clone RPCI11-103F24 using the following primer sets:

| | | |
|---|---|---|
| Exon 1 | (CAGCAAAAACCCTGTCCCGTCGCC/CGACATAAGCACTCCCCACCCAGC); | (SEQ ID NOs:17 and 18) |
| Exon 2: | (TGTACCTAATCCTCACTCCTATTC/CAGTTTGGGGGAGAACAGTGGAGG); | (SEQ ID NOs:19 and 20) |
| Exon 3: | (ATACTTTAGGAGGCTGAAGCATGA/TGACATAGAACCCAAGCCCCACAG); | (SEQ ID NOs:21 and 22) |
| Exon 4: | (TTTCAATCCTTGCTGCCACTATCC/CTGACCACATATTCGCCTCTACTC); | (SEQ ID NOs:23 and 24) |
| Exon 5: | (GCACCTGTGAATGTGTAGTGCTTG/GCATTGGCTTCGAGTCCAAAAGAT); | (SEQ ID NOs:25 and 26) |
| Exon 6: | (TTGGCCAGTCACGGTGGTTTACAC/GACCAGGGCTCAGATACAAGGGTC); | (SEQ ID NOs:27 and 28) |
| Exon 7: | (GAGAGTTAGGGGGTTGATTTGACC/GCAAAAATGGCAATCTGTCCTGGC); | (SEQ ID NOs:29 and 30) |
| Exon 8: | (GCTGTTCTGGTGCCCGTTATTTCC/GCCATGAAGTGTGTAAAGGACATT); | (SEQ ID NOs:31 and 32) |
| Exon 9: | (GAGAATACCTGAGCCATAGTTGTT/TGCTCTTCTGTGGCATTATCATCC); | (SEQ ID NOs:33 and 34) |
| Exon 10: | (CATTTAGAGCAGCCACACAGTTTG/GCTCTAAAAGCCAAAGACTCCCTC); | (SEQ ID NOs:35 and 36) |
| Exon 11: | (GCTAAAATGGGGGTAAGGAATTCC/CCAAGATCAGCCCAACACTACCAT). | (SEQ ID NOs:37 and 38) |

Long Distance PCR

Long-distance PCR was utilized to determine the size of the introns. Using the Bac clone RPCI11-103F24 as the template, a primer pair flanking each intron was used to amplify the intron using Taq polymerase (Qiagen). The size of the amplified product was estimated by agarose-gel electrophoresis.

DNA Sequencing

The 5′ flanking regions of human EPLIN-α and -β were sequenced using the Bac clone RPCI11-103F24 as the template and primers derived from the 5′ regions of the published EPLIN sequences. The nucleotide sequence of the EPLIN promoters have been deposited to Genbank with accession numbers (INSERT) and (INSERT). To determine the sequence of exon-intron boundaries, each exon and the flanking sequences was PCR amplified using the primers described above and used as the template.

5′ RACE

RNA from HeLa cells, which express both EPLIN-α and -β, was used in 5′ RACE to identify putative transcriptional initiation sites. Briefly, 5 mg of total RNA was reverse transcribed using a primer specific for human EPLIN (5′-ATGGTCTGCTCTGTGCCTA ATCTC-3′) (SEQ ID NO:42) and SuperScriptII reverse transcriptase (Life Technologies). The cDNA was tailed with dCTP using terminal deoxynucleotidyl transferase and amplified by PCR using nested primers (EPLIN-α specific primer I: 5′—

| | | |
|---|---|---|
| (EPLIN-α specific primer I: | 5′-TAAGTAGATGCCAGTTATAACTTGC-3′; | (SEQ ID NO:43) |
| EPLIN-a specific primer II: | 5′-GTTAAAACTCCAAGCTGCTCC-3′; | (SEQ ID NO:44) |
| EPLIN-β specific primer I: | 5′-CCACAATAGCCGATGACTTGTTC-3′; | (SEQ ID NO:45) |
| EPLIN-β specific primer II: | 5′-TACAGACACTGAAATACCTGGC-3′) and the | (SEQ ID NO:46) |
| 5′ RACE abridged anchor primer | (5′-GGCCACGCGTCGACTAGTACGGGIIGGGIIGGGIIG-3′) | (SEQ ID NO:47) |

or the

| | | |
|---|---|---|
| Abridged Universal Amplification Primer | (5′-GGCCACGCGTCGACTAGTAC-3′). | (SEQ ID NO:48) |

The amplified products were purified using a 2% agarose gel electrophoresis
and subjected to DNA sequencing.

Construction of Promoter Reporter Plasmids

A 4.5 kb XbaI fragment containing the 5′ flanking region of EPLIN-a was first cloned into the XbaI site of pBSIIKS(+) to generate pBSIIKS(+)/4.5 kb EPLIN-α. The insert fragment was then excised from pBSIIKS(+)/4.5 kb EPLIN-α as a 4.5 kb SacI/XhoI fragment and cloned into the corresponding sites in pGL3-Basic (Promega). The 1.8 kb and 1.2 kb EPLIN-α promoter constructs were cloned by introducing the 1.8 kb HindIII or 1.2 kb BclI-HindIII fragment into the corresponding sites in pGL3-Basic. The 0.7 kb fragment used for the construction of pGL3/0.73 kb EPLIN-α was generated by PCR. EPLIN-β promoter reporter plasmid was constructed by cloning a 2.5 kb XbaI-XhoI fragment containing Exon 1 and the 5′ flanking region into the SacI-XhoI sites of pGL3-Basic.

Transfection Into Cells and Luciferase Assay

Hela cells were cultured in DMEM supplemented with 10% fetal bovine serum. One day prior to transfection, ˜400,000 cells were seeded on a 6 well plate. Approximately 16 fmole of each reporter construct and 5 ng of internal control pRL-TK (Promega) were transfected into cells using Lipofectamine reagent (Life Technologies). When indicated 100 ng of expression constructs for the activated forms of RhoA, Cdc42, and Rac1 were co-transfected with the 0.7 kb EPLIN-a promoter reporter construct. After 48 h, luciferease activity was determined using the Dual Luciferease Reporter Assay System (Promega). Each transfection was carried out in triplicate.

Serum Stimulation and Northern Blot Analysis

NIH3T3 cells were cultured in DMEM supplemented with 0.5% calf serum for 48 h. For serum stimulation, the culture medium was replaced with fresh medium containing 15% newborn calf serum. Serum stimulation in the absence of protein synthesis was carried out by supplementing the culture medium with puromycin (10 μg/ml). At indicated time points, RNA was prepared using RNA STAT-60 (Tel-Test). 5 μg of total RNA was used in northern analysis as previously described. Mouse EPLIN probes were prepared from EST clone 1885022 (EPLIN-α) and 1922179 (EPLIN-β). All probes were labeled with [³²P]-α-dCTP using a random prime labeling kit (Stratagene).

| | |
|---|---|
| Human EPLIN-α Nucleic Acid Sequence | |
| gctttctccatgtggcaaggctgtaactgttcacagctgtctgaaacagcagtggaccaggagcagcttggagt | (SEQ ID NO:1) |
| tttaactttcattttacaaagaacaacatgtttgaatgtttcagcaggcaagttataactggcatctacttctt | |
| gttcttctagaacaccgaaaatctctcccagcactttagaaaggggaccctgactgtgttaaagaagaagtggg | |
| agaacccagggctgggagcagagtctcacacagactctctacggaacagcagcactgagattaggcacagagca | |
| gaccatcctcctgctgaagtgacaagccacgctgcttctggagccaaagctgaccaagaagaacaaatccaccc | |
| cagatctagactcaggtcacctcctgaagccctcgttcagggtcgatatccccacatcaaggacggtgaggatc | |
| ttaaagaccactcaacagaaagtaaaaaaATGGAAAATTGTCTAGGAGAATCCAGGCATGAAGTAGAAAAATCA | |
| GAAATCAGTGAAAACACAGATGCTTCGGGCAAAATAGAGAAATATAATGTTCCGCTGAACAGGCTTAAGATGAT | |
| GTTTGAGAAAGGTGAACCAACTCAAACTAAGATTCTCCGGGCCCAAAGCCGAAGTGCAAGTGGAAGGAAGATCT | |
| CTGAAAACAGCTATTCTCTAGATGACCTGGAAATAGGCCCAGGTCAGTTGTCATCTTCTACATTTGACTCGGAG | |
| AAAAATGAGAGTAGACGAAATCTGGAACTTCCACGCCTCTCAGAAACCTCTATAAAGGATCGAATGGCCAAGTA | |
| CCAGGCAGCTGCGTCCAAACAAAGCAGCTCAACCAACTATACAAATGAGCTGAAAGCCAGTGGTGGCGAAATCA | |
| AAATTCATAAAATGGAGCAAAAGGAGAATGTGCCCCCAGGTCCTGAGGTCTGCATCACCCATCAGGAAGGGGAA | |
| AAGATTTCTGCAAATGAGAATAGCCTGGCAGTCCGTTCCACCCCTGCCGAAGATGACTCCCCAGGTGACTCCCA | |
| GGTTAAGAGTGAGGTTCAACAGCCTGTCCATCCCAAGCCACTAAGTCCAGATTCCAGAGCCTCCAGTCTTTCTG | |
| AAAGTTCTCCTCCCAAAGCAATGAAGAAGTTTCAGGCACCTGCAAGAGAGACCTGCGTGGAATGTCAGAAGACA | |
| GTCTATCCAATGGAGCGTCTCTTGGCCAACCAGCAGGTGTTTCACATCAGCTGCTTCCGTTGCTCCTATTGCAA | |
| CAACAAACTCAGTCTAGGAACATATGCATCTTTACATGGAAGAATCTATTGTAAGCCTCACTTCAATCAACTCT | |
| TTAAATCTAAGGGCAACTATGATGAAGGCTTTGGGCACAGACCACACAAGGATCTATGGGCAAGCAAAAATGAA | |
| AACGAAGAGATTTTGGAGAGACCAGCCCAGCTTGCAAATGCAAGGGAGACCCCTCACAGCCCAGGGGTAGAAAA | |
| TGCCCCTATTGCTAAGGTGGGTGTCCTGGCTGCAAGTATGGAAGCCAGGGCCTCCTCTCAGCAGGAGAAGGAAG | |
| ACAAGCCAGCTGAAACCAAGAAGCTGAGGATCGCCTGGCCACCCCCCACTGAACTTGGAAGTTCAGGAAGTGCC | |
| TTGGAGGAAGGGATCAAAATGTCAAAGCCCAAATGGCCTCCTGAAGACGAAATCAGCAAGCCCGAAGTTCCTGA | |
| GGATGTCGATCTAGATCTGAAGAAGCTAAGACGATCTTCTTCACTGAAGGAAAGAAGCCGCCCATTCACTGTAG | |
| CAGCTTCATTTCAAAGCACCTCTGTCAAGAGCCCAAAAACTGTGTCCCCACCTATCAGGAAAGGCTGGAGCATG | |
| TCAGAGCAGAATGAAGAATCTGTGGGTGGAAGAGTTGCAGAAAGGAAACAAGTGGAAAATGCCAAGGCTTCTAA | |
| GAAGAATGGGAATGTGGGAAAAACAACCTGGCAAAACAAAGAATTTAAAGGAGAGACAGGGAAGAGAAGTAAGG | |
| AAGGTCATAGTTTGGAGATGGAGAATGAGAATTTTGTAGAAAATGGTGCAGACTCCGATGAAGATGATAACAGC | |
| TTCCTCAAACAACAATTTCCACAAGAACCCAAGTTTTTGAATTGGTCGAGTTTTGTAGACAACACCTTTGCTGA | |
| AGAATTCACTACTCAGAATCAGAAATCCCAGGATGTGGAACTTTGGGAGGGAGAAGTGGTCAAAGAGCTCTCTG | |
| TGGAAGAACAGATAAAGAGAAATCGGTATTATGATGAGGATGAGGATGAAGAGTGAcaaattgcaatgatgctg | |
| ggccttaaattcatgttagtgttagcgagccactgccctttgtcaaaatgtgatgcacataagcaggtatccca | |
| gcatgaaatgtaatttacttggaagtaactttggaaaagaattccttcttaaaatcaaaaacaaaacaaaaaaa | |
| cacaaaaaacacattctaaatactagagataactttacttaaattcttcatcagtgatgatatgcataagtgct | |
| gtaaggcttgtaactggggaaatattccacctgataatagcccagattctactgtattcccaaaaggcaatatt | |
| aaggtagatagatgattagtagtatattgttacacactattttggaattagagaacatacagaaggaatttagg | |
| ggcttaaacattacgactgaatgcactttagtataaagggcacagtttgtatatttttaaatgaataccaattt | |
| aattttttagtatttacctgttaagagattatttagtctttaaattttttaggttaattttcttgctgtgatat | |
| atatgaggaatttactactttatgtcctgctctctaaactacatcctgaactcgacgtcctgaggtataacaac | |
| agagcactttttgaggcaattgaaaaaccaacctacactcttcggtgcttagagagatctgctgtctcccaaat | |
| aagcttttgtatctgccagtgaatttactgtactccaaatgattgctttcttttctggtgatatctgtgcttct | |
| cataattactgaaagctgcaatattttagtaataccttcgggatcactgtcccccatcttccgtgttagagcaa | |
| agtgaagagtttaaaggaggaagaagaaagaactgtcttacaccacttgagctcagacctctaaaccctgtatt | |
| tcccttatgatgtcccctttttgagacactaatttttaaatacttactagctctgaaatatattgatttttatc | |
| acagtattctcagggtgaaattaaaccaactataggcctttttcttgggatgattttctagtcttaaggtttgg | |
| ggacattataaacttgagtacatttgttgtacacagttgatattccaaattgtatggatgggagggagaggtgt | |
| cttaagctgtaggcttttctttgtactgcatttatagagatttagctttaatattttttagagatgtaaaacat | |
| tctgctttcttagtcttacctagtctgaaacatttttattcaataaagattttaattaaaatttg | |
| Human EPLIN-α Amino Acid Sequence | |
| MENCLGESRHEVEKSEISENTDASGKIEKYNVPLNRLKMMFEKGEPTQTKILRAQSRSASGRKISENSYSLDDLEIG | (SEQ ID NO:2) |
| PGQLSSSTFDSEKNESRRNLELPRLSETSIKDRMAKYQAAVSKQSSSTNYTNELKASGGEIKIHKMEQKENVPPGPE | |
| VCITHQEGEKISANENSLAVRSTPAEDDSPGDSQVKSEVQQPVHPKPLSPDSRASSLSESSPPKAMKKFQAPARETCVE | |
| CQKTVYPMERLLANQQVFHISCFRCSYCNNKLSLGTYASLHGRIYCKPHFNQLFKSKGNYDEGFGHRPHKDLWASKNENE | |
| EILERPAQLANARETPHSPGVEDAPIAKVGVLAASMEAKASSQQEKEDKPAETKKLRIAWPPPTELGSSGSALEEGIKM | |
| SKPKWPPEDEISKPEVPEDVDLDLKKLRRSSSLKERSRPFTVAASFQSTSVKSPKTVSPPIRKGWSMSEQSEESVGG | |
| RVAERKQVENAKASKKNGNVGKTTWQNKEsKGETGKRSKEGHSLEMENENlVENGADSDEDDNSFLKQQsPQEPKsLNWS | |
| SFVDNTFAEEFTTQNQKSQDVELWEGEVVKELSVEEQIKRNRYYDEDEDEE | |
| Human EPLIN-β Nucleic Acid Sequence | |
| ggcacgaggcgctaggtagagcgccgggacctgtgacagggctggtagcagcgcacaggaaaggcggcttttagcca | (SEQ ID NO:3) |
| ggtatttcagtgtctgtagacaagATGGAATCATCTCCATTTAATAGACGGCAATGGACCTCACTATCATTGAGGGT | |
| AACAGCCAAAGAACTTTCTCTTGTCAACAAGAACAAGTCATCGGCTATTGTGGAAATATTCTCCAAGTACCAGAAAG | |
| CAGCTGAAGAAACAAACATGGAGAAGAAGAGAAGTAACACCGAAAATCTCTCCCAGCACTTTAGAAAGGGGACCCTG | |
| ACTGTGTTAAAGAAGAAGTGGGAGAACCCAGGGCTGGGAGCAGAGTCTCACACAGACTCTCTACGGAACAGCAGCAC | |
| TGAGATTAGGCACAGAGCAGACCATCCTCCTGCTGAAGTGACAAGCCACGCTGCTTCTGGAGCCAAAGCTGACCAAG | |
| AAGAACAAATCCACCCCAGATCTACACTCAGGTCACCTCCTGAAGCCCTCGTTCAGGGTCGATATCCCCACATCAAG | |
| GACGGTGAGGATCTTAAAGACCACTCAACAGAAAGTAAAAAAATGGAAAATTGTCTAGGAGAATCCAGGCATGAAGT | |
| AGAAAAATCAGAAATCAGTGAAAACACAGATGCTTCGGGCAAAATAGAGAAATATAATGTTCCGCTGAACAGGCTTA | |
| AGATGATGTTTGAGAAAGGTGAACCAACTCAAACTAAGATTCTCCGGGCCCAAAGCCGAAGTGCAAGTGGAAGGAAG | |
| ATCTCTGAAAACAGCTATTCTCTAGATGACCTGGAAATAGGCCCAGGTCAGTTGTCATCTTCTACATTTGACTCGGA | |
| GAAAAATGAGAGTAGACCAAATCTGGAACTTCCACGCCTCTCAGAAACCTCTATAAAGGATCGAATGGCCAAGTACC | |
| AGGCAGCTGCGTCCAAACAAAGCAGCTCAACCAACTATACAAATGAGCTGAAAGCCAGTGGTGGCGAAATCAAAATT | |
| CATAAAATGGAGCAAAAGGAGAATGTGCCCCCAGGTCCTCAGGTCTGCATCACCCATCAGGAAGGGGAAAAGATTTC | |
| TGCAAATGAGAATAGCCTGGCAGTCCGTTCCACCCCTGCCGAAGATGACTCCCCAGGTGACTCCCAGGTTAAGAGTG | |
| AGGTTCAACAGCCTGTCCATCCCAAGCCACTAAGTCCAGATTCCAGAGCCTCCAGTCTTTCTGAAAGTTCTCCTCCC | |
| AAAGCAATGAAGAAGTTTCAGGCACCTGCAAGAGAGACCTGCGTGGAATGTCAGAAGACAGTCTATCCAATGGAGCG | |
| TCTCTTGGCCAACCAGCAGGTGTTTCACATCAGCTGCTTCCGTTGCTCCTATTGCAACAACAAACTCAGTCTAGGAA | |
| CATATGCATCTTTACATGGAAGAATCTATTGTAAGCCTCACTTCAATCAACTCTTTAAATCTAAGGGCAACTATGAT | |
| GAAGGCTTTGGGCACAGACCACACAAGGATCTATGGGCAAGCAAAAATGAAAACGAAGAGATTTTGGAGAGACCAGC | |
| CCAGCTTGCAAATGCAAGGGAGACCCCTCACAGCCCAGGGGTAGAAAATGCCCCTATTGCTAAGGTGGGTGTCCTGG | |
| CTGCAAGTATGGAAGCCAGGGCCTCCTCTCAGCAGGAGAAGGAAGACAAGCCAGCTGAAACCAAGAAGCTGAGGATC | |
| GCCTGGCCACCCCCCACTGAACTTGGAAGTTCAGGAAGTGCCTTGGAGGAAGGGATCAAAATGTCAAAGCCCAAATG | |
| GCCTCCTGAAGACGAAATCAGCAAGCCCGAAGTTCCTGAGGATGTCGATCTAGATCTGAAGAAGCTAAGACGATCTT | |
| CTTCACTGAAGGAAAGAAGCCGCCCATTCACTGTAGCAGCTTCATTTCAAAGCACCTCTGTCAAGAGCCCAAAAACT | |
| GTGTCCCCACCTATCAGGAAAGGCTGGAGCATGTCAGAGCAGAATGAAGAATCTGTGGGTGGAAGAGTTGCAGAAAG | |
| GAAACAAGTGGAAAATGCCAAGGCTTCTAAGAAGAATGGGAATGTGGGAAAAACAACCTGGCAAAACAAAGAATTTA | |
| AAGGAGAGACAGGGAAGAGAAGTAAGGAAGGTCATAGTTTGGAGATGGAGAATGAGAATTTTGTAGAAAATGGTGCA | |
| GACTCCGATGAAGATGATAACAGCTTCCTCAAACAACAATTTCCACAAGAACCCAAGTTTTTGAATTGGTCGAGTTT | |
| TGTAGACAACACCTTTGCTGAAGAATTCACTACTCAGAATCAGAAATCCCAGGATGTGGAACTTTGGGAGGGAGAAG | |
| TGGTCAAAGAGCTCTCTGTGGAAGAACAGATAAAGAGAAATCGGTATTATGATGAGGATGAGGATGAAGAGTGAcaa | |
| attgcaatgatgctgggccttaaattcatgttagtgttagcgagccactgccctttgtcaaaatgtgatgcacataa | |
| gcaggtatcccagcatgaaatgtaatttacttggaagtaactttggaaaagaattccttcttaaaatcaaaaacaaa | |
| acaaaaaaacacaaaaaacacattctaaatactagagataactttacttaaattcttcatcagtgatgatatgcata | |
| agtgctgtaaggcttgtaactggggaaatattccacctgataatagcccagattctactgtattcccaaaaggcaat | |
| attaaggtagatagatgattagtagtatattgttacacactattttggaattagagaacatacagaaggaatttagg | |
| ggcttaaacattacgactgaatgcacttagtataaagggcacagtttgtatatttttaaatgaataccaatttaatt | |
| ttttagtatttacctgttaagagattatttagtctttaaattttttaggttaattttcttgctgtgatatatatgag | |
| gaatttactactttatgtcctgctctctaaactacatcctgaactcgacgtcctgaggtataacaacagagcacttt | |
| ttgaggcaattgaaaaaccaacctacactcttcggtgcttagagagatctgctgtctcccaaataagcttttgtatc | |
| tgccagtgaatttactgtactccaaatgattgctttcttttctggtgatatctgtgcttctcataattactgaaagc | |
| tgcaatattttagtaataccttcgggatcactgtcccccatcttccgtgttagagcaaagtgaagagtttaaaggag | |
| gaagaagaaagaactgtcttacaccacttgagctcagacctctaaaccctgtatttcccttatgatgtccccttttt | |
| gagacactaatttttaaatacttactagctctgaaatatattgatttttatcacagtattctcagggtgaaattaaa | |
| ccaactataggcctttttcttgggatgattttctagtcttaaggtttggggacattataaacttgagtacatttgtt | |
| gtacacagttgatattccaaattgtatggatgggagggagaggtgtcttaagctgtaggcttttctttgtactgcat | |
| ttatagagatttagctttaatattttttagagatgtaaaacattctgctttcttagtcttacctagtctgaaacatt | |
| tttattcaataaagattttaattaaaatttg | |
| Human EPLIN-β Amino Acid Sequence | |
| MESSPFNRRQWTSLSLRVTAKELSLVNKNKSSAIVEIFSKYQKAAEETNMEKKRSNTENLSQHFRKGTLTVLKKKWEN | (SEQ ID NO:4) |
| PGLGAESHTDSLRNSSTEIRHRADHPPAEVTSHAASGAKADQEEQIHPRSRLRSPPEALVQGRYPHIKDGEDLKDHSTE | |
| SKKMENCLGESRHEVEKSEISENTDASGKIEKYNVPLNRLKMMFEKGEPTQTKILRAQSRSASGRKISENSYSLDDL | |
| EIGPGQLSSSTFDSEKNESRRNLELPRLSETSIKDRMAKYQAAVSKQSSSTNYTNELKASGGEIKIHKMEQKENVPP | |
| GPEVCITHQEGEKISANENSLAVRSTPAEDDSRDSQVKSEVQQPVHPKPLSPDSRASSLSESSPPKAMKKFQAPARE | |
| TCVECQKTVYPMERLLANQQVFHISCFRCSYCNNKLSLGTYASLHGRIYCKPHFNQLFKSKGNYDEGFGHRPHKDLWAS | |
| KNENEEILERPAQLANARETPHSPGVEDAPIAKVGVLAASMEAKASSQQEKEDKPAETKKLRIAWPPPTELGSSGSALEE | |
| GIKMSKPKWPPEDEISKPEVEPEDVDLDKKLRRSSSLKERSRPFTVAASFQSTSVKSPKTVSPPIRKGWSMSEQSEESV | |
| GGRVAERKQVENAKASKKNGNVGKTTWQNKEsKGETGKRSKEGHSLEMENENlVENGADSDEDDNSFLKQQsPQEPKsL | |
| NWSSFVDNTFAEEFTTQNQKSQDVELWEGEVVKELSVEEQIKRNRYYDEDEDEE | |
| Mouse EPLIN-α Nucleic Acid Sequence | |
| tgaacgacgg tggaccaggc acagcttgga gttttaactt tcagtttaca aagacgcatg 61 | (SEQ ID NO:9) |
| tttggctggg gcagcaggca agttgtaact ggtacttact tcttgttcct ctagaaccct 121 | |
| gaaagtctgc cccagcactt tagaagaggg accctgtctg tgttaaagaa gaagtgggag 181 | |
| aacccggtgg ctggggcaga attgcacaca gactcactgc caaacagcag cagtgagggt 241 | |
| gggcacacag cggactaccg tcctgctgaa gtgacggaca agcctgctcc tggagtcaga 301 | |
| gctgaccggg aagagcacac ccagcccaaa cctagatttg gatctcgtcc ggaagcagtt 361 | |
| atccagagcc ggtatcctcg ctcagagaac agccacgatt ttaaagccca ggccacggag 421 | |
| agccaaaaaa tggaaaactg tctgggagat tccaggcatg aagcagagaa gccagagacg 481 | |
| agcgaaaaca cagaaacttc aggcaaaata gagaaataca acgttccact gaatagactg 541 | |
| aagatgatgt ttgagaaagg tgaacacaac caaaccaaga gtctctggac ccaaagccga 601 | |
| aatgcgggtg gaaggaggct ctctgaaaac aactgttccc tggatgactg ggaaataggt 661 | |
| gcgggtcatt tgtcttcctc tgcattcaac tcggagaaga acgagagtaa gaggaatctg 721 | |
| gagctgccac gcctctcaga aacctccata aaggaccgca tggccaagta ccaggctgca 781 | |
| gtgtccaagc agagcagccc agccagctac acaaatgagc tgaaaaccag tgaaagcaaa 841 | |
| actcataaat gggagcagaa ggagaatgtg cctccaggtc ccgaggcctg cagcgtccat 901 | |
| caggaaggaa gcaaggtttc tgcaactgag aatagtctag tggccctctc tgtccctgct 961 | |
| gaagatgaca cctgtaactc ccaggtgaag agcgaggccc agcagcctat gcaccctaag 1021 | |
| ccgctgagtc ctgacgccag gacctccagt cttccggaaa gttctccttc caaaacagcg 1081 | |
| aagaagtttc aggcgccggc aaaggagagc tgcgtggagt gtcagaagac ggtgtacccc 1141 | |
| atggaacggc tcctggccaa ccagcaggtg tttcacatca gctgtttccg atgctcctac 1201 | |
| tgcaacaaca agctcagtct aggaacatat gcatccttac atggacgaat ctactgtaag 1261 | |
| cctcacttca atcaactctt caagtccaaa ggcaactatg acgagggctt tgggcacaag 1321 | |
| cagcataagg atctgtgggc aagcaagagc gacaatgagg agactttggg gagaccagcc 1381 | |
| cagccgccta atgcagggga gagctcccat agcccgggtg tagaagatgc ccccatcgcc 1441 | |
| aaggccggcg tgctggcggc aagtatggaa gccaaggcct cttctcagag ggagagggag 1501 | |
| gataagcctg cggagaccaa gaagctgagg atcgcctggc ctccgccagc tgagctgggc 1561 | |
| agttcgggaa gtgccctgga ggaagggatc aaagtatcga agcccaagtg gcctccggag 1621 | |
| gatgacgtct gcaagacgga ggccccggag gatgtagatc tggacctgaa gaagctgcgg 1681 | |
| cggtcttcgt ccctgaagga gaggagccgc ccgtttactg tagcagcttc ctttcgaacc 1741 | |
| tcttccatca agagccccaa gacctcttcg ccatctctca ggaaaggttg gagcgagtcc 1801 | |
| gagcagagtg aggagtttgg aggagggata gcgacgatgg aaaggaaaca aacggaaaat 1861 | |
| gccagaccct ctggggagaa agagaatgtg ggaaaatcac gctggcaggg cgaggaagtg 1921 | |
| ccgaggagca aggaccgtag ttcttttgag ctggagagtg agaattttat ggaaaatgga 1981 | |
| gcaaacatag ctgaagatga caaccatgtc cacgcacagc agtctccgtt agaacccgag 2041 | |
| gctccaggtt ggtctggctt tgtagacacc acggctgcta aagaattcac tacccagaat 2101 | |
| caaaaatccc aagatgtcgg gttctgggag ggagaagtgg tccgagagct gtctgtggag 2161 | |
| gaacagataa agagaaaccg gtactatgat gaggacgagg atgaagaatg accggccgcc 2221 | |
| atggtgctgg ccttagattt tggttagcaa ctcactgccc tttatcaagt ggtgcgcaga 2281 | |
| aacaggcagc ttggcgctaa gggtcattca tgtggaagca actttggaaa tgaattcctg 2341 | |
| ctagcaaaac agggcaaaaa caagcaaaaa caaaacagac aaacaaactc acccaaaacc 2401 | |
| aattctaagt gcattgtcaa aacaccggtg atcttttaag cctggtgtgg tggtaaacac 2461 | |
| ctttaatccc agcaccccac tcgggaggca gaggcagagg caggtgggtc tctctgagtt 2521 | |
| ccaggacagc cagggctaca aagagaaacc ttgcctcaaa acaaacaaac aaaaaatccc 2581 | |
| tccatttttg cagggaaggg ctaagcaggg atttctctca ctgatgctgc ctgtcatcca 2641 | |
| ctgcattccc caaatcaaga ttggtgtggt tagatattta gaggcttgtt ttcacactac 2701 | |
| agagaccatt gtggaattag agacacacac aacacattta ggagcccacc tcatgaccag 2761 | |
| aggcacttta gtataaaggg cacagtgtag ctattttacc aatttcattt tttagtactc 2821 | |
| aattgctgaa aatcaaatat ttagtctttc aagcttttga aatgcattct cttcctctgg 2881 | |
| tagataggga aagcttactg tctttcatcc tacatcctgt atttttaatc actgaggcac 2941 | |
| aaaacagcaa cctttggcca agtgaaaggc gctggttact gaagccggag cagcagtgtt 3001 | |
| caatccccaa aacctacgta aaggtgagag agcagactgc acagagctgt ccacacgtgc 3061 | |
| ccatcatcaa caataagaaa caagttaaaa aaaaaaatac aaccaacctc acaaacacct 3121 | |
| gcgagacaga gaaccaacct atatactttg gtgtttggag agacggacat tgtacccaga 3181 | |
| taatcagtgg cgaatgcagt gtgtctccct gcactctggg tgtttgccat ctcttctggt 3241 | |
| ggtttctcgc actttgcgga aagctgcagt gttgctgtaa gaccttttgg atcacgcttc 3301 | |
| accatgctcc aagtaaaaca acctgagatc agagctggga agcctcaggt cgaggcgaat 3361 | |
| cttggctctg aaatgtattg atttttaggc cagataattc ttccagggtt gcattaaatc 3421 | |
| acttttaggt ctgtcttctt aggccgtgtc ctactttgat tcattgtagt gataaaacac 3481 | |
| tgggcaggag caactcagag gggaagggtt tgtctccact cacaggccac gggtcacaca 3541 | |
| caggccacgc gcgcaccagc atggtcaagg gaagcgaggc cagaacccag aggcaagagc 3601 | |
| tgatgcagac cgtgtgggaa cactgcttgc tgacttcatc tctgtggctt actcaactgg 3661 | |
| ttttcttaca ccacccaggg ccacctggcc aggggtgaca ctgctcacaa caaaaacaga 3721 | |
| caaggcccac aggtgcatcg gttagaggcg gttcatcagc tgagtcccac atccaggtga 3781 | |
| ctctgtgtga ggctggtaaa cccagaggag gatagatggt tttctctggt attaaggctt 3841 | |
| tagaatatta taaacgagtg cagttcttgt atgtaactga tattccaaat gatgtgtatg 3901 | |
| gcggggtggg cttaaggtag tggcctcttt gcatttgtag agatttagct cagttttaga 3961 | |
| gatgtaaaat aatctgtttc cttatggcct tgtttag | |
| Mouse EPLIN-α Amino Acid Sequence | |
| MENCLGDSRHEAEKPETSENTETSGKIEKYNVPLNRLKMMFEKGEHNQTKSLWTQSRNAGGRRLSENNCSLDDW | (SEQ ID NO:10) |
| EIGAGHLSSSAFNSEKNESKRNLELPRLSETSIKDRMAKYQAAVSKQSSPASYTNELKTSESKTHKWEQKENVP | |
| PGPEACSVHQEGSKVSATENSLVALSVPAEDDTCNSQVKSEAQQPMHPKPLSPDARTSSLPESSPSKTAKKFQA | |
| PAKESCVECQKTVYPMERLLANQQVFHISCFRCSYCNNKLSLGTYASLHGRIYCKPHFNQLFKSKGNYDECFGH | |
| KQHKDLWASKSDNEETLGRPAQPPNAGESSHSPGVEDAPIAKAGVLAASMEAKASSQREREDKPAETKKLRIAW | |
| PPPAELGSSGSALEEGIKVSKPKWPPEDDVCKTEAPEDVDLDLKKLRRSSSLKERSRPFTVAASFRTSSIKSPK | |
| TSSPSLRKGWSESEQSEEFGGGIATMERKQTENARPSGEKENVGKSRWQGEEVPRSKDRSSFELESENFMENGA | |
| NIAEDDNHVHAQQSPLEPEAPGWSGFVDTTAAKEFTTQNQKSQDVGFWEGEVVRELSVEEQIKRNRYYDEDEDE | |
| E | |
| Mouse EPLIN-β Nucleic Acid Sequence | |
| gttgcgacag gtgcgctggg cagacagagc gcaggaccgt ggccgtgcag cgccgagccc 61 | (SEQ ID NO:11) |
| cgacggcttt ctagtcagcc cacaggtgtc tcagagtctg tagacaagat ggaatcgact 121 | |
| ccatttaata gacgccagtg gacttccctg tcattgagag taacagccaa agagctttct 181 | |
| cttgtcaaca agaacaagtc atccgcaatt gtggaaatat tctccaagta ccagaaggct 241 | |
| gctgaagaag ccaacatgga aaggaagaaa aataaccctg aaagtctgcc ccagcacttt 301 | |
| agaagaggga ccctgtctgt gttaaagaag aagtgggaga acccggtggc tggggcagaa 361 | |
| ttccacacag actcactgcc aaacagcagc agtgagggtg ggcacacagc ggactaccct 421 | |
| cctgctgaag tgacggacaa gcctgctcct ggagtcagag ctgaccggga agagcacacc 481 | |
| cagcccaaac ctagatttgg atctcgtccg gaagcagtta tccagagccg gtatcctcgc 541 | |
| tcagagaaca gccacgattt taaagcccag gccacggaga gccaaaaaat ggaaaactgt 601 | |
| ctgggagatt ccaggcatga agcagagaag ccagagacga gcgaaaacac agaaacttca 661 | |
| ggcaaaatag agaaatacaa cgttccactg aatagactga agatgatgtt tgagaaaggt 721 | |
| gaacacaacc aaaccaagag tctctggacc caaaaccgaa atgcgggtgg aaggaggctc 781 | |
| tctgaaaaca actgttccct ggatgactgg gaaataggtg cgggtcattt gtcttcctct 841 | |
| gcattcaact cggagaaaaa cgagagtaag aggaatctgg agctgccacg cctctcagaa 901 | |
| acctccataa aggaccgcat ggccaagtac caggctgcag tgtccaagca gagcagccca 961 | |
| gccagctaca caaatgagct gaaaaccagt gaaagcaaaa ctcataaatg ggaacagaag 1021 | |
| gagaatgtgc ccccaggtcc cgaggcctgc agcgtccatc aggaaggaag caaggtttct 1081 | |
| acaactgaga atagtctagt ggccctctct gtccctgctg aagatgacac ctgtaactcc 1141 | |
| caggtgaaga gcgaggccca gcagcctatg caccctaagc cgctgagtcc tgacgccagg 1201 | |
| acctccagtc ttccggaaag ttctccttcc aaaacagcga agaagtttca ggcgccggca 1261 | |
| aaggagagct gcgtggagtg tcagaagacg gtgtacccca tggaacggct cctggccaac 1321 | |
| cagcaggtgt ttcacatcag ctgtttccga tgctcctact gcaacaacaa gctcagtcta 1381 | |
| ggaacatatg catccttaca tggacgaatc tactgtaagc ctcacttcaa tcaactcttc 1441 | |
| aagtccaaag gcaactatga cgagggcttt gggcacaagc agcataagga tctgtgggca 1501 | |
| agcaagagcg acaatgagga gactttgggg agaccagccc agccgcctaa tgcaggggag 1561 | |
| agcccccata gcccgggtgt agaagatgcc cccatcgcca aggtcggcgt gctggcggca 1621 | |
| agtatggaag ccaaggcctc ttctcagagg gagagggagg ataagcccgc ggagaccaag 1681 | |
| aagctgagga tcgcctggcc tccgccagcc gagctgggcg gttccggaag tgccctggag 1741 | |
| gaagggatca aagtatcgaa gcccaagtgg cctccggagg atgacgtctg caagacggag 1801 | |
| gccccggagg atgtagatct ggacctgaag aagctgcggc ggtcttcgtc cctgaaggag 1861 | |
| aggagccgcc cgtttacggt agcagcttcc tttcgaacct cttccatcaa gagccccaag 1921 | |
| gcctcttcgc catctctcag gaaaggttgg agcgagtccg agcagagtga ggagtttgga 1981 | |
| ggagggatag cgacgatgga aaggaaacaa acggaaaatg ccagaccctc tggggagaaa 2041 | |
| gagaatgtgg gaaaatcacg ctggcagggc gaggaagtgc cgaggagcaa ggaccgtagt 2101 | |
| tcttttgagc tggagagtga gaattttatg gaaaatggag caaacatagc tgaagatgac 2161 | |
| aaccatgtcc acgcacagca gtccccgtta gagcccgagg ctccgggttg gtctggcttt 2221 | |
| gtagacacca cggctgctaa agaattcact acccagaatc aaaaatccca agatgtcggg 2281 | |
| ttctgggagg gagaagtggt ccgagagctg tctgtggagg aacagataaa gagaaaccgg 2341 | |
| tactatgatg aggacgagga tgaagaatga | |
| Mouse EPLIN-β Amino Acid Sequence | |
| MESTPFNRRQWTSLSLRVTAKELSLVNKNKSSAIVEIFSKYQKAAEEANMERKKNNPESLPQHFRRGTLSVLKK | (SEQ ID NO:12) |
| KWENPVAGAEFHTDSLPNSSSEGGHTADYPPAEVTDKPAPGVRADREEHTQPKPRFGSRPEAVIQSRYPRSENS | |
| HDFKAQATESQKMENCLGDSRHEAEKPETSENTETSGKIEKYNVPLNRLKMMFEKGEHNQTKSLWTQNRNAGGR | |
| RLSENNCSLDDWEIGAGHLSSSAFNSEKNESKRNLELPRLSETSIKDRMAKYQAAVSKQSSPASYTNELKTSES | |
| KTHKWEQKENVPPGPEACSVHQEGSKVSTTENSLVALSVPAEDDTCNSQVKSEAQQPMHPKPLSPDARTSSLPE | |
| SSPSKTAKKFQAPAKESCVECQKTVYPMERLLANQQVFHISCFRCSYCNNKLSLGTYASLHGRIYCKPHFNQLF | |
| KSKGNYDEGFGHKQHKDLWASKSDNEETLGRPAQPPNAGESPHSPGVEDAPIAKVGVLAASMEAKASSQRERED | |
| KPAETKKLRIAWPPPAELGGSGSALEEGIKVSKPKWPPEDDVCKTEAPEDVDLDLKKLRRSSSLKERSRPFTVA | |
| ASFRTSSIKSPKASSPSLRKGWSESEQSEEFGGGIATMERKQTENARPSGEKENVGKSRWQGEEVPRSKDRSSF | |
| ELESENFMENGANIAEDDNHVHAQQSPLEPEAPGWSGFVDTTAAKEFTTQNQKSQDVGFWEGEVVRELSVEEQI | |
| KRNRYYDEDEDEE | |
| Zebrafish EPLIN Nucleic Acid Sequence | |
| gtgagcggag cgttcagagc attaacagga cagggaagca cacaagaaca tccacttgaa 61 | (SEQ ID NO:13) |
| agactgtagg agggggcttg atcgactgca aactaaagaa tatccatccc tttgaagacg 121 | |
| tgaagaaagc aagatgtcag tgagttcatt tcggcgtggt caatgggcct cccagtcatt 181 | |
| gcgagtgacg gcaaaggagc tctccatagt cggtgtgcgg ggcaaaaaca cagccattgc 241 | |
| tgagcgcttt tccaagtacc agaaggcagc agaagaaaca agtttggaca agaaaaaatc 301 | |
| tccggagaaa tcgactccag gccttcgcaa cggcaatctg agtgtcctaa agcagctctg 361 | |
| ggaacatcca gccgaaacgc ctacatctcc agaacccaaa gcacacctcc agaaccatct 421 | |
| gcaacagtct gctgtaaaaa tcccactgga gtccactgac gtccagctta ttgagggcac 481 | |
| agaccagcag tgtttgagcg acagtgatca gccaatggag aagcgaacgc aaagagatgt 541 | |
| ggagaccctc atcgaaaagc ccaccgtccc cctcaacagc ctgaagatga tgttcgagaa 601 | |
| aggagagacc cttcatagta atgtgtccag agagcctggg acaacaggag acagtggatc 661 | |
| tgacaatatg gagccaggaa ctaaagaaag tctggagtgt ggggtaaaaa tgctggattc 721 | |
| aacccctctc agggacagga tggccatgta ccaggcagct gtgactaagc ttgatttccc 781 | |
| atcctccccc aatagtgaag cagcagacag tgaagcccga gctcacagtg gaaaacaaaa 841 | |
| ggaaaatgtg ccaccagtgt ctgctgatgt gtgccccgaa tccaacacta tcaaaagtcc 901 | |
| tgctccagac aggaatggtt ctgttttaag tcctgagcaa aaccagccta aattggttaa 961 | |
| gatgttccct ttgcctgtcc gtgaaacctg cgtgacgtgc cttaagactg tgtacccact 1021 | |
| agagaaactt gtagcgaatc agcagattta ccacaacacc tgtttccgct gtgcctactg 1081 | |
| caacactaaa ctcagtttgg tgaactacgc ctctttgcac aacaacgtct actgcaagcc 1141 | |
| acactactgc cagctgttta aagccaaggg caactacgat gagggtttcg gccaccgacc 1201 | |
| ccataaggag ctttgggaag gacgccccga aggagttgac gaccaagtaa agctttcacc 1261 | |
| tcaagaaacg agcctaacag tagaggagtc tccattggtc aaggtcaacg tgcttgcggc 1321 | |
| cactttagag acccggaccc aggccacgtc agaaagggtg gagaagccat tggaaacggg 1381 | |
| acgactcaag atctcctggc ctcctcagtc tgaaggggat gagagtgcaa cccacacagt 1441 | |
| aactgatggc agtggaatca aacccattcg ccctaaatgg cctccagagg gcgacactgt 1501 | |
| atcaagcaat gtggacctgg aatctgatct tccaaagctt cgcaggagcg tctcccttaa 1561 | |
| ggagagaagc aaaccgtttt gcatcttcga ctccgcaccc gtggctcaac ccaagagatg 1621 | |
| ccaaagatct ccgtcaaatg agaaaccaga ctccgaggag gaaatgtcac ccgtgtcttc 1681 | |
| caccacagac accaccattt cttctgagga catgactgaa cacaatcagt cggaggagga 1741 | |
| ggaccaggat aagaccaagg aggatgaaca gatggagcat gaggagaaag ttgatgtgca 1801 | |
| ggaggaggaa ctgtcttctc tgaaatgcag cacgccggat aacagccctc cattgtcacc 1861 | |
| tgagagcgag tctggattgg atcccgaaga gaaccaggct tcgcaagatg ttggattttg 1921 | |
| ggatggagaa gaagcggagg aagacaccgc tgatgtcact gtggaggact tgattaagag 1981 | |
| gaaccgtcat tatgatgatg aggatgatga agatgtggtc tgagtcatag attagatgga 2041 | |
| ctttaatttt ctcagaagca ccagcatact ttcatgtagt ctcttcattg tgggtctgtc 2101 | |
| attttcgtgc aattcttcat gttttcggct tttacatgtg cctataaatg ttactgaact 2161 | |
| gaaaaaaaaa caagtagttt tcaggttatt gggaaggttt ctctagtcta gatggtatgc 2221 | |
| tagagctctc gatttcttca tgtctagttt tatgaaattg tatttccttt ggtactattt 2281 | |
| gttttctata ttttctatat taatagacca ggatatttta ttctgctgct gttcgttttc 2341 | |
| tgaacacttg catttcgaaa tgattaaaaa agaaaatatg atggggccaa ttttaatgtt 2401 | |
| aagttcaaac acccatgttt tgttcgattg acactattat aatgcacttc aatgtacttt 2461 | |
| aaatgttaaa gcactctcct cgctgtacca cattgaattg gcaacctatg aatgttttag 2521 | |
| aattttccaa ttgtattatt taactgcatt ttaaatgtct tttttattat gggaccaaat 2581 | |
| accttgggaa aacttttgtt tcaaataaat ttacttctac aaaatcttaa g | |
| Zebrafish EPLIN Amino Acid Sequence | |
| MSVSSFRRGQWASQSLRVTAKELSIVGVRGKNTAIAERFSKYQKAAEETSLDKKKSPEKSTPGLRNGNLSVLKQ | (SEQ ID NO:14) |
| LWEHPAETPTSPEPKAHLQNHLQQSAVKIPLESTDVQLIEGTDQQCLSDSDQPMEKRTQRDVETLIEKPTVPLN | |
| SLKMMFEKGETLHSNVSREPGTTGDSGSDNMEPGTKESLECGVKMLDSTPLRDRMAMYQAAVTKLDFPSSPNSE | |
| AADSEARAHSGKQKENVPPVSADVCPESNTIKSPAPDRNGSVLSPEQNQPKLVKMFPLPVRETCVTCLKTVYPL | |
| EKLVANQQIYHNTCFRCAYCNTKLSLVNYASLHNNVYCKPHYCQLFKAKGNYDEGFGHRPHKELWEGRPEGVDD | |
| QVKLSPQETSLTVEESPLVKVNVLAATLETRTQATSERVEKPLETGRLKISWPPQSEGDESATHTVTDGSGIKP | |
| IRPKWPPEGDTVSSNVDLESDLPKLRRSVSLKERSKPFCIFDSAPVAQPKRCQRSPSNEKPDSEEEMSPVSSTT | |
| DTTISSEDMTEHNQSEEEDQDKTKEDEQMEHEEKVDVQEEELSSLKCSTPDNSPPLSPESESGLDPEENQASQD | |
| VGFWDGEEAEEDTADVTVEDLIKRNRHYDDEDDEDVV | |
| Human EPLIN-α protein (EPLIN) gene promoter region (GenBank Acc. No. AF245392). | |
| tttcaagagt atattcagaa gaagaatagg gtgagagact ttaattccca aaatagctgc 61 | (SEQ ID NO:15) |
| taatcctgtt taattcctag gttgttgtcc tttctttctt agtgaaagac tggtgagtca 121 | |
| cagactacag gagagaaggt attggagttt gcagtttgtt gggaaggtgg tgctgttcaa 181 | |
| tttatttagg agtacagtcc tttcaaagta cgggacattt aatttaattt aatttttctt 241 | |
| tttttttttg agacagagtc tcactctgtc acccggactg gagtgcagtg gcgcgatttg 301 | |
| ggctcactgc aacctccgcc tcccaggttc aagccatttt tctgcctcag cctcctgagt 361 | |
| agctgggact acaggcgcgt gccaccacgc ccagctattt tttgtatttt tagtagagac 421 | |
| gtggtttcac ggtgttggcc gggctggcca cgaactcctg acctcaggtg atccacccgc 481 | |
| ctcggcctcc caaagtgctg ggattacagg catgagccac tgcgcccagc cgggacattt 541 | |
| aattttcaac aagcgtgttt cctctattaa ggaacaacag tgacaatttc caggttgttt 601 | |
| tcagtctgtc ttacatgtat aaaataattc catttttggc cctagtgaaa agtgaactct 661 | |
| cttagatatg taaatgtgtg tcctttgatc atctgagtga taagtagtgg tatttgcatt 721 | |
| tgaagcctgc atattttaca accagaggat ctgtaaaaca gtgaatttct tgataactga 781 | |
| gaattttttt tttttttttt ggttggggga tggcgtctca ctttgtcacc caggctggag 841 | |
| tgtagtggcg cagtcttggc tcactgcaac ctccacttcc caggttcaag cgattctcct 901 | |
| gcctcagcct ccagagtagc tgggactatg ggtgtgcacc actgcaccca gttaattttt 961 | |
| gtattttttt tagagacagg gtttctccat gttggtctgg ctggtctcaa actcctgacc 1021 | |
| ttgtgatccg ccggcctcgg cctcccaaag tgctgggatt acaggcgtga gccaccgtgc 1081 | |
| ctggccctga taagtgagaa tttatacaca gataaacttg tagagaagag cagggaaaaa 1141 | |
| aaaaagatta aattcttatg aaaaataaaa tttgactgct catatgcatg taaattatgt 1201 | |
| cagatataac taaatattta tgcaaatact tggaaagtct ggggacttct ccagggttct 1261 | |
| acaaattgtt tcaatccttg ctgccactat ccgtaattaa tggtatagtt tcagctatcc 1321 | |
| tgcaactatc ctgcatcttt gtcttattta aaagaagaaa aaaaaaccca gcagaattta 1381 | |
| ccatgttatt ttatggcagg taatgatgaa ctttgctgat gtgtagttac agtgtttcaa 1441 | |
| gcacttgtat tttgcttttt ctttcttgtt tttttcccca ttaatctttg ggtaaaaatc 1501 | |
| tcttgtttcg catgtaacta tcattagagc tgggtttgcc tttcttctca ttttgtcctt 1561 | |
| ataaggccat cctgttttat cacttcctgc ttcagctcct gagtttttct ctcttcccct 1621 | |
| ctccctctcc ctctgccggg tggatgcttt ctccatgtgg caaggctgta actgttcaca 1681 | |
| gctgtctgaa acagcagtgg accaggagca gcttggagtt ttaactttca ttttacaaag 1741 | |
| aacaacatgt ttgaatgttt cagcaggcaa gttataactg gcatctactt cttgttcttc 1801 | |
| tagaacaccg | |
| Human EPLIN-β protein (EPLIN) gene promoter region (GenBank Acc. No. AF24539 1). | |
| tggcatattc atcacctgtt ggaaatctct cttctcacac attttcatta acttcttgag 61 | (SEQ ID NO:16) |
| gaagtgtaat tacaagcgtt ttcccacgag cagcccctac taataacgca tcaagctgca 121 | |
| tgaattccga aaagcttcag aaaacttgtg gtctgaaacc ctactatgct tgaggtacag 181 | |
| gaaagaagga tactatcaaa aggcatcatg cagctggcac ggaactggga caagaatttg 241 | |
| ggggcaggat ggccaagtgc taggccaatt gacggtctcc aaaccattag cacagctcct 301 | |
| attctgaatg gaggagtaaa aacagctgtt ggagaacttg gacgtcatct gcccttgtca 361 | |
| aaccgttttc aggattaatg tacttaattc agctttttcc actacaccac acagcctcct 421 | |
| gtaaaacacc ctccctgcac acaacttacc cccaaagcac caggaaccta gctggctaga 481 | |
| gttgagctta ggaaaaacct gagtggctcc agagtcaaac tgcgataacg ttgagtcaga 541 | |
| ggagttaagg acaaagtaga gctgcacaga ggcccacgtc gtgcaagtgc gtgtctcctt 601 | |
| cagagaaagg cgtgccgagg tagactaggc cccgggcagc aaaaaccctg tcccgtcgcc 661 | |
| agcgcccgca ccgccagagc gactggagca gacgcgagcg ctgggcacgt agccggtggc 721 | |
| gcgcacgctc agcccgaggc cgcacgggag gctgtctggc gtgcgcgccc ccgcggcggt 781 | |
| gggcggggtc cggggcgggg ccgcaggagc agtaggtgtt agcagcttgg tcgcgacagg 841 | |
| tgcgctaggt agagcgccgg gacctgtgac agggctggta gcagcgcaga ggaaaggcgg 901 | |
| cttttagcca ggtaagggcc ttctctctcc acaaccggag agtgcgggaa gaccccggct 961 | |
| tctctccaca ccctctttct ccttcgactt ttggggcggg gggagtgcaa ggagtgtgag 1021 | |
| tccctccccc tcgagataag gggtcgaggg acgctgatcc tccgttcttt cctcacttct 1081 | |
| ggggcctgcg ctgggtgggg agtgcttat | |

It will be apparent to those skilled in the art that various modifications and variations can be made to the compositions and processes of this invention. Thus, it is intended that the present invention cover such modifications and variations, provided they come within the scope of the appended claims and their equivalents.

What is claimed is:

1. A nucleic acid molecule encoding a vertebrate EPLIN protein.

2. The nucleic acid molecule of claim 1, wherein the molecule is a cDNA.

3. The nucleic acid molecule of claim 1, wherein the vertebrate is a mammal.

4. An isolated nucleic acid molecule selected from the group consisting of:
a) a nucleic acid molecule comprising the nucleotide sequence set forth in SEQ ID NO:1;
b) a nucleic acid molecule comprising the nucleotide sequence set forth in SEQ ID NO:3;
c) a nucleic acid molecule comprising the nucleotide sequence set forth in SEQ ID NO:9;
d) a nucleic acid molecule comprising the nucleotide sequence set forth in SEQ ID NO:11; and
e) a nucleic acid molecule comprising the nucleotide sequence set forth in SEQ ID NO:13.

5. An isolated nucleic acid molecule selected from the group consisting of:
a) a nucleic acid molecule which encodes a polypeptide comprising the amino acid sequence set forth in SEQ ID NO:2;
b) a nucleic acid molecule which encodes a polypeptide comprising the amino acid sequence set forth in SEQ ID NO:4;
c) a nucleic acid molecule which encodes a polypeptide comprising the amino acid sequence set forth in SEQ ID NO:10;
d) a nucleic acid molecule which encodes a polypeptide comprising the amino acid sequence set forth in SEQ ID NO:12; and
e) a nucleic acid molecule which encodes a polypeptide comprising the amino acid sequence set forth in SEQ ID NO:14.

6. An isolated nucleic acid molecule selected from the group consisting of:
a) a nucleic acid molecule which encodes a naturally occurring ortholog, homolog or allelic variant of a polypeptide comprising the amino acid sequence of SEQ ID NO:2, wherein the nucleic acid molecule hybridizes to a nucleic acid molecule comprising SEQ ID NO:1 under stringent conditions;
b) a nucleic acid molecule which encodes a naturally occurring ortholog, homolog or allelic variant of a polypeptide comprising the amino acid sequence of SEQ ID NO:4, wherein the nucleic acid molecule hybridizes to a nucleic acid molecule comprising SEQ ID NO:3 under stringent conditions;
c) a nucleic acid molecule which encodes a naturally occurring ortholog, homolog or allelic variant of a polypeptide comprising the amino acid sequence of SEQ ID NO:10, wherein the nucleic acid molecule hybridizes to a nucleic acid molecule comprising SEQ ID NO:9 under stringent conditions;
d) a nucleic acid molecule which encodes a naturally occurring ortholog, homolog or allelic variant of a polypeptide comprising the amino acid sequence of SEQ ID NO:12, wherein the nucleic acid molecule hybridizes to a nucleic acid molecule comprising SEQ ID NO:11 under stringent conditions; and
e) a nucleic acid molecule which encodes a naturally occurring ortholog, homolog or allelic variant of a polypeptide comprising the amino acid sequence of SEQ ID NO:14, wherein the nucleic acid molecule hybridizes to a nucleic acid molecule comprising SEQ ID NO:13 under stringent conditions.

7. An isolated nucleic acid molecule selected from the group consisting of:
a) a nucleic acid molecule comprising a nucleotide sequence which is at least 60% homologous to the nucleotide sequence of SEQ ID NO:1, 3, 9, 11, or 13, or a complement thereof;
b) a nucleic acid molecule comprising a fragment of at least 200 nucleotides of a nucleic acid comprising the nucleotide sequence of SEQ ID NO:1, 3, 9, 11, or 13, or a complement thereof;
c) a nucleic acid molecule which encodes a polypeptide comprising an amino acid sequence at least about 60% homologous to the amino acid sequence of SEQ ID NO:2, 4, 10, 12, or 14; and
d) a nucleic acid molecule which encodes a fragment of a polypeptide comprising the amino acid sequence of SEQ ID NO:2, 4, 10, 12, or 14, wherein the fragment comprises at least 15 contiguous amino acid residues of the amino acid sequence of SEQ ID NO:2, 4, 10, 12, or 14.

8. An isolated nucleic acid molecule that hybridizes to the nucleic acid molecule of any one of claims 4, 5, 6 or 7 under stringent conditions.

9. An isolated nucleic acid molecule comprising a nucleotide sequence that is complementary to the nucleotide sequence of the nucleic acid molecule of any one of claims 4, 5, 6 or 7.

10. An isolated nucleic acid molecule comprising the nucleic acid molecule of any one of claims 4, 5, 6, or 7, and a nucleotide sequence encoding a heterologous polypeptide.

11. An isolated nucleic acid molecule comprising a sequence that hybridizes under stringent conditions to a hybridization probe having the nucleotide sequence of the coding sequence of SEQ ID NO:1, 3, 9, 11 or 13, or of the complement of said sequence.

12. An isolated nucleic acid molecule selected from the group consisting of:
(a) SEQ ID NO:1, 3, 9, 11 or 13;
(b) SEQ ID NO:1, 3, 9, 11 or 13, wherein T can also be U;
(c) nucleic sequences complementary to SEQ ID NO:1, 3, 9, 11 or 13; and
(d) fragments of (a), (b), or (c) that are at least 15 bases in length and that will hybridize to DNA which encodes a polypeptide of SEQ ID NO:2, 4, 10, 12 or 14.

13. A vector comprising the nucleic acid molecule of any one of claims 1, 4, 5, 7, 11 or 12.

14. The vector of claim 13, which is an expression vector.

15. The vector of claim 14, wherein the vector is a viral vector.

16. A host cell transformed with a vector of claim 13.

17. The host cell of claim 16, wherein the cell is a eukaryotic cell.

18. The host cell of claim 16, wherein the cell is a prokaryotic cell.

19. An isolated polypeptide selected from the group consisting of:
a) a fragment of a polypeptide comprising the amino acid sequence of SEQ ID NO:2, 4, 10, 12, or 14, wherein the fragment comprises at least 15 contiguous amino acids of SEQ ID NO:2, 4, 10, 12, or 14;
b) a naturally occurring ortholog, homolog or allelic variant of a polypeptide comprising the amino acid sequence of SEQ ID NO:2, 4, 10, 12, or 14, wherein the polypeptide is encoded by a nucleic acid molecule which hybridizes to a nucleic acid molecule comprising SEQ ID NO:1, 3, 9, 11 or 13, under stringent conditions;
c) a polypeptide which is encoded by a nucleic acid molecule comprising a nucleotide sequence which is at least 60% homologous to a nucleic acid comprising the nucleotide sequence of SEQ ID NO:1, 3, 9, 11 or 13;
d) a polypeptide comprising an amino acid sequence which is at least 60% homologous to the amino acid sequence of SEQ ID NO:2, 4, 10, 12, or 14.

20. The isolated polypeptide of claim 19 comprising the amino acid sequence of SEQ ID NO:2, 4, 10, 12, or 14.

21. The polypeptide of claim 19, further comprising heterologous amino acid sequences.

22. A substantially purified EPLIN polypeptide comprising an amino acid sequence as set forth in SEQ ID NO:2 or SEQ ID NO:4.

23. A substantially purified polypeptide comprising an amino acid sequence encoded by a nucleic acid sequence which hybridizes to the nucleic acid sequence set forth in SEQ ID NO:1 or SEQ ID NO:3 under stringent conditions.

24. An isolated polypeptide selected from the group consisting of:
a) a polypeptide comprising an amino acid sequence which is at least 70% identical to the amino acid sequence set forth in SEQ ID NO:2;
b) an enzyme which comprises at least 30 amino acid residues to the enzyme of a); and
c) the amino acid sequence as set forth in SEQ ID NO:2.

25. A method of producing EPLIN polypeptide comprising:
a) transforming a host cell with a nucleic acid molecule of claim 1;
b) expressing the nucleic acid molecule in the host; and
c) recovering the EPLIN polypeptide.

26. An antibody that binds to a polypeptide of claim 19.

27. The antibody of claim 26, wherein the antibody is polyclonal.

28. The antibody of claim 26, wherein the antibody is monoclonal.

29. A method for identifying a compound which binds to EPLIN polypeptide comprising:
a) incubating components comprising the compound and EPLIN polypeptide under conditions sufficient to allow the components to interact; and
b) measuring the binding of the compound to EPLIN polypeptide.

30. The method of claim 29, wherein the compound is a peptide.

31. The method of claim 29, wherein the compound is a peptidomimetic.

32. The method of claim 29, wherein the compound is an antibody.

33. A method of detecting a neoplastic cell in a sample comprising: contacting a sample suspected of containing a neoplastic cell with a reagent that binds to an EPLIN-specific cell component; and detecting binding of the reagent to the component.

34. The method of claim 33, wherein the neoplastic cell is an epithelial cell.

35. The method of claim 34, wherein the EPLIN-specific component is nucleic acid which encodes EPLIN polypeptide, or fragments thereof.

36. The method of claim 35, wherein the nucleic acid is DNA.

37. The method of claim 35, wherein the nucleic acid is RNA.

38. The method of claim 33, wherein the EPLIN-specific cell component is the EPLIN polypeptide, or fragments thereof.

39. The method of claim 33, wherein the reagent is a probe.

40. The method of claim 39, wherein the probe is nucleic acid.

41. The method of claim 39, wherein the probe is an antibody.

42. The method of claim 41, wherein the antibody is polyclonal.

43. The method of claim 41, wherein the antibody is monoclonal.

44. The method of claim 39, wherein the probe is detectably labeled.

45. The method of claim 44, wherein the label is selected from the group consisting of a radioisotope, a bioluminescent compound, a chemiluminescent compound, a fluorescent compound, a metal chelate, or an enzyme.

46. The method of claim 33, wherein the sample is obtained from a subject selected from the group consisting of human, swine, porcine, feline, canine, equine, murine, cervine, caprine, lupine, leporidine and bovine.

47. A method of detecting a cell proliferative disorder in a sample from a subject, comprising:
a) contacting a first sample having, or suspected of having, a cell proliferative disorder with a reagent that binds to an EPLIN-specific cell component and detecting binding of the reagent to the component;
b) contacting a second cell not having a cell proliferative disorder with a reagent bthat binds to an EPLIN-specific cell component and detecting binding of the reagent to the component;
c) comparing the level of binding of the reagent in the first sample with the level of binding of the reagent in the second sample, wherein a decreased level of binding of the reagent to an EPLIN-specific cell component from the first sample is indicative of a cell proliferative disorder.

48. The method of claim 47, wherein the EPLIN-specific cell component is nucleic acid which encodes the EPLIN polypeptide.

49. The method of claim 47, wherein the EPLIN-specific cell component is the EPLIN protein, or fragments thereof.

50. The method of claim 48, wherein the nucleic acid is RNA.

51. The method of claim 47, wherein the reagent is a probe.

52. The method of claim 51, wherein the probe is nucleic acid.

53. The method of claim 51, wherein the probe is an antibody.

54. The method of claim 53, wherein the antibody is a human antibody.

55. The method of claim 53, wherein the antibody is polyclonal.

56. The method of claim 54, wherein the antibody is monoclonal.

57. A kit useful for the detection of an EPLIN-specific cell component, the kit comprising carrier means containing one or more containers comprising a first container containing an EPLIN-specific binding reagent.

58. A method for ameliorating a cell proliferative disorder associated with EPLIN activity, comprising treating a subject having the disorder with a compound that regulates EPLIN activity.

59. The method of claim 58, wherein the cell proliferative disorder is selected from the group consisting of cancer, atherosclerosis, Gaucher disease, scleroderma, arthritis and liver cirrhosis.

60. The method of claim 58, wherein the compound that regulates EPLIN activity is a protagonist of EPLIN.

61. A method for ameliorating a cell proliferative disorder associated with EPLIN, comprising treating a subject having the disorder, at the site of the disorder, with a composition which regulates EPLIN activity.

62. An isolated, synthetic, or recombinant polynucleotide comprising a epithelial lost in neoplasm (EPLIN) non-coding regulatory sequence.

63. The polynucleotide of claim 62, wherein the non-coding regulatory sequence comprises at least 15, at least 50, at least at 100, at least 200, or at least 500 bases as set forth in SEQ ID NO:15 or SEQ ID:NO16.

64. An isolated, synthetic, or recombinant polynucleotide comprising a epithelial lost in neoplasm (EPLIN) non-coding regulatory sequence operably linked to a heterogenous sequence.

65. The polynucleotide of claim 64, wherein the heterogenous sequence encodes a protein.

66. The polynucleotide of claim 65, wherein the protein is a cellular toxin.

67. The polynucleotide of claim 65, wherein the protein is detectable by fluorescence, phosphorescence, or by virtue of its possessing an enzymatic activity.

68. The polynucleotide of claim 67, wherein the detectable protein is selected from the group consisting of firefly luciferase, alpha-glucuronidase, alpha-galactosidase, chloramphenicol acetyl transferase, green fluorescent protein, enhanced green fluorescent protein, and the human secreted alkaline phosphatase.

69. A nucleic acid construct the sequence of which comprises SEQ ID NO:15 operably associated to a heterologous coding sequence.

70. A nucleic acid construct the sequence of which comprises SEQ ID NO:16 operably associated to a heterologous coding sequence.

71. A vector comprising the nucleic acid construct of claims 69 and 70.

72. A nucleic acid construct comprising at least one EPLIN non-coding regulatory sequence the sequence of which comprises SEQ ID NO:15; and a heterologous nucleic acid sequence operatively linked to the regulatory sequence, wherein expression of the heterologous sequence is regulated by the non-coding sequence.

73. A nucleic acid construct comprising at least one EPLIN non-coding regulatory sequence the sequence of which comprises SEQ ID NO:16; and a heterologous nucleic acid sequence operatively linked to the regulatory sequence, wherein expression of the heterologous sequence is regulated by the non-coding sequence.

74. A method for screening for a compound that binds to a EPLIN non-coding regulatory sequence, comprising:
a) providing an isolated, synthetic, or recombinant polynucleotide comprising a EPLIN non-coding regulatory sequence and a test compound,
b) contacting the polynucleotide with the test compound, and
c) measuring the ability of the test compound to bind to the polynucleotide.

75. A method for screening for a compound that modulates EPLIN non-coding regulatory sequence activity, comprising:
a) providing a first polynucleotide comprising an isolated, synthetic, or recombinant EPLIN non-coding regulatory sequence operably linked to a heterogenous sequence, and a test compound,
b) contacting the polynucleotide with the test compound, and
c) measuring the ability of the test compound to modulate transcription of the heterogenous sequence.

76. The method of claim 75, wherein the heterogenous sequence encodes a protein.

77. The method of claim 76, wherein protein is detectable by fluorescence or phosphorescence or by virtue of its possessing an enzymatic activity.

78. The polynucleotide of claim 77, wherein the detectable protein is selected from the group consisting of firefly luciferase, alpha-glucuronidase, alpha-galactosidase, chloramphenicol acetyl transferase, green fluorescent protein, enhanced green fluorescent protein, and the human secreted alkaline phosphatase.

79. A method of expressing a heterologous nucleic acid sequence in a cell comprising:
a) transforming the cell with a polynucleotide polynucleotide of claim 61; and
b) growing the cell under conditions where the heterologous nucleic acid sequence is expressed in the cell.

80. An isolated nucleic acid molecule which hybridizes to SEQ ID NO:15 or SEQ ID NO:16 under stringent hybridization conditions.
